(19) United States
(12) Patent Application Publication
(10) Pub. No.: US 2024/0280590 A1
(43) Pub. Date: Aug. 22, 2024

(54) COMPOSITIONS AND METHODS FOR FACIOSCAPULOHUMERAL MUSCULAR DYSTROPHY BIOMARKER DETECTION

(71) Applicant: University of Washington, Seattle, WA (US)

(72) Inventors: Joel R. Chamberlain, Seattle, WA (US); Jan Christian Lood, Seattle, WA (US)

(73) Assignee: University of Washington, Seattle, WA (US)

(21) Appl. No.: 18/582,346

(22) Filed: Feb. 20, 2024

Related U.S. Application Data
(60) Provisional application No. 63/486,230, filed on Feb. 21, 2023.

(51) Int. Cl. G01N 33/68

(52) U.S. Cl. CPC G01N 33/6896; G01N 2800/2878; G01N 2800/52

(57) ABSTRACT

Methods and compositions for diagnosing or detecting Facioscapulohumeral Dystrophy (FSHD) are provided. One aspect provides a method for the evaluation of risk, progression, and therapeutic response of FSHD in human subjects. The method includes measuring/detecting an overall expression pattern or expression level of neutrophil extracellular traps (NETs) in one or more biological samples obtained from the subject and comparing the measured levels of these NETs to overall expression pattern or level of the neutrophil extracellular traps (NETs) from a biological sample of a normal subject. The method further comprises detecting the expression level of a mitochondrial protein in the one or more biological samples of the subject.

### CROSS-REFERENCES TO RELATED APPLICATIONS

This application claims the benefit of U.S. Provisional Application No. 63/486,230, filed Feb. 21, 2023, of which is incorporated herein by reference in its entirety.

### STATEMENT OF GOVERNMENT LICENSE RIGHTS

This invention was made with government support under Grant Nos. W81XWH-21-1-0006 and W81XWH-21-1-0007, awarded by the Department of Defense and Grant No. R35HL145262, awarded by the National Heart, Lung, and Blood Institute and Grant Nos. K25HL135432 and R01DK117914 and U01DK127553, awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1295USUW_Seq_List_20240214.xml. The XML file is 51,175 bytes; was created on Feb. 14, 2024; and is being submitted electronically via Patent Center with the filing of the specification.

### BACKGROUND

Facioscapulohumeral dystrophy (FSHD) is an adult, slow progressing muscle wasting disease caused by a dominant mutation in DUX4, a primarily developmental gene, and is the third most common muscular dystrophy. The most prevalent form of FSHD (FSHD1 or Type 1A) is caused by the deletion of a subset of D4Z4 macrosatellite repeats in the subtelomeric region of chromosome 4q. Unaffected individuals have 11-100 of the 3.3 kb D4Z4 repeat units, whereas FSHD individuals have 10 or fewer repeats. The DNA mutation results in aberrant expression of DUX4 in adult muscle. FSHD can be genetically diagnosed by the contraction of the number of D4Z4 repeats to 10 or fewer repeats on a specific 4qA haplotype. Currently, the diagnostic test for FSHD requires genetic testing involving pulse-field gel electrophoresis and Southern blotting to detect the contraction of the D4Z4 repeats. These prior methods are time consuming, labor intensive, not precise, and very expensive. Thus, there remains a great need for rapid, economical, sensitive, and accurate detection assays to detect and characterize the status of FSHD, including prediction of the disease activity or flares, to support precise medical intervention. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Surprisingly, and as a byproduct of investigating disease caused by DUX4 expression, inventors found that neutrophil extracellular traps (NETs)-associated proteins were detected at higher levels in Facioscapulohumeral Dystrophy (FSHD) blood plasma compared to control subjects not suffering from FSHD, including neutrophil elastase bound-DNA (NE-DNA), myeloperoxidase bound-DNA (MPO-DNA), and S100A8/S100A9 (calprotectin). These findings are unexpected as FSHD is not considered an inflammatory myopathy. In addition to the neutrophil derived NETs-associated proteins inventors also assayed for several muscle mitochondrial related proteins (based on data from mouse studies) and found that growth and differentiation factor 15 (GDF-15), a muscle cytokine, was upregulated in FSHD patient plasma.

Accordingly, in one aspect, the present disclosure provides a method for diagnosing or detecting Facioscapulohumeral Dystrophy (FSHD) in a subject. In some embodiments, the method comprises: (i) obtaining one or more biological samples from the subject; (ii) measuring/detecting an overall expression pattern or expression level of at least one neutrophil extracellular traps (NETs)-associated protein in the one or more biological samples obtained from the subject; and (iii) comparing the overall expression pattern or expression level of the at least one NETs-associated protein from the one or more biological samples of the subject with the overall expression pattern or level of the at least one NETs-associated protein in a reference biological sample. In some embodiments, detecting an expression or overexpression of the at least one NETs-associated protein, relative to the reference biological sample is indicative of FSHD. In some embodiments, the method further comprises detecting the expression level of at least one mitochondrial protein and comparing it to an expression level of the at least one mitochondrial protein in the reference sample. In some embodiments, detecting an overexpression of the at least one mitochondrial protein relative to the expression of the at least one mitochondrial protein in the reference sample is indicative of FSHD.

In some embodiments, the reference sample is a biological sample obtained from a healthy subject, wherein the healthy subject is a subject not suffering from or at risk for FSHD. In some embodiments, the reference sample is a biological sample corresponding to the biological sample obtained from the subject. In some embodiments, the one or biological sample and/or the reference sample is selected from blood, serum, plasma, synovial fluid, bronchoalveolar lavage, spinal fluid, and saliva.

In some embodiments, detecting an overall expression pattern or expression level of the at least one NETs-associated protein comprises detecting the overall expression pattern or expression level of at least one of Neutrophil Elastase-DNA (NE-DNA), Myeloperoxidase-DNA (MPO-DNA), and calprotectin.

In some embodiments, the at least one mitochondrial protein is selected from at least one of N-formyl methionyl peptides (fMET), growth and differentiation factor 15 (GDF-15), 8-hydroxy-2′-deoxyguanosine (8-OHdG), and MT-ND6 protein.

In another aspect, the present disclosure provides a method of diagnosing and/or detecting FSHD in a subject. In some embodiments, the disclosure provides a method for monitoring/detecting the expression of one or more NETs-associated protein and at least one mitochondrial protein, disclosed herein. In some embodiments, the method further comprises monitoring/detecting the expression of at least one cytokine. In some embodiments, the cytokine is IL-6. In some embodiments, the method is useful for predicting patients who will progress (prognostic biomarker), and/or predict patients that will respond to drug (predictive biomarker).

In some embodiments, the method comprises (i) obtaining one or more biological samples from the subject; (ii) measuring/detecting an overall expression pattern or expression level of: (a) at least one NETs-associated protein, and (b) at least one mitochondrial protein, in the one or more biological samples obtained from the subject; and (iii) comparing the overall expression pattern or expression level of: (a) the at least one NETs-associated protein, and (b) the at least one mitochondrial protein, from the one or more biological samples of the subject, with the overall expression pattern or level of the at least one NETs-associated protein and at least one mitochondrial protein in a reference sample. In some embodiments, detecting an expression or overexpression of the at least one NETs-associated protein and the at least one mitochondrial protein, relative to the reference sample identifies the subject as having FSHD. In some embodiments, detecting an expression or overexpression of the at least one NETs-associated protein and the at least one mitochondrial protein, relative to the reference sample associates with changes in demographic and/or clinical outcome or functional changes over time.

In some embodiments, the reference sample is a biological sample obtained from a healthy subject, wherein the healthy subject is a subject not suffering from or at risk for FSHD. In some embodiments, the reference sample is a biological sample corresponding to the biological sample obtained from the subject. In some embodiments, the one or more biological sample and/or the reference sample is selected from blood, serum, plasma, synovial fluid, bronchoalveolar lavage, spinal fluid, and saliva.

In some embodiments, the at least one NETs-associated protein is selected from Neutrophil Elastase-DNA (NE-DNA), Myeloperoxidase-DNA (MPO-DNA), and calprotectin.

In some embodiments, the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

In another aspect, the present disclosure provides a method of treating a subject identified as suffering from, or at risk for developing FSHD. In some embodiments, the method comprises administering to the subject an effective amount of at least one therapeutic agent. In some embodiments, the subject is determined to be suffering from, or at risk for developing FSHD by a method comprising: (i) obtaining one or more biological samples from the subject suffering from, or at risk for developing FSHD; (ii) measuring/detecting an overall expression pattern or expression level of at least one NETs-associated protein in the one or more biological samples obtained from the subject suffering from, or at risk for developing FSHD; and (iii) comparing the overall expression pattern or expression level of the at least one NETs-associated protein from the one or more biological samples of the subject suffering from, or at risk for developing FSHD, with the overall expression pattern or level of the at least one NETs-associated protein from a reference sample. In some embodiments, detecting an expression or overexpression of the at least one neutrophil extracellular traps (NETs)-associated protein, relative to the reference sample identifies the subject as suffering from, or at risk for developing FSHD.

In some embodiments, the method further comprises detecting an expression level of at least one mitochondrial protein and comparing it to an expression level of the at least one mitochondrial protein in the reference sample. In some embodiments, detecting an overexpression of the at least one mitochondrial protein relative to the expression of the at least one mitochondrial protein in the reference sample is indicative of FSHD.

In some embodiments, the reference sample is a biological sample obtained from a healthy subject, wherein the healthy subject is a subject not suffering from or at risk for FSHD. In some embodiments, the reference sample is a biological sample corresponding to the biological sample obtained from the subject. In some embodiments, the one or biological sample and/or the reference sample is selected from blood, serum, plasma, synovial fluid, bronchoalveolar lavage, spinal fluid, and saliva.

In some embodiments, the step of administering an effective amount of at least one therapeutic agent comprises administering an effective amount of at least one of DUX4 inhibitor, an anti-inflammatory agent, or a GDF-15 inhibitor, or a combination thereof.

In another aspect, the present disclosure provided a kit, comprising: a first group of capture affinity reagents that bind to at least one NETs-associated protein at a first epitope, and a first group of detection affinity reagents that bind to the at least one NETs-associated protein at a second epitope. In some embodiments, the first group of capture affinity reagents are specific for and bind to the at least one NETs-associated protein at the first epitope. In some embodiments, the first group of detection affinity reagents are specific for and bind to double-stranded DNA. In some embodiments, the at least one NETs-associated protein is selected from NE-DNA, MPO-DNA, and calprotectin.

In some embodiments, the kit further comprises a second group of capture affinity reagents that bind to a first epitope of at least one mitochondrial protein; and a second group of detection affinity reagents that bind to an epitope of the second group of capture affinity reagents. In some embodiments, the second group of capture affinity reagents are specific for and bind to the at least one mitochondrial protein at the first epitope. In some embodiments, the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

In some embodiments, the first group of capture affinity reagents are immobilized on a solid substrate. In some embodiments, the second group of capture affinity reagents are immobilized on a solid substrate.

In some embodiments, the method further comprises contacting the biological sample with a third group of detection affinity reagents that specifically bind to the first group of detection affinity reagents and/or the first group of capture affinity reagents. In such embodiments, the third group of detection reagents have detectable labels and serve to provide a detectable signal on the at least one bound and immobilized NETs-associated protein.

In some embodiment, the method further comprises contacting the biological sample with a fourth group of detection affinity reagents that specifically binds to the second group of detection affinity reagent. In such embodiments, the fourth group of detection reagents have detectable labels and serve to provide a detectable signal on the at least one bound and immobilized mitochondrial protein.

The present disclosure thus provides methods, reagents, and kits for assessing the risk or presence of FSHD in a subject, and further provides methods for treating a subject suffering from or at risk for developing FSHD.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIG. 1 depicts the clinical features of Facioscapulohumeral Dystrophy (FSHD);

FIG. 2 is a schematic depicting FSHD genetic mutation and resulting molecular changes leading to DUX4 expression. Chromosome telomeric repeat region D4Z4 at 4q35 is shortened from 11-100 of the 3.3 Kb microsatellite repeats to <10. Loss of repeats results in reduction in DNA CpG methylation and chromatin modifications that result in a transition to more relaxed chromatin and an increase in transcription of the repeat-embedded DUX4 gene. Stable polyadenylated DUX4 mRNA is produced from the pathogenic allele containing a functional polyadenylation sequence;

FIG. 3 is a schematic showing FSHD changes in genetic and epigenetic regulation of the D4Z4 region and resulting cellular changes resulting from DUX4 expression. Several factors affect genetic and epigenetic regulation of DUX4 (left). DUX4 is normally expressed in testis and thymus, but also can be detected in myoblasts grown in culture dishes and lymphoblastoid cells in blood from FSHD patients (center). Expression of DUX4 leads to numerous cellular changes in cellular metabolism highlighted (right);

FIG. 4 shows the Adeno-associated virus (AAV) vector delivery of the endogenous DUX4 gene and promoter region from the D474 final repeat region (SEQ ID NO: 1) to wild-type (WT) mouse muscle. Recombinant AAV vector used for gene delivery was engineered to express the last repeat from the human pathogenic allele of clone λ42 followed by a strong polyadenylation sequence. Vectors were made with AAV serotype 6 (AAV6) capsids for local or systemic targeted delivery of DUX4 to muscle of WT mice to create the AAV-DUX4 mouse;

FIG. 5 shows rAAV-DUX4 vector (SEQ ID NO: 1) delivery of the endogenous DUX4 gene to WT mouse tibialis anterior (TA) muscle recapitulates structural changes seen in FSHD muscle. Hematoxylin and eosin stained transverse cryosections highlight focal changes with low dose (5×10⁹ vector genomes [vg]) versus more widespread effects with high dose (2×10¹⁰ vg);

FIG. 6 shows rAAV-DUX4 vector (SEQ ID NO: 1) delivery of the endogenous DUX4 gene to WT mouse tibialis anterior muscle recapitulates ultrastructural changes in FSHD muscle. Gluteraldehyde and paraformaldehyde fixed and osmium tetroxide-stained electron microscopic sections highlight muscle ultrastructural changes with high and low dose vector delivery (top panels; scale=5 μm left, 1 μm right) compared to control muscle (lower panel; scale=1 μm), including nuclear shape irregularities and mitochondrial cristae deformation (white arrows) and loss (yellow arrows);

FIG. 7 shows Principal Component Analysis (PCA) of mRNA profiling of 5 samples each of high dose rAAV-DUX4 (2×10¹⁰ vgs) or empty capsid (viral shell, no DNA, same protein amount) injected TA muscles harvested at 2 weeks post injection. RNA was isolated from rAAV-DUX4 (SEQ ID NO: 1) or empty capsid injected muscle for RNA sequencing (RNAseq). PCA comparison of the 5 samples in each group show a clear grouping of individual samples with empty capsid (control) samples clustering at the top left and rAAV-DUX4 samples (treatment) at the bottom right;

FIGS. SA-8B. show RNAseq analysis reveals top downregulated cellular pathways with rAAV-DUX4 versus empty capsid injected TA muscles. Qiagen Ingenuity Pathway Analysis reveals downregulation of genes for skeletal muscle development (FIG. 8A) and function and lipid metabolism (FIG. 8B). FSHD muscle is weakened in disease and published data from other groups highlights the changes in lipid metabolism and the increase in presumptive fibroadipogenic progenitors (FAPs):

FIGS. 9A-9B. show RNAseq analysis of rAAV-DUX4 injected mouse TA muscle highlighting differential regulation of cell cycle and immune response genes as top annotation clusters for Gene Ontology (GO) Term Biological Process. Data collected from mRNA profiling of FSHD-derived muscle previously showed differences in expression of cell cycle genes and immune gene altered responses (FIG. 9A). The rAAV-DUX4 injected muscle also shows this response and highlights the top immune related change in ‘NEUTROPHIL CHEMOTAXIS’ with a false discovery rate (FDR) of 1.12×10⁻⁰⁸ with inflammatory response, chemokine signaling (interleukin 1 beta [IL-1B] and interferon gamma [IFNG]), and chemotaxis of monocytes and lymphocytes (FIG. 9B). These data implicate neutrophils in early immune response to DUX4 expression;

FIG. 10 shows innate immune genes upregulated in rAAV-DUX4 injected mouse muscle. Volcano Plot highlighting the genes involved in the innate immunity response with a subset of these genes highlighted. Many of these upregulated genes are involved in an IFN-mediated response to DUX4 expression;

FIGS. 11A-11B show results from an initial Pilot study showing detection of dsDNA-bound MPO (MPO-DNA) resulted in a significant difference in the means for MPO-DNA (p=0.0193 MPO). Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 12A-12B show results from an Extended Pilot study with a total of 11 FSHD samples and 13 controls, showing a significant difference in the mean for MPO-DNA (p=0.0006 MPO). Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 13A-13B show results from an Extended Pilot study with a total of 11 FSHD samples and 13 controls, showing a significant difference in the mean for NE-DNA (p=0.0002 NE). Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 14A-14B show results from an Extended Pilot study with a total of 11 FSHD samples and 13 controls, showing a significant difference in the mean for Calprotectin (S100A8/S100A9) (p=0.0038 calprotectin). Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 15A-15C show results for NE-DNA (FIG. 15A) MPO-DNA (FIG. 15B), and Calprotectin (FIG. 15C) as biomarkers for FSHD, using patient tissue samples obtained from the Wellstone Study collection. A high significance for MPO-DNA and NE-DNA (p<0.0001) and lower significance for calprotectin (p=0.0083) was seen. Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 16A-16C plot the difference between means. The greatest difference in the mean in the Student's t test was again for NE-DNA with double the concentration difference (p=0.0001) (FIG. 16A). The controls did not show much variation compared to the FSHD samples which are expected to vary widely due to the wide-ranging extent of involvement of different muscles in the disease. Note that overall concentration values are different from the earlier studies and reflect the concentration relative to a different standard curve values used in this study. The differences in the means for MPO-DNA (FIG. 16B) and calprotectin (FIG. 16C) were significant, p=0.0001 and p=0.0083, respectively, although samples from both groups had overlapping values. Statistics were carried out using Prism (GraphPad) with the Student's t-test (unpaired, 2-tailed, parametric, assuming Gaussian distribution, with Welch's correction [no assumption of same SD], standard error of the mean, (SEM);

FIGS. 17A-17C show the relationship of age-adjusted clinical severity score (ACSS) for patients compared to the level of NE-DNA (FIG. 17A), MPO-DNA (FIG. 17B), and calprotectin (FIG. 17C). Using calculations of Pearson's correlation coefficient, a weak correlation between ACSS for both MPO-DNA (r=0.3490; p=0.0271) (FIG. 17B) and calprotectin (r=0.3767; p=0.0183) (FIG. 17C) was observed;

FIG. 18 shows the spatial genomic data from aggregated DUX4 expression supporting multiplexing Neutrophil Biomarkers with Mitochondrial Biomarkers for Detection in patient sera. Uniform Manifold Approximation and Projection (UMAP) dimensionality reduction is shown from spatial genomics performed on AAV-DUX4 injected low and high dose cryopreserved transverse TA muscle sections (n=4; arrows highlight DUX4 expression clusters of aggregated data compared to a control section compared to bulk [no DUX4 expression]). Differential gene expression analysis of areas with DUX4 expression is depicted as colored spots in the Seurat clusters. Analysis of differentially expressed gene-defined pathways in the Seurat clusters associated with high and low frequency of DUX4 expression include suppression pathways in both high and low clusters, in addition to muscle function pathways in regions of high DUX4 expression;

FIG. 19 shows spatial genomic data from nearest neighbor expression moving away from DUX4 expression, which complements unlayered/ungrouped Seurat cluster analysis (FIG. 18) to support multiplexing neutrophil biomarkers with mitochondrial biomarkers for detection in patient sera (each spot of 50 μm is an individual expression library). Expression profiles from individual RNAseq libraries are mapped onto the 4 muscle transverse sections in layers relative to regions of DUX4 expression. First layer away from DUX4 (nearest neighbor 1, nb1) with seurat clustering of neighbor layers relative to bulk (no DUX4 expression) is highlighted with arrows indicating clusters of high and low DUX4 expression. A single section (also shown in the 4-panel map) is displayed from low dose injection and indicates a single focus of DUX4 expression with nearest neighbor spots corresponding to unique expression libraries. Pathway analysis of nb layers from the aggregated data shows suppression of mitochondrial and muscle function pathways at DUX4 expression and activation of immune system gene expression at DUX4 nb1, with arrow highlighting ‘regulation of leukocyte activation.’ Leukocytes are defined as white blood cells that include neutrophils among others and implicate an immediate mitochondria and immune cell response spatially related to DUX4 expression;

FIG. 20 shows spatial genomic data for a TA muscle sections from single low dose AAV-DUX4 treatment analyzed for Wfdc3 gene expression (a DUX4 downstream target gene related to the innate immune response) expression supporting multiplexing Neutrophil biomarkers with Mitochondrial biomarkers for detection in patient plasma. Wfdc3 is more widely expressed as shown at the region of active DUX4 expression in FIG. 19, as well as areas further away, either acting as marker of previous pulsatile DUX4 expression or as a spatial signaling factor. Pathway analysis of Wfdc3 and nb1 of Wfdc3 indicates overlapping changes in mitochondrial and muscle function pathway suppression with immune cell activation. Activation of angiogenic pathways in the Wfdc3 nb1 layer is also observed, which has been associated with DUX4 expression in mouse muscle implicating a factor like VEGF could be a multiplexed biomarker with immune and mitochondrial agents;

FIGS. 21A-21B show GDF-15 expression levels are increased in the FSHD plasma in a pilot study of 11 FSHD patient and 11 control plasma samples (215 pg/ml control vs. 530 pg/ml FSHD; p<0.0001 Student's t test). GDF-15 is a mitochondrial elicited myokine and an example of mitochondrial derived agents for FSHD biomarker multiplexing. Statistics were carried out using Prism (GraphPad) with the Student's t-test (unpaired, 2-tailed, parametric, assuming Gaussian distribution, with Welch's correction [no assumption of same SD], standard error of the mean, (SEM); and

FIGS. 22A-22B show a GDF-15 expression level increase in the FSHD plasma in an Exploratory Study of Wellstone Study participants (p<0.0054 Student's t test). Statistics were carried out using Prism (GraphPad) with the Student's t-test (unpaired, 2-tailed, parametric, assuming Gaussian distribution, with Welch's correction [no assumption of same SD], standard error of the mean, (SEM);

FIG. 23 shows exemplary multiplex Biomarker assays for detection of components of NETs and/or mitochondrial proteins, including a Meso Scale Discovery (MSD) sandwich assay, a sandwich ELISA with Colorimetric detection, and the Luminex® Assay that can be used to perform the methods of the present disclosure;

FIG. 24 shows method of detecting neutrophil extracellular traps (NETs). A sandwich Enzyme-Linked ImmunoSorbent Assay (ELISA) used for distinguishing DNA-bound enzymes produced from neutrophil activation vs. enzymes not associated with DNA. Plates are coated with antibodies with specificity for the enzymes, here either neutrophil elastase (NE; anti-NE Abs) or myeloperoxidase (MPO; anti-MPO Abs). After washing to remove non-specific interactions a double-stranded DNA antibody (dsDNA Ab) is added that attaches to the DNA bound to the enzymes. The anti-dsDNA Ab is detected by adding a light substrate that is activated by the anti-dsDNA Ab-conjugated horseradish peroxidase enzyme to produce quantifiable light emission; and

FIGS. 25A-25D show ELISA detection of NE-DNA, MPO-DNA, Calprotectin in blood plasma samples obtained from large patient cohorts:t (cohort 1; ReSolve Study) (FIG. 25A) and from patient cohort 2 (Wellstone Study) (FIG. 25B), compared to adult control samples and compared to Duchenne muscular dystrophy (DMD) samples with age-matched child controls as a distinct population of muscular dystrophy patients. Each drawing shows 3 panels of 2 sample populations separately (one-way ANOVA), pooled populations for cohort 1 (Student's t test; Mann-Whitney), and a Receiver Operator Curve (ROC) with the area under the curve (AUC) indicated (cohort 2 not pooled because 2nd population is from repeat blood draw). Also shown are ELISA results of GDF-15 concentrations in plasma for patient cohorts 1 and 2 (FIG. 25C). Multivariate correlation analyses of the NET-DNA and GDF-15 ELISA results with FSHD demographic and clinical functional outcome measures show a relationship of GDF-15 levels with several clinical measures and age (FIG. 25D) (Thickness of circle line defines significance according to p value, ½ point line p<0.05, 1-point line p<0.001, 2-point line p<0.005, and 3-point line p<0.001). All comparisons of NET-DNA concentrations from FSHD samples vs. controls indicate significant difference in the means of these complexes in blood obtained from large patient cohorts in pooled samples (Student's t test; Mann-Whitney U comparison), although individual multiple comparisons of some populations within cohorts showed less significance in the mean differences with one-way ANOVA analysis [GraphPad]).

### DETAILED DESCRIPTION

The present disclosure provides compositions and improved methods for detection, characterization, and monitoring of Facioscapulohumeral Dystrophy (FSHD). The disclosed methods and compositions can be incorporated into treatment strategies to address such conditions in a more accurate and precise approach.

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

As used herein, the term “healthy human subject” or “healthy subject” or “healthy control subjects” refers to an individual/subject who is known not to suffer from FSHD, such knowledge being derived from clinical data on the individual/subject.

As used herein, the term “biological sample” refers to any type of material of biological origin isolated from a subject, including, for example, DNA, RNA, protein, such as, for example, blood, plasma, serum, fecal matter, urine, semen, bone marrow, bile, spinal fluid, tears, saliva, muscle biopsy, organ tissue or other material of biological origin known by those of ordinary skill in the art.

Biological samples also include blood, serum, plasma, synovial fluid, bronchoalveolar lavage, spinal fluid, saliva, and the like. In some embodiments, the biological sample from the subject comprises blood, serum, plasma, synovial fluid, bronchoalveolar lavage, spinal fluid, saliva, and the like including any bodily fluid.

As used herein, “a subject” includes all mammals, including without limitation, humans, non-human primates, dogs, cats, horses, sheep, goats, cows, rabbits, pigs, and rodents.

As used herein, the term “therapeutically effective amount” is an amount of an agent of the invention that alleviates, totally or partially, the pathophysiological effects of FSHD. The amount will depend on, for example, the subject size, gender, magnitude of the associated condition or injury, and the like. For a given subject in need thereof, a therapeutically effective amount can be determined by those of ordinary skill in the art by methods known to those of ordinary skill in the art.

For convenience, certain terms employed in this description and/or the claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively.

The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “epitope” refers to a group of amino acids, e.g., antigenic determinant, or other chemical groups exposed on the surface of a molecule.

The term “affinity reagent” refers to any molecule having an ability to bind to a specific target molecule (i.e., antigen of interest and/or target antigen and/or a specific chemical group) with a specific affinity (i.e., detectable over background). Affinity reagent molecules are known and have been characterized for useful antigens and are encompassed by the present application without limitation. Exemplary and non-limiting categories of affinity reagents that can be used in the context of the present disclosure include antibodies, and antigen fragments and derivatives thereof.

The term “antibody” is used herein in the broadest sense and encompasses various antibody structures derived from any antibody-producing mammal (e.g., mouse, rat, rabbit, and primate including human), and which specifically bind to an antigen of interest. An antibody fragment specifically refers to an intact portion or subdomain of a source antibody that still retains antigen-biding capability. An antibody derivative refers to a molecule that incorporates one or more antibodies or antibody fragments. Typically, there is at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure include multispecific antibodies (e.g., bispecific antibodies); humanized antibodies; murine antibodies; chimeric, mouse-human, mouse-primate, primate-human monoclonal antibodies; and anti-idiotype antibodies.

As indicated, an antibody fragment is a portion or subdomain derived from or related to a full-length antibody, preferably including the complementarity-determining regions (CDRs), antigen binding regions, or variable regions thereof, and antibody derivatives refer to further structural modification or combinations in the resulting molecule. Illustrative examples of antibody fragments or derivatives encompassed by the present disclosure include Fab, Fab′, F(ab)2, F(ab′)2 and Fv fragments, diabodies, single-chain antibody molecules, VHH fragments, VNAR fragments, multispecific antibodies formed from antibody fragments, nanobodies and the like. For example, an exemplary single chain antibody derivative encompassed by the disclosure is a “single-chain Fv” or “scFv” antibody fragment, which comprises the VH and VL domains of an antibody, wherein these domains are present in a single polypeptide chain. The Fv polypeptide can further comprise a polypeptide linker between the VH and VL domains, which enables the scFv to form the desired structure for antigen binding. Another exemplary single-chain antibody encompassed by the disclosure is a single-chain Fab fragment (scFab).

As indicated, antibodies can be further modified to created derivatives that suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin. Any of these antibodies, or fragments or derivatives thereof, are encompassed by the disclosure.

Antibody fragments and derivatives that recognize specific epitopes can be generated by any technique known to those of skill in the art. For example, Fab and F(ab′)2 fragments of the disclosure can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain. Further, the antibodies, or fragments or derivatives thereof, of the present disclosure can also be generated using various phage display methods known in the art. Finally, the antibodies, or fragments or derivatives thereof, can be produced recombinantly according to known techniques.

It will be apparent to the skilled practitioner that the affinity reagents can comprise binding domains other than antibody-based domains, such as peptidobodies, antigen-binding scaffolds (e.g., DARPins, HEAT repeat proteins, ARM repeat proteins, tetratricopeptide repeat proteins, and other scaffolds based on naturally occurring repeat proteins, etc. [see, e.g., Boersma and Pluckthun, Curr. Opin. Biotechnol. 22:849-857, 2011, and references cited therein, incorporated herein by reference]), which include a functional binding domain or antigen-binding fragment thereof.

As used herein, the term “treat” refers to medical management of a disease, disorder, or condition (e.g., FSHD) of a subject (e.g., a human or non-human mammal, such as another primate, horse, dog, mouse, rat, guinea pig, rabbit, and the like). Treatment can encompass any indicia of success in the treatment or amelioration of a disease or condition, including any parameter such as abatement, remission, diminishing of symptoms or making the disease or condition more tolerable to the subject, slowing in the rate of degeneration or decline, or making the degeneration less debilitating. Specifically in the context of inflammation, the term treat can encompass reducing inflammation, reducing pain associated with inflammation, or reducing the likelihood of recurrence, compared to not having the treatment. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition. The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In the broadest sense, the naturally occurring amino acids can be divided into groups based upon the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His. This grouping of amino acids can be further subclassed as follows. By “uncharged hydrophilic” amino acid is meant either Ser, Thr, Asn or Gln. By “acidic” amino acid is meant either Glu or Asp. By “basic” amino acid is meant either Lys, Arg or His.

As used herein the term “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid.

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The present disclosure provides methods for detection, characterization, and monitoring of Facioscapulohumeral muscular dystrophy (FSHD). The disclosed methods and compositions can be incorporated into treatment strategies to address FSHD diagnosis, treatment, and prognosis allowing for a more accurate and precise approach for FSHD. The inventors have discovered presence of byproducts of cell activation in human blood that has led to identification of FSHD candidate biomarkers that can be collected with a simple blood draw, followed by a standard processing step, then quantitated in an assay adapted for detection of the disease-associated antigens/biomarkers.

Thus, the methods and compositions of the present disclosure advantageously provide an alternative, non-invasive, rapid, and precise, method of detecting/diagnosing FSHD in a subject, predicting the risk of a subject for developing FSHD, predicting whether a subject will respond to a particular therapy, or monitoring a subject's response to treatment, without the need for the cumbersome, time-consuming, labor intensive, and expensive methods for diagnosing FSHD, which currently require genetic testing requiring cumbersome methodologies including pulse-field gel electrophoresis and Southern blotting to detect the contraction of the D4Z4 repeats in DUX4 gene. Further, the methods disclosed herein are also useful for monitoring FSHD. Currently, the only methods to monitor FSHD are by MRI and functional muscle assessments that can be time consuming, expensive, and unreliable in some respects due to extreme variability of disease clinical manifestation; and where there are no reliable blood biomarkers for tracking disease.

The methods disclosed herein are easily repeated and may provide indications of ongoing disease state, and guide treatment of FSHD.

FSHD is not considered an immune muscle disease or myopathy, such as polymyositis or dermatomyositis, where the immune system causes muscle weakness and damage with high levels of inflammatory cell infiltrates. In some individuals FSHD muscle tissue shows infiltrating immune cells, estimated at ˜ 30% of individuals upon muscle biopsy, as often observed in other muscular dystrophies that accompanies muscle cell damage and repair due to monogenic gene mutation. The number of inflammatory cells in FSHD muscle is not at the levels of cell numbers observed in muscle affected by the inflammatory myopathies. In FSHD muscle resident immune cells, such as macrophages, are required for focal repair of muscle, but are secondary to DUX4 expression and damage.

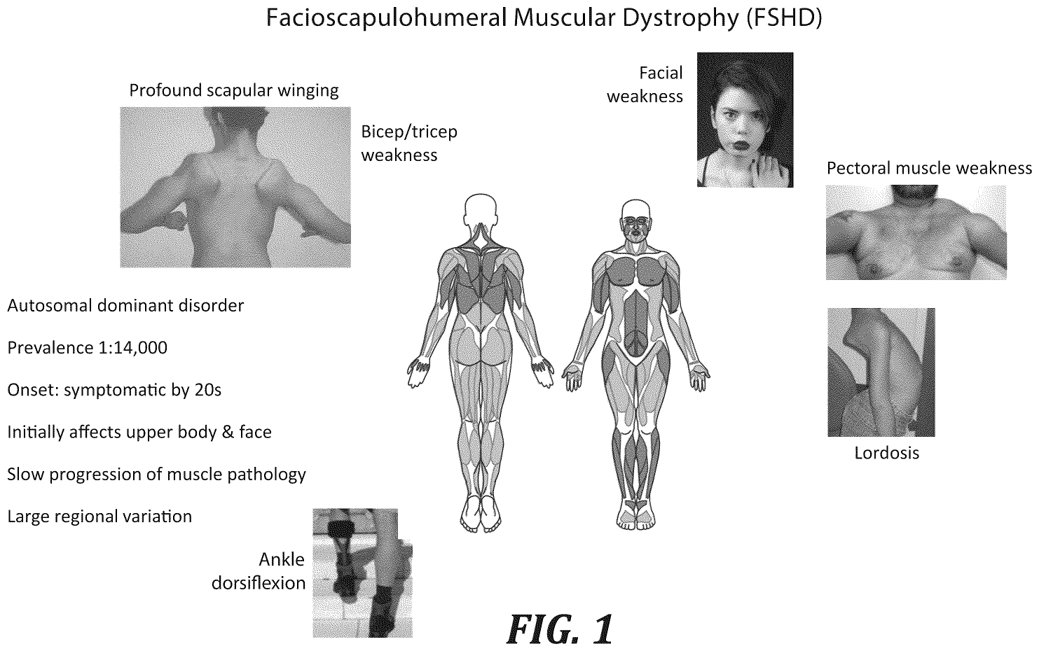

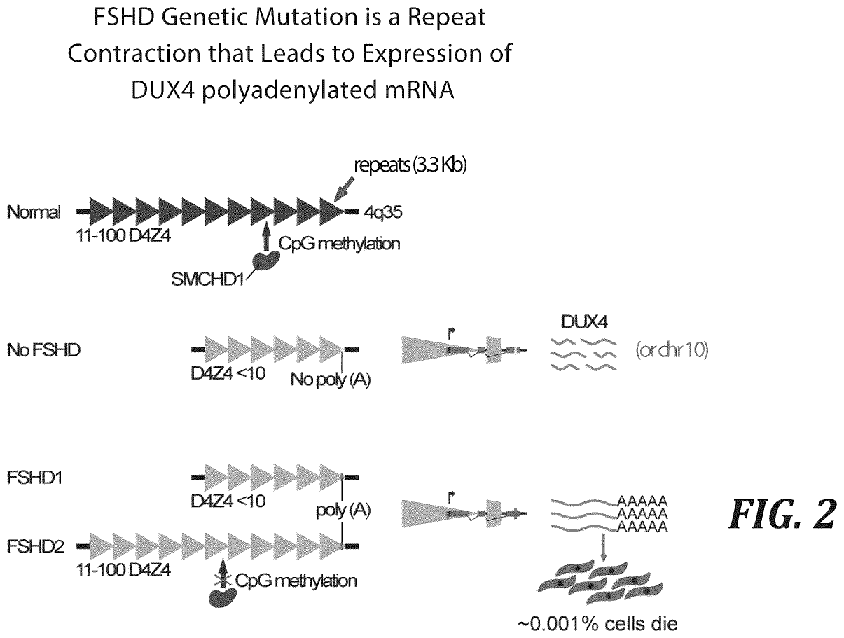

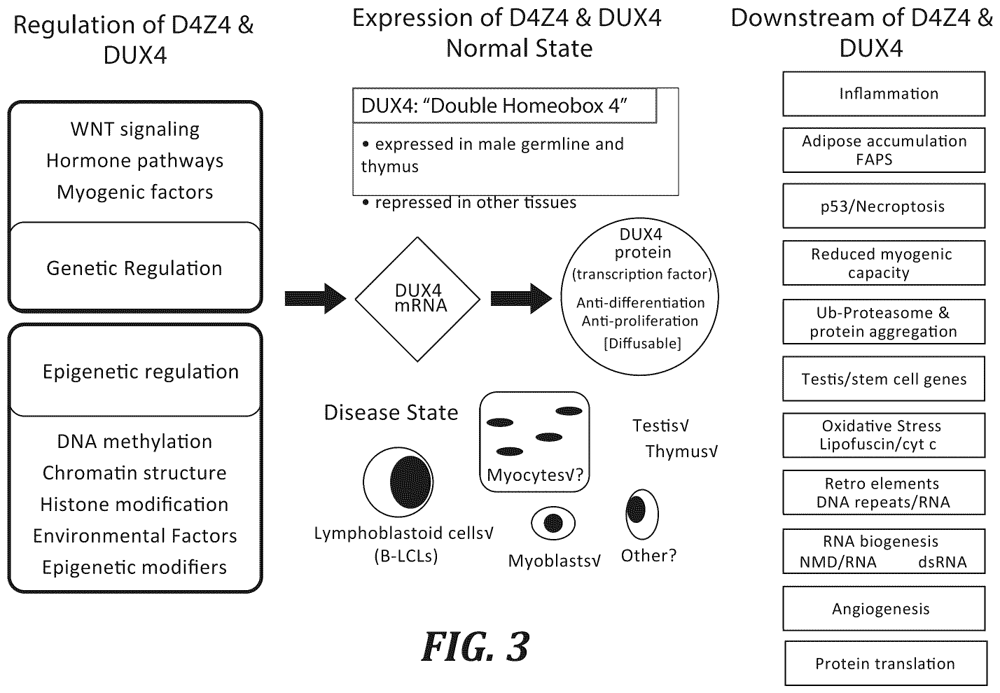

Specifically, FSHD is characterized by an overexpression of DUX4 (FIG. 1) protein, where the overexpression is mediated by a DNA mutation in which a 3.3 kb DNA repetitive region D4Z4 is reduced in repeat numbers (FIG. 2). DUX4 overexpression causes an elevation in oxidative stress, leading to oxidative damage, expression of antioxidative enzymes, induction of DNA damage response, production of reactive oxygen species (ROS), and upregulation of p38/MAPK14, hyaluronidase, HIF1α and other biochemical and metabolic changes in adult muscle (FIG. 3). The expression of DUX4 in FSHD muscle causes focal death of myofibers that becomes more widespread with advancing disease.

Several FSHD studies have shown reactive oxygen species (ROS)-associated mitochondrial involvement in DUX4-mediated cell death, which suggest potential involvement of a leukocyte known to produce ROS, the neutrophil (also known as a polymorphonuclear leukocyte, or PMN. Host immune cells, like PMNs, release large amounts of ROS at the site of infection when their surface receptors are activated. G-protein-coupled receptors (GPCRs), toll-like receptors, and cytokine receptors that encounter ligands can prime PMNs for a greater response. Direct induction of higher levels of ROS production occurs through activation of Fc gamma receptors and integrins. Neutrophil receptor binding activates intracellular signaling pathways, followed by downstream effector protein activation, NADPH oxidase complex assembly, and the concomitant production of ROS by this complex. ROS released by the NADPH oxidase complex can activate intracellular granular protease release, resulting in PMN DNA extrusion and the formation of neutrophil extracellular traps (NETs)/NETs-associated proteins. NETs are composed of DNA-histone complexes with PMN enzymes attached that can cause damage to immobilized pathogens as a mechanism of cell defense.

NETs are produced by the most prevalent of all white blood cells, the neutrophil that is 50-70% of all leukocytes in the circulation. Neutrophils, or polymorphonuclear leukocytes (PMNs), have enzymes packaged inside them for release in response to infections or allergens. They are short lived cells, circulating in the blood stream for no more than 72 hours. When the body encounters an invading microbe, neutrophils migrate from the blood stream and congregate in the infected tissues. PMNs are activated as a primary innate immune effector to perform several functions, including degranulation, cytokine production, and phagocytosis. In addition to germicidal enzyme release with degranulation and signaling to other cells, host immune cells, like PMNs, produce large amounts of reactive oxygen species (ROS) from both NADPH oxidase and mitochondria at the site of infection when their surface receptors, such as integrins and Fc gamma receptors are activated. G-protein-coupled receptors (GPCRs), toll-like receptors, and cytokine receptors that encounter ligands can prime PMNs for a greater response.

The production of Neutrophil Extracellular Traps (NETs) is an exaggerated response to foreign entities, such as bacteria or viruses and by sterile stimuli, such as autoantibodies, cytokines and immune complexes. The term NETs and NETs-associated protein(s) is used interchangeably. NETs are essentially an extrusion of intracellular material composed of DNA-histone complexes with enzymes, such as myeloperoxidase (MPO) and neutrophil elastase (NE), attached that can cause damage to immobilized pathogens as a mechanism of cell defense. NETs formation requires histone citrullination and ROS production. The formation of NETs has been observed to lead to cell death and subsequently referred to as NETosis. There are 2 types of NETosis: suicidal NETosis, a type of programmed cell death, and vital NETosis, in which NET-producing cells remain intact following extrusion and retain some of their effector functions. In some cases, mitochondrial DNA is released from neutrophils that caused induction of a type 1 interferon response as a reaction to oligodeoxynucleotides. The phenomenon of DNA/histone/enzyme extrusion presents an opportunity to detect DNA-associated proteins (nuclear [or mitochondrial-minor presence]) as an indication of the potential presence of neutrophils or other immune cells in this activated state of the neutrophil and potentially other NET-forming immune cells.

For the inflammatory myopathies the immune system is the primary insult, manifesting muscle disease as chronic inflammation and weakness. As stated above the primary muscle insult in FSHD is DUX4 expression, such that FSHD is not considered an inflammatory myopathy. Surprisingly, using a mouse model of FSHD, the inventors have identified neutrophil/PMN involvement in FSHD, and further using an ELISA assay have demonstrated NETs as potential FSHD biomarker in biological samples obtained from FSHD patients.

Because the production of NETs/NETs-associated proteins has been observed and detected in blood samples of immune and autoimmune disease, identification of more than one type of biomarker distinguished by source and activity could, when combined in an ELISA assay, provide a unique concentration signature to distinguish FSHD plasma samples from other diseases. Growth and Differentiation Factor-15 (GDF-15) levels are increased in response to disease states associated with hypoxia, oxidative stress, acute injury and inflammation, and sporadic inclusion body myositis, making it a good candidate as a FSHD biomarker. GDF-15 is a mitochondrial protein and a cytokine affecting systemic energy metabolism and is produced by a number of tissues including muscle and is associated with age and cancer-related sarcopenia, muscle atrophy in mice, cardiovascular disease, chronic inflammatory and autoimmune disease, immune and viral myositis, mitochondrial myopathy, exercise-dependent lipolysis, and anorexia. Other mitochondrial candidate biomarkers for immune and autoimmune diseases include N-formyl methionyl peptides (fMET), 8-hydroxy-2′-deoxyguanosine (8-OhdG), and MT-ND6. fMET modified mitochondrial proteins act as potent activators of the highly expressed neutrophil formyl peptide receptor 1 (FPR1). FPR1 binding promotes neutrophil chemotaxis, neutrophil activation, and NET formation. 8-hydroxy-2′-deoxyguanosine (8-OhdG) or 8-oxo-7,8-dihydro-2′-deoxyguanosine (8-oxodG) is a major form of free radical-induced oxidative lesions in mitochondrial and nuclear DNA, making it useful as a biomarker for conditions resulting in oxidative stress or in cancer. MT-ND6 is a component of the mitochondrial membrane respiratory chain NADH dehydrogenase (Complex I) and therefore involved in electron transport. MT-ND6 has been associated with mitochondrial myopathy and a form of spinal muscular atrophy.

The inventors have advantageously identified a combination of DNA-associated proteins involved in neutrophil function (for e.g., NETs-associated proteins) and mitochondrial proteins (for e.g., GDF-15) as candidate biomarkers for the diagnosis, treatment, and prognosis of FSHD. Since GDF-15 is defined as a cytokine in general, and a myokine more specifically, it is of a different class of molecule from proteins involved in neutrophil function and may help to stratify patients. The inventors also propose multiplexed ELISA to detect such candidate biomarkers, for e.g., S100A8 with the neutrophil and/or GDF-15 biomarker in biological samples obtained from subjects and detection of each of these biomarkers (NE-DNA, MPO-DNA, Calprotectin and GDF-15 or other mitochondrial proteins), either separately and/or in combination with each other, as a diagnostic, monitoring, predictive, and prognostic tool for FSHD.

Accordingly, in an aspect, the disclosure provides a method for diagnosing or detecting Facioscapulohumeral Dystrophy (FSHD) in a subject, the method comprising: obtaining one or more biological samples from the human subject, measuring/detecting an overall expression pattern or expression level of at least one NETs-associated protein in the one or more biological samples obtained from the subject, and comparing the overall expression pattern or expression level of the at least one NETs-associated protein from the one or more biological samples of the subject with the overall expression pattern or level of the at least one NETs-associated protein from a reference sample. In some embodiments, the at least one NETs-associated protein is linked to neutrophil function. In some embodiments, the at least one NETs-associated protein is selected from of at least one of Neutrophil Elastase-DNA (NE-DNA), Myeloperoxidase-DNA (MPO-DNA), and calprotectin. In an embodiment, the reference sample is a biological sample obtained from a normal subject, wherein the normal subject is a subject not suffering from or at risk for FSHD. In some embodiments, detecting an expression or overexpression of the at least one neutrophil extracellular traps (NETs)-associated protein, relative to the expression of the at least one neutrophil extracellular traps NETs-associated protein in the reference sample is indicative of FSHD. In some embodiments, the reference sample is a reference standard sample with known levels of the at least one NETs-associated protein.

In some embodiments, the biological sample is selected from blood, serum, plasma, synovial fluid, bronchoalveolar lavage, spinal fluid, saliva, and the like. In some embodiments, the biological sample from the subject comprises blood, serum, plasma, synovial fluid, bronchoalveolar lavage, spinal fluid, saliva, and the like including any bodily fluid that is likely to contain circulating NETs-associated proteins.

In some embodiments, the reference biological sample is selected from blood, serum, plasma, synovial fluid, bronchoalveolar lavage, spinal fluid, saliva, and the like. In some embodiments, the biological sample from the subject comprises blood, serum, plasma, synovial fluid, bronchoalveolar lavage, spinal fluid, saliva, and the like including any bodily fluid that is likely to contain circulating NETs-associated proteins.

In some embodiments, the detecting an overall expression pattern or expression level of NETs-associated proteins comprises detecting the overall expression pattern or expression level of at least one of Neutrophil Elastase-DNA (NE-DNA), Myeloperoxidase-DNA (MPO-DNA), and calprotectin.

In some embodiments, the method of detecting the presence of the at least one neutrophil extracellular traps (NETs)-associated protein in a biological sample can be an element that is combined with other assays or can be performed alone to detect or monitor FSHD (such as during the course of treatment).

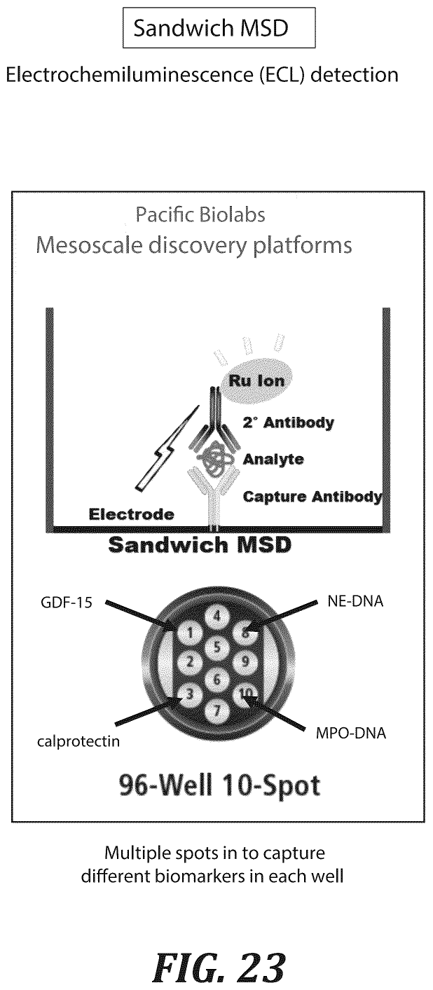

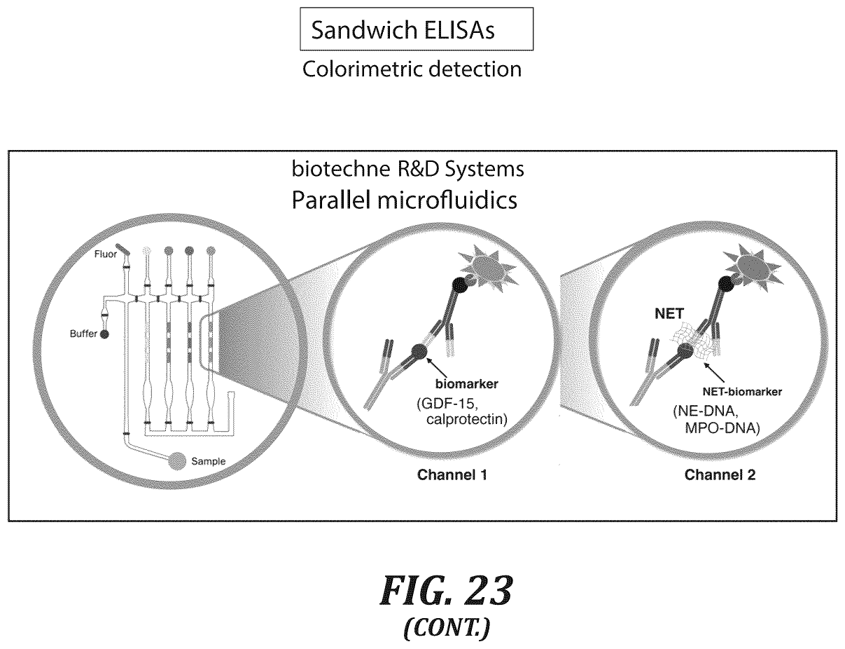

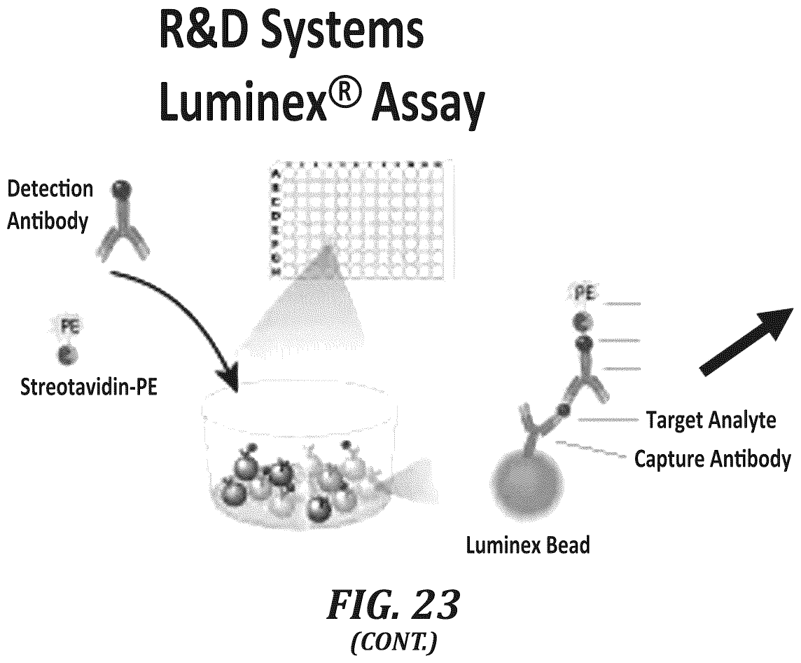

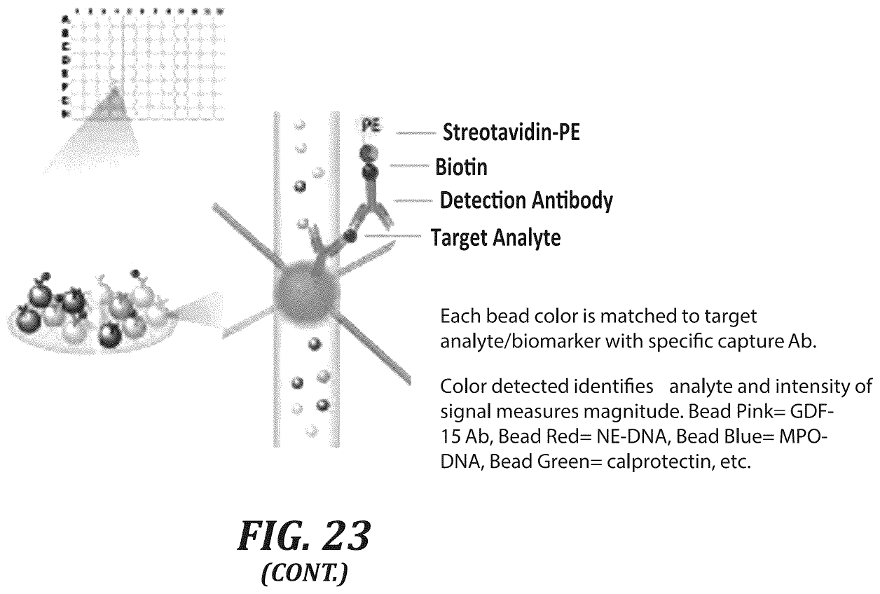

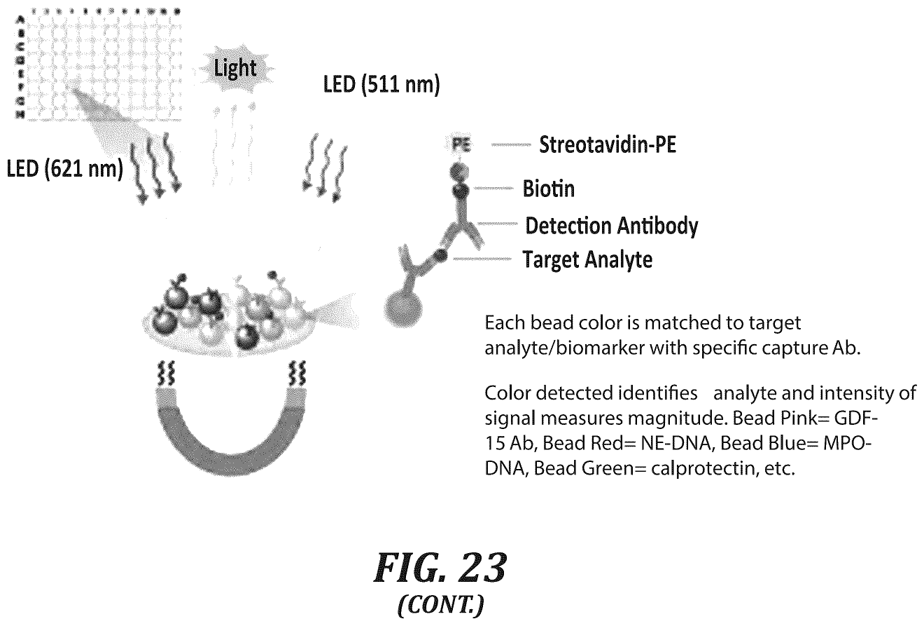

Methods for detecting one or more of the biomarkers disclosed herein include using multiplexing NET-associated protein detection systems. Exemplary detection systems include but are not limited to either a combination of ELISAs built on (i) magnetic beads (for example, Luminex® Assay R&D Systems) to detect both NETs and neutrophil-associated proteins and GDF-15, or (ii) a microfluidic-based system that allows parallel single analyte detection as a quantitative, multi-analyte immunoassay platform (for example, biotechne R&D Systems, SimplePlex™ assay), or (iii) electrochemiluminescence (ECLA) based mesoscale discovery platforms (for example, Pacific Biolabs, ECLA MSD). See FIG. 23.

NETs-associated proteins typically comprise nucleic acids and a combination of certain proteins/enzymes such as myeloperoxidase (MPO), neutrophil elastase (NE), and citrullinated histones. Accordingly, in some embodiments, the NETs-associated protein being detected minimally comprises a complex myeloperoxidase (MPO) and nucleic acid, a complex of neutrophil elastase (NE) and nucleic acid, and/or a complex of citrullinated histones and DNA. An Enzyme Linked Immunosorbent Assay (ELISA) has been previously used to quantitate/detect neutrophil derived enzymes in plasma isolated from blood to look for a relationship between immune and autoimmune disease and the production of NETs-associated proteins in the bloodstream of patients. See U.S. Patent Pub. No. 20210215692 incorporated herein by reference. This assay incorporates the dual recognition of the two elements of NETs-associated proteins, e.g., a protein component, such as myeloperoxidase (MPO), neutrophil elastase (NE) and/or citrullinated histones, and DNA. An important benefit of this assay is the dual recognition of two components of NETs-associated proteins, increasing the specificity of the assay.

In some embodiments, the method of detecting the presence of the at least one NETs-associated protein in a biological sample comprises: contacting the biological sample with a first capture affinity reagent that binds to the at least one NETs-associated protein at a first epitope; contacting the biological sample with a first detection affinity reagent that binds to the at least one NETs-associated protein at a second epitope; and detecting the binding of the first detection affinity reagent to the captured at least one NETs-associated protein. A detected binding of the detectably labeled affinity reagent to the captured at least one NETs-associated protein indicates the presence of the at least one NETs-associated protein in the biological sample.

The detection can be carried out in any acceptable assay format that can differentiate and quantify the detectable labels in the sample. For example, in some embodiments, the binding of the first affinity reagent and binding of the second affinity reagent are detected with flow cytometry, fluorescence microscopy, ImageStream, fluorimetry, or any other appropriate technique that is routinely practiced in the art that is based on imaging colored/labeled cells/particles.

In some embodiments, the capture affinity reagent is immobilized on a solid substrate, such as a well surface or a particle.

In some embodiments, the first epitope is on the protein, for e.g., MPO, NE, or the citrullinated histone within the NETs-associated protein complex, whereas the second epitope comprises the double stranded DNA on the NETs-associated protein complex. Alternatively, it will be understood that the first epitope can comprise the double stranded DNA of the NETs-associated protein complex, whereas the second epitope is on the MPO, NE, or citrullinated histone on the NETs-associated protein complex.

An exemplary, non-limiting affinity reagent that binds to an epitope on MPO is an anti-human MPO antibody (Biorad, #0400-0002), which is encompassed in this disclosure. An exemplary, non-limiting affinity reagent that binds to DNA is an anti-dsDNA antibody (Roche, #11544675001). Other exemplary affinity reagents that bind to dsDNA are labeled dyes known to bind to the dsDNA, for example Sytox-Green, Pico-Green, and the like. Such dyes are encompassed by the disclosure as affinity reagents that bind to dsDNA epitope in a NETs-associated protein. An exemplary, non-limiting affinity reagent that binds to NE is an anti-neutrophil elastase antibody (Calbioshem, #481001).

In some embodiments, the first detection affinity reagent is detectably labeled. Detectable labels, such as fluorescent labels are described above. Alternatively, a detectable label can be configured to emit a detectable signal upon action on a substrate, such as with horseradish peroxidase. Appropriate detectable labels are well-understood in the art and can be implemented into the disclosed method by persons of ordinary skill in the art.

In some embodiments, the method further comprises contacting the sample with a second detection affinity reagent that specifically binds to the first detection affinity reagent. In such embodiments, the second detection reagent has a detectable label and serves to provide a detectable signal on the bound and/or captured NETs-associated protein.

In any embodiment, the capture affinity reagent, the first detection reagent, and/or the second detection affinity reagent can independently comprise an antibody, or a fragment or a derivative thereof, as described herein.

In some embodiments, the biological sample is selected from blood, serum, plasma, synovial fluid, bronchoalveolar lavage, spinal fluid, saliva, and the like. In some embodiments, the biological sample from the subject comprises blood, serum, plasma, synovial fluid, bronchoalveolar lavage, spinal fluid, saliva, and the like including any bodily fluid that is likely to contain circulating NETs.

In some embodiments, the detected presence of the at least one NETs-associated protein in the biological sample indicates the subject has or is at elevated risk of having an inflammatory or autoimmune disease. In some embodiments, detection of an overexpression of the at least one NETs-associated protein in the subject indicates the relative severity or activity of FSHD. Overexpression of NETs can be determined by comparing the detected level to reference standard levels. Such reference standard levels can be determined from biological samples obtained from one or more individuals or subjects without FSHD (e.g., from the same species as the subject) and/or from reference standard samples with known levels of NETs-associated proteins. In some embodiments, the known levels of the NETs-associated proteins are associated with disease indication, activity, progression, or severity. For example, certain concentrations of a NETs-associated protein, that may overlap levels in control samples, could be shown to indicate a subject's tendency to become more affected in a shorter period of time to predict progression. This activity could be established by following subject's samples collected over periods of time in the disease course necessary to establish this phenomenon. In another embodiment the method can be incorporated into a method of monitoring FSHD over a period of time. In some embodiments, the period of time can include the period of administration of therapeutic intervention and/or the period following the administration of the therapeutic intervention for FSHD. In another embodiment treatment responses could define concentration ranges of NET-associated protein in subjects that correspond to a favorable response to treatment.

In some embodiments, the detection of overexpression of the at least one NETs-associated protein in the biological sample indicates the subject has FSHD. In some embodiments, the indicated presence/expression of the at least one NETs-associated protein in the biological sample indicates the subject has an increased risk of developing FSHD.

The method of detection of the of the at least one NETs-associated protein described herein can be conducted alone or in combination with assays for other biomarkers.

In some embodiments, the method further comprises detecting the level of expression of at least one mitochondrial protein and comparing it to a level of expression of the at least one mitochondrial protein in the one or more biological samples obtained from the normal subject or in the reference standard sample.

In some embodiments, the at least one mitochondrial protein is selected from at least one of N-formyl methionyl peptides (fMET), growth and differentiation factor 15 (GDF-15), 8-hydroxy-2′-deoxyguanosine (8-OHdG), and MT-ND6 protein. In some embodiments, the at least one mitochondrial protein is growth and differentiation factor 15 (GDF-15).

Accordingly, in another aspect the disclosure provides a method for diagnosing or detecting Facioscapulohumeral Dystrophy (FSHD) in a subject and/or identifying a subject suffering from or at risk of developing FSHD, the method comprising: obtaining one or more biological samples from the human subject, measuring/detecting an overall expression pattern or expression level of at least one neutrophil extracellular traps (NETs)-associated protein in the one or more biological samples obtained from the subject, measuring/detecting an overall expression pattern or expression level of at least one mitochondrial protein; comparing the overall expression pattern or expression level of the at least one NETs-associated protein and the overall expression pattern or expression level of the at least one mitochondrial protein from the one or more biological samples of the subject with an overall expression pattern or level of the at least one NETs-associated protein and an overall expression pattern or expression level of the at least one mitochondrial protein from a reference sample. In some embodiments, detecting an expression or overexpression of the at least one NETs-associated protein and the at least one mitochondrial protein, relative to the expression of the at least one NETs-associated protein and the at least one mitochondrial protein in the reference sample is indicative of FSHD or a risk of developing FSHD in the subject.

In some embodiments, the reference sample comprises a reference biological sample. In some embodiments, the reference biological sample is obtained from a subject not suffering from FSHD and/or at no risk of developing FSHD. In some embodiments, the reference sample is a reference standard sample with known levels of the at least one NETs-associated protein and/or the at least one mitochondrial protein.

In some embodiments, the detecting an overall expression pattern or expression level of the at least one NETs-associated protein comprises detecting the overall expression pattern or expression level of at least one of Neutrophil Elastase-DNA (NE-DNA), Myeloperoxidase-DNA (MPO-DNA), and calprotectin.

In some embodiments, the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein. In some embodiments, the mitochondrial protein is growth and differentiation factor 15 (GDF-15).

In some embodiments, an expression or overexpression of Neutrophil Elastase-DNA (NE-DNA) and GDF-15 relative to the reference sample identifies the subject as suffering from, or at risk for developing FSHD.

In some embodiments, an expression or overexpression of Myeloperoxidase-DNA (MPO-DNA) and GDF-15 relative to the reference sample identifies the subject as suffering from, or at risk for developing FSHD.

In some embodiments, the method comprises: contacting the biological sample with a first group of capture affinity reagents that bind to one or more components of the at least one NETs-associated protein at a first epitope and contacting the biological sample with a first group of detection affinity reagents that bind to the one or more components of the at least one NETs-associated protein at a second epitope. In some embodiments, the first group of capture affinity reagents are specific for and bind to the at least one NETs-associated protein at the first epitope. In some embodiments, the second epitope of the at least one NETs-associated protein is a binding site on a double stranded DNA. In some embodiments, the first group of detection affinity reagents are specific for and bind to the double-stranded DNA.

In some embodiments, the method further comprises contacting the biological sample with a second group of capture affinity reagents that bind to a first epitope of the at least one mitochondrial protein; and a second group of detection affinity reagents that bind to an epitope of the second group of capture affinity reagents.

In some embodiments, the method further comprises contacting the biological sample with a third group of detection affinity reagents that specifically bind to the first group of detection affinity reagents and/or an epitope on the first group of capture affinity reagents. In such embodiments, the third group of detection reagents have detectable labels and serve to provide a detectable signal on the at least one bound and immobilized NETs-associated protein.

In some embodiment, the method further comprises contacting the biological sample with a fourth group of detection affinity reagents that specifically binds to the second group of detection affinity reagent. In such embodiments, the fourth group of detection reagents have detectable labels and serve to provide a detectable signal on the at least one bound and immobilized mitochondrial protein.

The detection can be carried out in any acceptable assay format that can differentiate and quantify the detectable labels in the sample. For example, in some embodiments, the binding of the first affinity reagent and binding of the second affinity reagent are detected with flow cytometry, fluorescence microscopy, ImageStream, fluorimetry, or any other appropriate technique that is routinely practiced in the art that is based on imaging colored/labeled cells/particles.

The method of detection of the at least one NETs-associated protein and/or the at least one mitochondrial protein described herein can be conducted alone or in combination with assays for other biomarkers.

For example, interleukin-6 (IL-6) concentrations in patient sera have been correlated with clinical severity scores (Spearman r=0.5), Vignos and Brooke functional scores assessing functional impairment of upper and lower limbs, respectively (Spearman r=0.47 and 0.3, respectively), MMT (manual muscle testing)(Spearman r=0.38). These scores are similar to correlation of GDF-15 with functional testing disclosed herein (utilizing Pearson for correlation which is more stringent). The difference in the means between IL-6 and controls was more than double, whereas NE-DNA difference from controls was 5-fold greater with significance of p=<0.0001 (but no correlation with function) compared to IL-6 with a significance of p=0.006. Differences in the mean for GDF-15 were ˜30%>than the mean for controls.

The present disclosure contemplates detection of the at least one NETs-associated protein and/or the at least one mitochondrial protein, and optionally IL-6 in a biological sample obtained from a subject.

Thus provided herein is a method for diagnosing or detecting Facioscapulohumeral Dystrophy (FSHD) in a subject and/or identifying a subject suffering from or at risk of developing FSHD, the method comprising: obtaining one or more biological samples from the human subject, measuring/detecting an overall expression pattern or expression level of at least one neutrophil extracellular traps (NETs)-associated protein in the one or more biological samples obtained from the subject, and/or measuring/detecting an overall expression pattern or expression level of at least one mitochondrial protein, and/or measuring/detecting an expression pattern or expression level of IL-6; comparing the overall expression pattern or expression level of the at least one NETs-associated protein, and/or the overall expression pattern or expression level of the at least one mitochondrial protein, and/or the overall expression pattern or expression level of IL-6 in the one or more biological samples of the subject with an overall expression pattern or level of the at least one NETs-associated protein, an overall expression pattern or expression level of the at least one mitochondrial protein, and an overall expression pattern or expression level of IL-6, respectively from a reference sample.

In some embodiments, the at least one NETs-associated protein comprises a first FSHD biomarker. In some embodiments, the at least one mitochondrial protein comprises a second FSHD biomarker. In some embodiments the at least one NETs-associated protein is selected from Neutrophil Elastase-DNA (NE-DNA), Myeloperoxidase-DNA (MPO-DNA), and calprotectin. In some embodiments, the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

In some embodiments, a determination is made that the subject from which the biological sample was obtained has FSHD, or has an increased risk of developing FSHD, when a first FSHD biomarker disclosed herein is found to be expressed at an increased level of at least 2-fold or greater (i.e., at least 3-fold, at least 4-fold, at least 5-fold, at least 8-fold, at least 10-fold, at least 12-fold, at least 16-fold or greater) in the biological sample (e.g., blood plasma) as compared to the expression of the first FSHD biomarker in a control or reference sample (e.g., blood plasma obtained from a normal subject without FSHD). In some embodiments, the first FSHD biomarker comprises at least one of Neutrophil Elastase-DNA (NE-DNA), Myeloperoxidase-DNA (MPO-DNA), or calprotectin. In some embodiments, the determination is made that the subject from which the biological sample was obtained has FSHD, or has an increased risk of developing FSHD, when second FSHD biomarker disclosed herein is found to be expressed at an increased level of at least 2-fold or greater (i.e., at least 3-fold, at least 4-fold, at least 5-fold, at least 8-fold, at least 10-fold, at least 12-fold, at least 16-fold or greater) in the biological sample (e.g., blood plasma) as compared to the expression of the second FSHD biomarker in a control or reference sample (e.g., blood plasma obtained from a normal subject without FSHD). In some embodiments, the second FSHD biomarker is selected from Myeloperoxidase-DNA (MPO-DNA) and GDF-15.

In some embodiments, the determination is made that the subject from which the biological sample was obtained has FSHD, or has an increased risk of developing FSHD, when a third FSHD biomarker disclosed herein is found to be expressed at an increased level of at least 2-fold or greater (i.e., at least 3-fold, at least 4-fold, at least 5-fold, at least 8-fold, at least 10-fold, at least 12-fold, at least 16-fold or greater) in the biological sample (e.g., blood plasma) as compared to the expression of the third FSHD biomarker in a control or reference sample (e.g., blood plasma obtained from a normal subject without FSHD). In some embodiments, the third FSHD biomarker is IL-6.

In an embodiment, the determination is made that the subject from which the biological sample was obtained has FSHD, or has an increased risk of developing FSHD, when a first biomarker and a second biomarker disclosed herein are found to be expressed at an increased level of at least 2-fold or greater (i.e., at least 3-fold, at least 4-fold, at least 5-fold, at least 8-fold, at least 10-fold, at least 12-fold, at least 16-fold or greater) in the biological sample (e.g., blood plasma) as compared to the expression of the first FSHD biomarker and the second FSHD biomarker, respectively, in a control or reference sample (e.g., blood plasma obtained from a normal subject without FSHD).

In an embodiment, the determination is made that the subject from which the biological sample was obtained has FSHD, or has an increased risk of developing FSHD, when a first biomarker and/or a second biomarker and/or a third biomarker disclosed herein are found to be expressed at an increased level of at least 2-fold or greater (i.e., at least 3-fold, at least 4-fold, at least 5-fold, at least 8-fold, at least 10-fold, at least 12-fold, at least 16-fold or greater) in the biological sample (e.g., blood plasma) as compared to the expression of the first FSHD biomarker, the second FSHD biomarker, and the third biomarker, respectively, in a control or reference sample (e.g., blood plasma obtained from a normal subject without FSHD).

In another aspect, the present disclosure provides a method of treating a subject identified as suffering from, or at risk for developing FSHD, the method comprising administering to the subject an effective amount of a therapeutic agent, wherein the subject is identified/determined as suffering from, or at risk of developing FSHD by the methods disclosed herein. In some embodiments, the method comprises: obtaining one or more biological samples from the subject, measuring/detecting an overall expression pattern or expression level of at least one NETs-associated protein in the one or more biological samples obtained from the subject, and comparing the overall expression pattern or expression level of the at least one NETs-associated protein from the one or more biological samples of the subject, with the overall expression pattern or level of the at least one NETs-associated protein from a reference sample, wherein the reference sample is a biological sample obtained from a normal subject, wherein the normal subject is a healthy subject not suffering from or at risk for FSHD, and wherein detecting an expression or overexpression of the at least one NETs-associated protein relative to the reference sample, identifies the subject as suffering from, or at risk for developing FSHD.

In some embodiments, the measuring/detecting an overall expression pattern or level of the at least one NETs-associated protein comprises measuring/detecting overall expression pattern or level of at least one of Neutrophil Elastase-DNA (NE-DNA), Myeloperoxidase-DNA (MPO-DNA), and calprotectin.

In some embodiments, the method comprises: obtaining one or more biological samples from the subject suffering from, or at risk for developing FSHD, measuring/detecting an overall expression pattern or expression level of at least one NETs-associated protein and at least one mitochondrial protein in the one or more biological samples obtained from the subject, and comparing the overall expression pattern or expression level of the at least one NETs-associated protein and the at least one mitochondrial protein from the one or more biological samples of the subject, with the overall expression pattern or level of the at least one NETs-associated protein and the at least one mitochondrial protein from a reference sample, wherein the reference sample is a biological sample obtained from a normal subject, wherein the normal subject is a healthy subject not suffering from or at risk for FSHD, and wherein detecting an expression or overexpression of the at least one NETs-associated protein and the at least one mitochondrial protein relative to the reference sample, identifies the subject as suffering from, or at risk for developing FSHD.

In some embodiments, the measuring/detecting an overall expression pattern or level of the at least one NETs-associated protein comprises measuring/detecting overall expression pattern or level of at least one of Neutrophil Elastase-DNA (NE-DNA), Myeloperoxidase-DNA (MPO-DNA), and calprotectin.

In some embodiments, the at least one mitochondrial protein is selected from N-formyl methionyl peptides (fMET), growth and differentiation factor 15 (GDF-15), 8 hydroxy-2′-deoxyguanosine (8-OHdG), and MT-ND6 protein.

In some embodiments, an expression or overexpression of Neutrophil Elastase-DNA (NE-DNA) and GDF-15 relative to the reference sample identifies the subject as suffering from, or at risk for developing FSHD.

In some embodiments, an expression or overexpression of Myeloperoxidase-DNA (MPO-DNA) and GDF-15 relative to the reference sample identifies the subject as suffering from, or at risk for developing FSHD.

In yet another aspect, the present disclosure also provides a method of monitoring the status of the FSHD in the subject over a period of time. The monitoring includes performing the described steps at multiple time points within a defined period of time to ascertain the status or character of the condition, e.g., whether the condition is stable, progressing, in remission, or changing to other indications, etc. In some embodiments, the defined period of time includes administration of a therapy or other intervention to the subject. The method can assist a care provider to understand the efficacy of the therapy or intervention.

The present disclosure contemplates therapeutic agents that utilize immune cell targeted anti-inflammatory drugs that reduce the stimulation of neutrophils in muscle and other tissues to reduce tissue damage initiated by DUX4 expression. Interventions in aspects of inflammation related to innate immunity to prevent the cascade of tissue damage progression observed in FSHD, are also contemplated. Since GDF-15 can inhibit leukocyte integrin activation to help recovery after myocardial infarction and is a central mediator of immune tolerance, GDF-15 expression in FSHD tissue could act to control muscle immune destruction caused by neutrophils and macrophages to act as a therapy for controlling disease. Gene therapies to manipulate the levels of neutrophil stimulation for prevention of NET formation in tissues and blood and/or for modulating the levels of GDF-15 in tissues are also contemplated. Such therapies may be administered alone or in combination with agents targeting aberrant DUX4 expression or overexpression in FSHD.

Appropriate agents for targeting DUX4 in FSHD are known and are encompassed by this disclosure. Exemplary agents targeting DUX4 expression or overexpression include but are not limited to nucleic acids, encoding any one or more anti-DUX4 antisense agents or RNAi agents targeting DUX4 transcript. Agents such as antisense or RNAi agents as taught herein may without limitation reduce or abolish the production and/or level of DUX4 and/or pre-mRNA and/or mRNA, whereby such agents may be capable of reducing or abolishing the production of aberrantly overexpressed or expressed DUX4 proteins. Also included are pharmaceutical compositions and formulations comprising any one or more anti-DUX4 antisense agent or RNAi agent as taught herein, or an isolated nucleic acid encoding such an agent, a recombinant construct (vector) (preferably an expression construct, expression vector) comprising a nucleic acid encoding such an agent, or a host cell or host organism, one or more pharmaceutically acceptable carriers; and methods for producing said pharmaceutical compositions and formulations, comprising admixing said agent, isolated nucleic acid, construct (vector), host cell or host organism as taught herein with said one or more pharmaceutically acceptable carriers. Other exemplar agents targeting DUX4 include, but are not limited to agents that modifies chromatin or promoter DNA to prevent DUX4 gene expression, such as CRISPR/Cas9, or pharmacological agents that bind DUX4 protein and inhibits its activity.

The methods described here for detecting agents that are byproducts of disease indicate that said agents could be controlled to affect disease. Exemplar agents here could be pharmacological entities such as specific inhibitors of the NET formation process where neutrophil surface proteins are targeted to reduce the potential for neutrophil stimulation that would result in NET formation, such as synthetic antibodies or an embodiment of such that binds NET-related entities, to reduce aspects of the inflammatory process linked to these cells. Another exemplar agent limiting neutrophil stimulation could include, but not be limited to, an anti-citrullinated histone binding agent, such as an inhibitor or biological based inhibitor such as an antibody or form thereof. Therapeutic agents could include agents that bind GDF-15 or other identified myokines or mitochondrial signaling molecules that induce inflammation directly by muscle cells as is the case with GDF-15, including but not limited to, specific pharmacological inhibitors or custom designed and manufactured binding agents, other binding agents such as synthetic antibodies.

The therapeutic agents and/or pharmaceutical compositions of the disclosure may be formulated for essentially any route of administration, such as without limitation, oral administration (such as, e.g., oral ingestion or inhalation), intranasal administration (such as, e.g., intranasal inhalation or intranasal mucosal application), pulmonary (such as, e.g., by inhalation or insufflation of powders or aerosols), parenteral administration (such as, e.g., subcutaneous, intravenous, intra-arterial, intramuscular, intraperitoneal or intrasternal injection or infusion, or intracranial, e.g., intrathecal or intraventricular administration), epidermal and transdermal, or transmucosal (such as, e.g., oral, sublingual, intranasal) administration, topical administration (including inter alia ophthalmic administration), rectal, vaginal or intra-tracheal instillation, and the like. In this way, the therapeutic effects attainable by the methods and compositions of the invention can be, for example, systemic, local, tissue-specific, etc., depending on the specific needs of a given application of the invention.

The routes of administration described are intended only as a guide since a skilled practitioner will be able to determine readily the optimum route of administration and any dosage for any particular animal and condition.

Further, there are several well-known methods of introducing nucleic acids (e.g., antisense and RNAi agents) into animal cells, any of which may be used herein. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors, for example such as taught herein, electroporation, and the like. Other techniques or methods which are suitable for delivering nucleic acid molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable microspheres. Also envisaged is encapsulation of NA in various types of liposomes (immunoliposomes, PEGylated (immuno) liposomes), cationic lipids and polymers, nanoparticles or dendrimers, poly (lactic-Co-Glycolic Acid) polymeric microspheres, implantable drug-releasing biodegradable microspheres, etc; and co-injection of NA with protective agent like the nuclease inhibitor aurintricarboxylic acid. It shall be clear that also a combination of different above-mentioned delivery modes or methods may be used.

Further ways of delivery of nucleic acids such as antisense agents and RNAi agents may employ previously published methods. For example, other methods of delivery of nucleic acids to the nucleus are described in Mann et al. 2001 (Proc Natl Acad Science 98(1): 42-47) and in Gebski et al. 2003 (Human Molecular Genetics 12(15): 1801-1811). A method for introducing a nucleic acid molecule into a cell by way of an expression vector either as naked DNA or complexed to lipid carriers, is described in U.S. Pat. No. 6,806,084.

In another aspect, the present disclosure provided a kit, comprising: a first group of capture affinity reagents that bind to at least one NETs-associated protein at a first epitope, and a first group of detection affinity reagents that bind to the at least one NETs-associated protein at a second epitope. In some embodiments, the first group of capture affinity reagents are specific for and bind to the at least one NETs-associated protein at the first epitope. In some embodiments, the at least one NETs-associated protein is selected from NE-DNA, MPO DNA, and calprotectin.

In some embodiments, the kit further comprises a second group of capture affinity reagents that bind to a first epitope of at least one mitochondrial protein; and a second group of detection affinity reagents that bind to an epitope of the at least one second group of capture affinity reagents. In some embodiments, the second group of capture affinity reagents are specific for and bind to the at least one mitochondrial protein at the first epitope. In some embodiments, the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

In some embodiments, the first group of capture affinity reagents are immobilized on a solid substrate. In some embodiments, the second group of capture affinity reagents are immobilized on a solid substrate.

This aspect of the disclosure encompasses any relevant affinity reagent, such as defined in more detail below. In some embodiments, the one or more affinity reagents are independently an antibody, or an antigen-binding fragment or a derivative thereof. An exemplary, non-limiting affinity reagent that binds to an epitope on MPO is an anti-human MPO antibody (Biorad, #0400-0002), which is encompassed in this disclosure. An exemplary, non-limiting affinity reagent that binds to DNA is an anti-dsDNA antibody (Roche, #11544675001). Other exemplary affinity reagents that bind to dsDNA are labeled dyes known to bind to the dsDNA, for example Sytox-Green, Pico-Green, and the like. Such dyes are encompassed by the disclosure as affinity reagents that bind to dsDNA epitope in a NET. An exemplary, non-limiting affinity reagent that binds to NE is an anti-neutrophil elastase antibody (Calbioshem, #481001).

In some embodiments, the detection affinity reagent is detectably labeled. Detectable labels, such as fluorescent labels are described above. Alternatively, a detectable label can be configured to emit a detectable signal upon action on a substrate, such as with horseradish peroxidase. Appropriate detectable labels are well-understood in the art and can be implemented into the disclosed method by persons of ordinary skill in the art.

In accordance with the foregoing, the disclosure provides several methods, and related compositions and kits, for detection of FSHD. The disclosed methods and related compositions can be integrated into methods of medical intervention. Various aspects of the disclosure are addressed herein.

The above aspects and embodiments are further supported by the following non-limiting examples.

### Example 1

### Mouse Model of FSHD

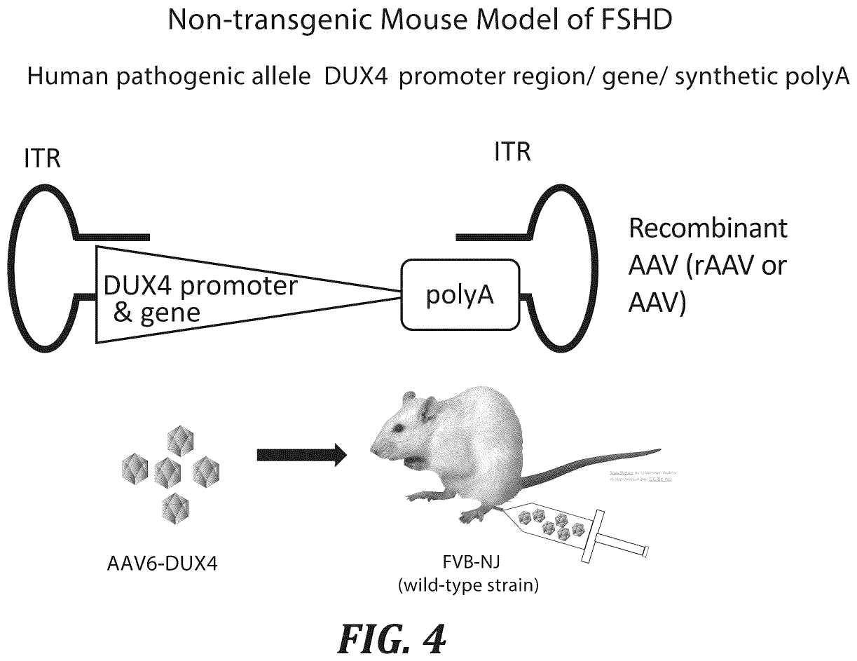

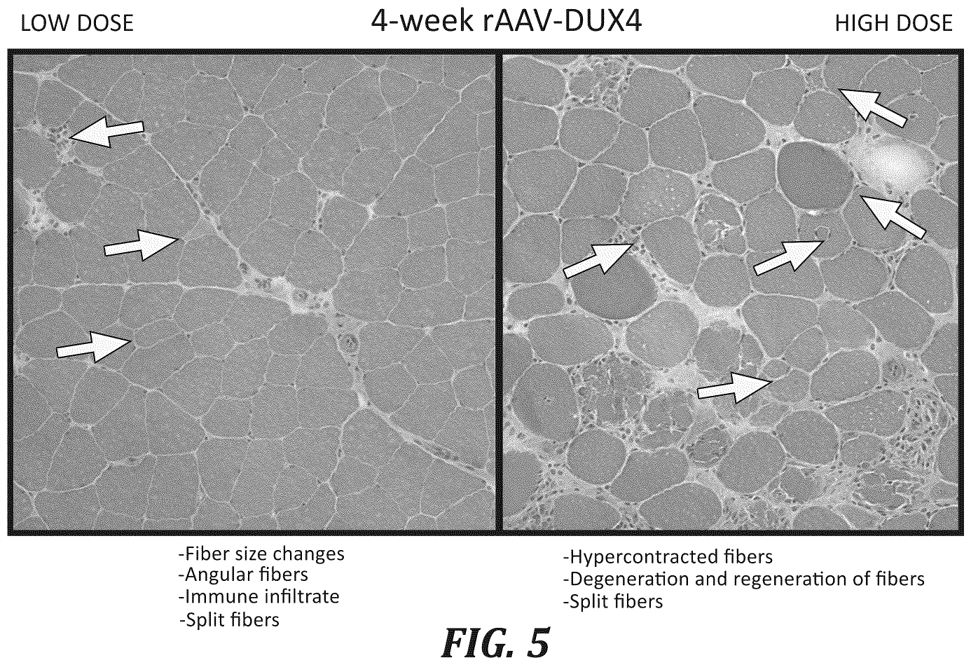

A mouse model of FSHD was generated using vector-mediated delivery of the DUX4 gene to mouse muscle using standard laboratory gene delivery vectors (FIG. 4). DNA Sequences were incorporated from the lambda phage #42 human FSHD clone (L42; pathogenic DUX4 allele A161) containing a DUX4 gene in the last 3.3 kb D4Z4 repeat on chromosome 4q35, followed by a partial repeat, polyadenylation sequence, and containing 2 DNA deletions for vector production and stability. Injection of adeno-associated virus vector serotype 6 (AAV6) carrying this human DUX4 gene and the DUX4 promoter region, referred to as AAV-DUX4 (SEQ ID NO: 1) hereafter, into the tibialis anterior leg muscle of wild type mice resulted in focal expression of DUX4 mRNA and protein. The sites of DUX4 expression also displayed structural cellular changes that indicated damage in the region of DUX4 expression, including fiber size changes with hypercontracted and triangular shaped fibers, immune cell infiltrated fibers associated with dying fibers, centrally and non-peripherally nucleated fibers, and some fat and fibrotic tissue replacement. The AAV-DUX4 mouse model is unique, with DUX4 expression controlled by DUX4 promoter sequences that expresses in a focal pattern like focal damage observed in FSHD muscle (FIG. 5).

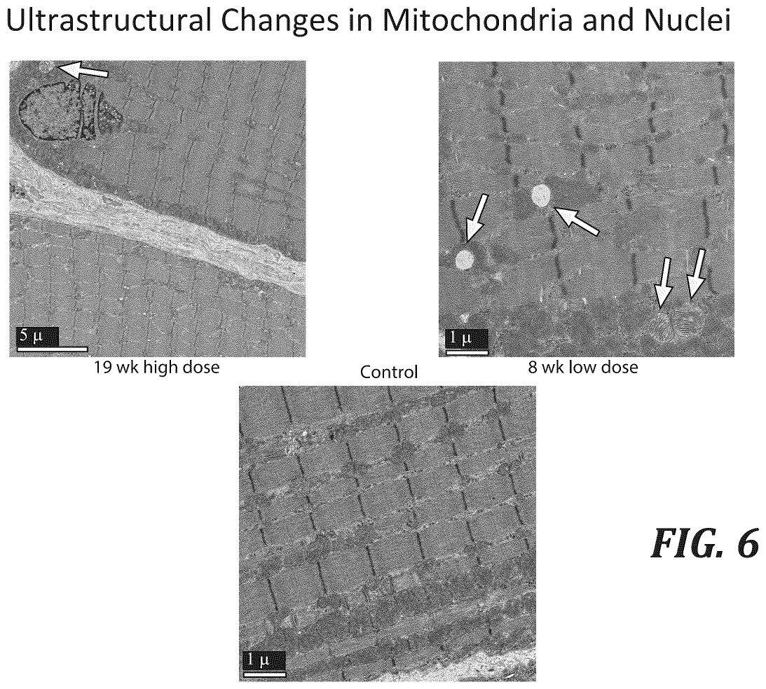

Consistent with oxidative stress effects in FSHD tissue ultrastructural changes observed in muscle from the AAV-DUX4 with electron microscopy include swollen mitochondria with damaged cristae as a precursor of mitophagy (FIG. 6). The mouse model of FSHD was used to interrogate the in vivo disease manifestations and outcomes and their relationship to FSHD to understand underlying disease mechanism. AAV-DUX4 mouse findings will be used to develop candidate FSHD treatments to test in the AAV-DUX4 mouse, as well as other FSHD animal models. Beyond developing candidate therapies, the mouse model of FSHD is useful to study disease related changes that could be tracked easily to evaluate disease progression or stabilization and regression with therapeutic interventions.

### Example 2

### Neutrophil Involvement in FSHD

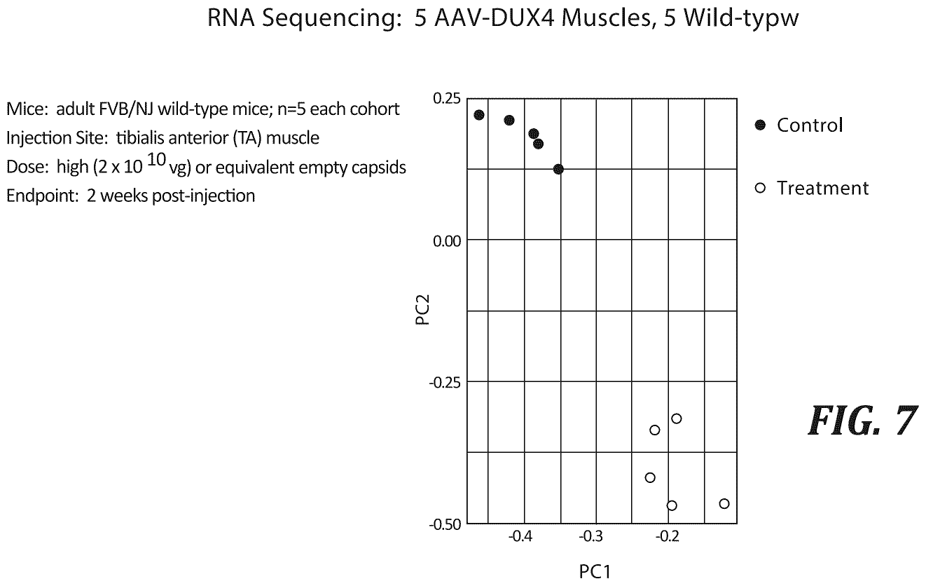

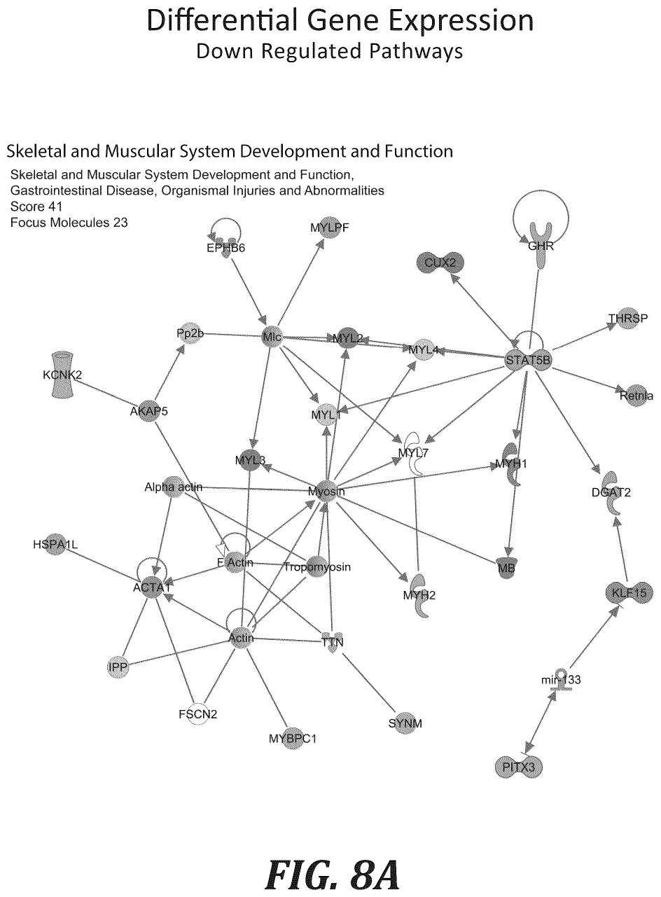

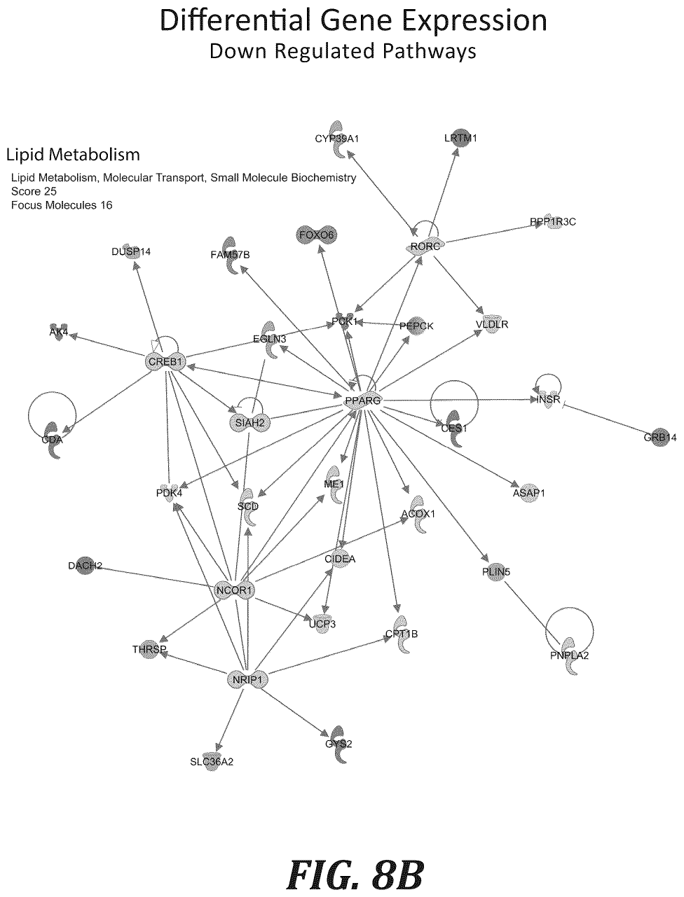

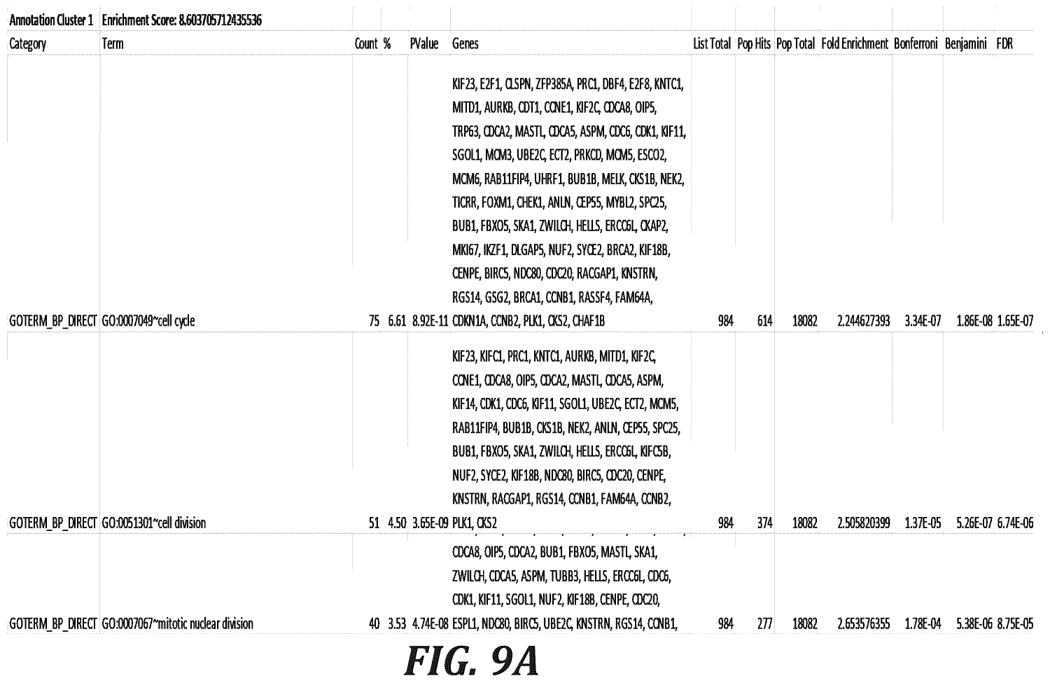

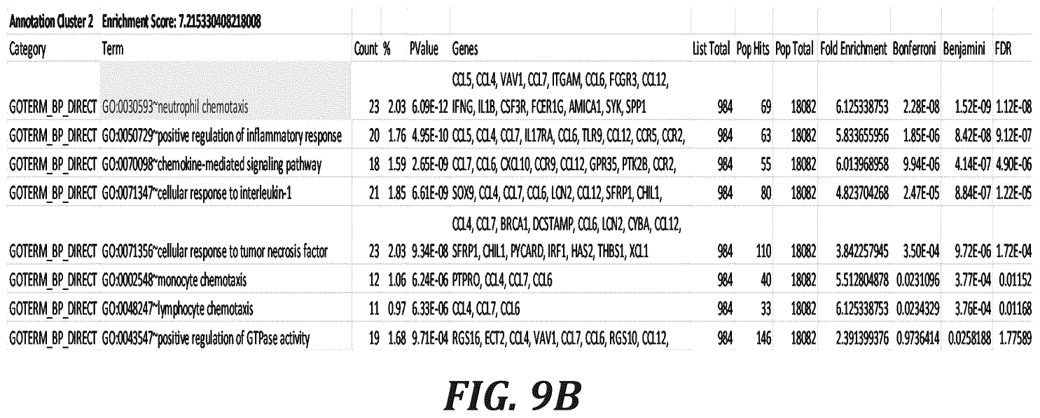

Analysis of physiological response to DUX4 expression in the AAV-DUX4 mouse resulted in identification of a cell in muscle that was implicated the disease process. This link was made through analysis of gene expression changes with analysis of RNA collected from DUX4 expressing muscle used for RNA sequencing. A primary analysis of DUX4-expressing muscle from the AAV-DUX4 mouse was RNA sequencing (RNAseq). Wild-type FBV/NJ mice tibialis anterior (TA) muscles were injected with 2×10¹⁰ vector genomes (vg) of AAV-DUX4 or AAV empty capsid (virions without DNA of equivalent protein content)(n=5 each group). RNA was isolated for sequencing 2 weeks post injection. This time point at this dose produces evidence of focal muscle damage with limited muscle fiber structural changes. Damage is apparent with observation of 1-5 degenerating fibers in several unique regions in a 10 μm H&E stained transverse cryosections of muscle, with accompanying membrane damage and immune cell infiltrates to initiate cell clearance. Principal Component Analysis (PCA) shows a clear distinction between the control and AAV-DUX4 injected samples (FIG. 7). Ingenuity Pathway Analysis (Qiagen) of genes differentially expressed resulted in decreased expression of disease-relevant pathways. Two of the most downregulated pathways are highlighted in FIGS. 8A-8B, including ‘Skeletal and Muscular System Development and Function’ and ‘Lipid Metabolism.’ In the process of disease FSHD muscle weakens, becomes fibrotic, and is eventually replaced with fat cells. The top two Gene Ontology (GO) annotation clusters for biological processes highlight cell cycle and cell division genes upregulated in cluster 1 (FDR 1.65E-07 [GO:00007049] and 6.74E-06 [GO:0051301], respectively) and neutrophil chemotaxis and chemokine signaling in cluster 2 among other immune related processes (FDR 1.12E-08 [GO:0030593] and 4.9E-06 [GO:0070098], respectively) (FIGS. 9A-9B). These results demonstrated that neutrophil activity was altered in FSHD disease.

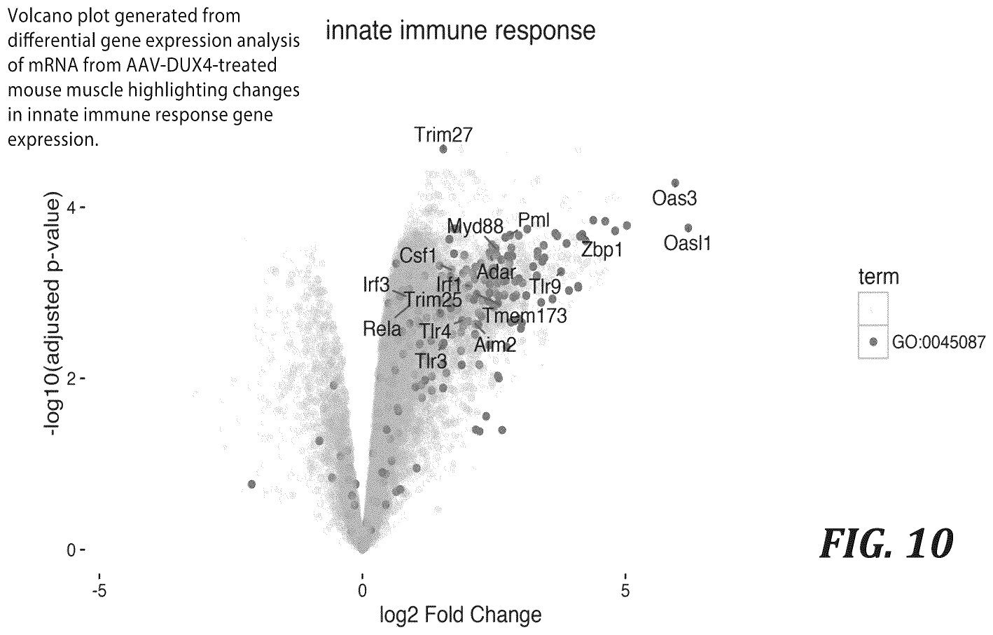

Several lines of research indicated that the innate immune system was involved in FSHD, including an estimation of immune cell increases in ˜30% of biopsied FSHD muscle and many innate immune effectors were seen to be upregulated with AAV-DUX4 administration (FIG. 10). The RNAseq data indicating neutrophil involvement suggested that these cells were signaling the detection of DUX4-related cellular damage. The results from the RNAseq studies both support and extend the understanding of disease changes and strongly suggested that neutrophils could be activated following DUX4 expression.

### Example 3

### NETs as a Biomarker for FSHD

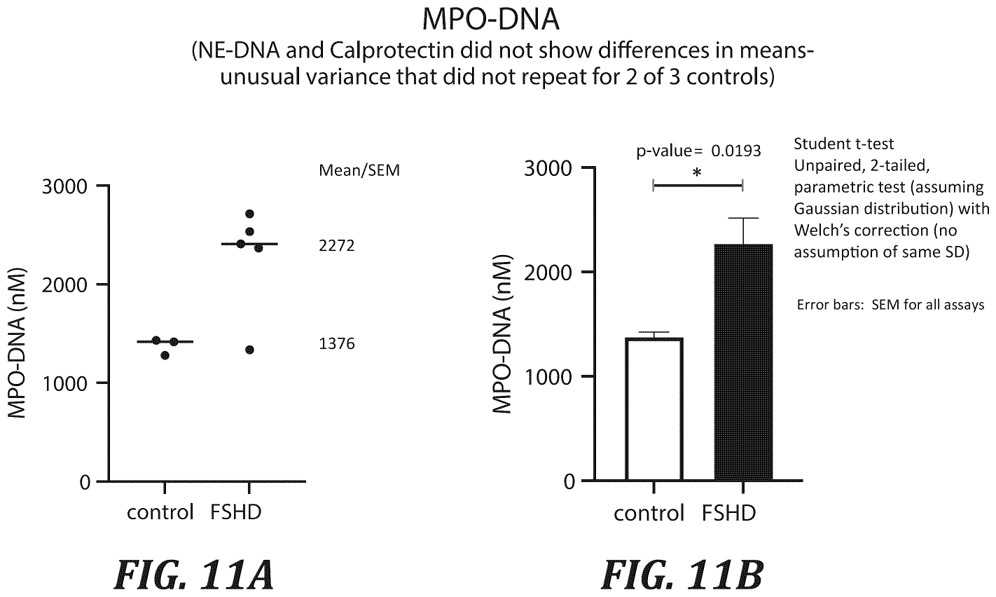

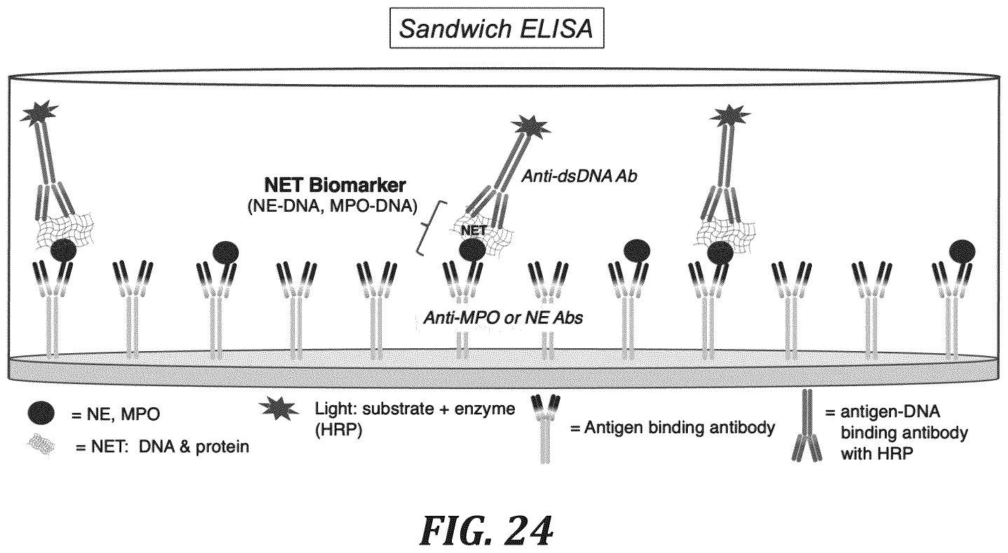

As part of an ongoing study of FSHD muscle tissue (collected by Dr. Leo Wang with UW IRB approval for Joel Chamberlain), patient plasma samples were obtained in preparation for muscle needle biopsy. The purpose of the blood draw was to check for any indication of an imbalance or signs of infection as a prerequisite evaluation before proceeding with the biopsy procedure. 5 FSHD plasma samples (4 different individuals' plasma; 1 repeat blood draw from same patient) and 3 controls were tested by NETs specific ELISA (described in US Patent Publication No. 20210215692A1), which targeted NET-double stranded DNA bound proteins (FIG. 24). Six (6) more samples were evaluated in a second study. Statistical analyses using Student's t-test indicated a significant difference in the means for 1 of 3 proteins assayed for the initial pilot study, which included detection of myeloperoxidase (MPO), neutrophil elastase (NE), and a complex of S100A8/S100A9 (calprotectin) (FIGS. 11A-11B). Detection of dsDNA-bound MPO (MPO-DNA) resulted in a significant difference in the means with Student's t-test (p=0.0193). S100A8 presence in plasma associated with FSHD has been previously reported such that calprotectin, although a complex of 2 proteins, was considered a surrogate positive control, although the complex did not reach any significance in the previous study.

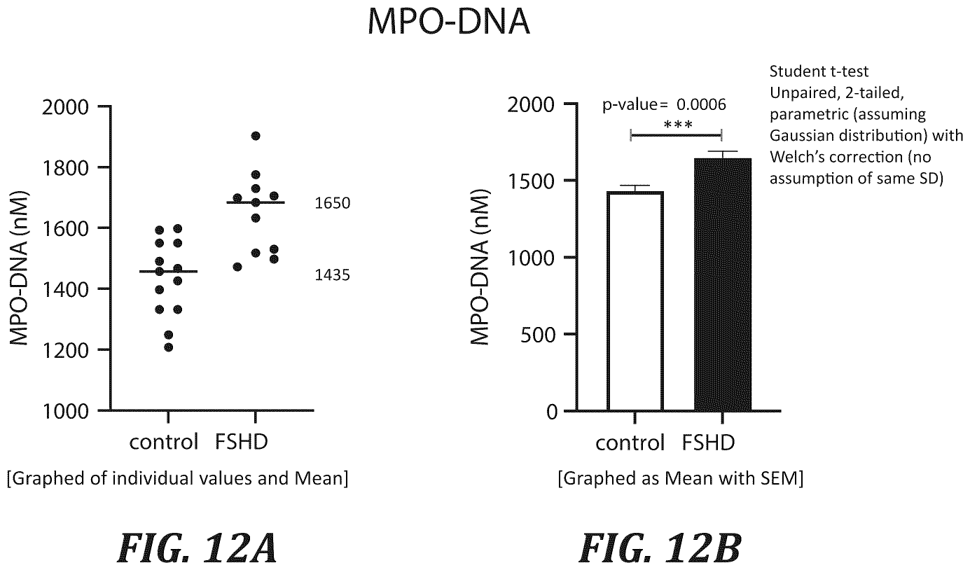

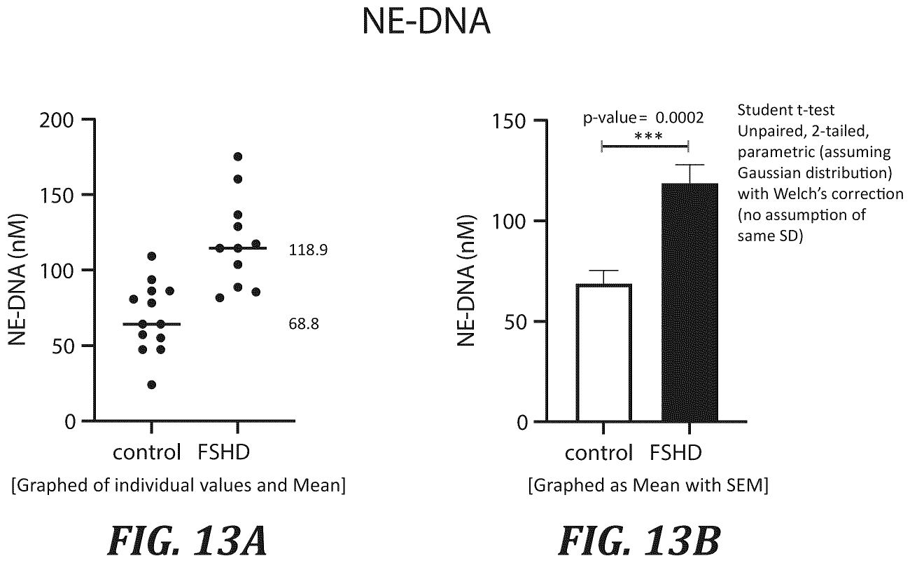

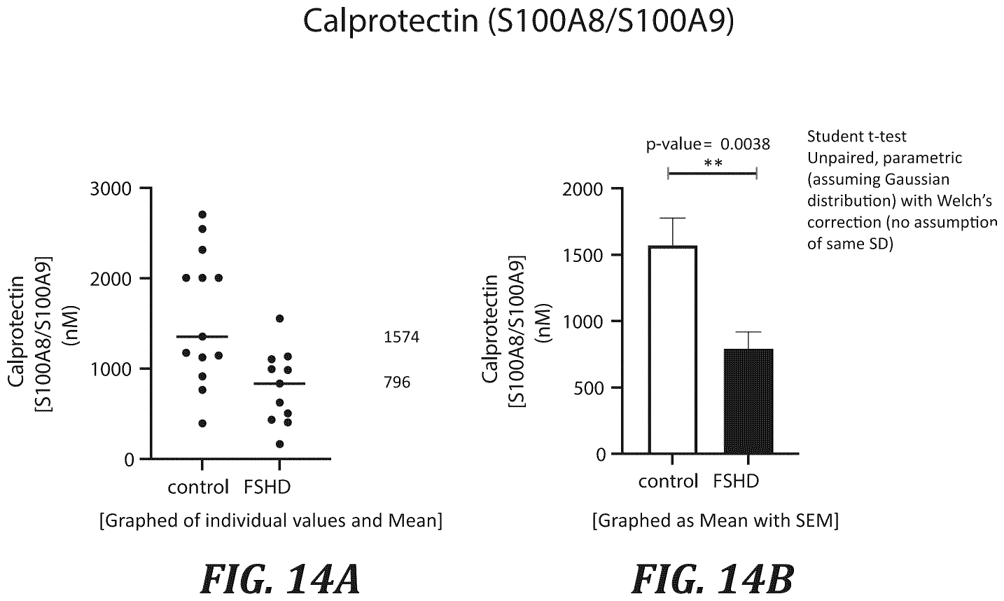

The indication of significance of a change in MPO levels compared to controls motivated a second assay with an additional 6 FSHD samples and 10 additional control plasma samples. Results from this ‘Extended Pilot Study’ with a total of 11 FSHD samples and 13 controls showed a significant difference in the means for all 3 complexes (p=0.0006 MPO (FIGS. 12A-12B), p=0.0002 NE (FIGS. 13A-13B), p=0.0038 calprotectin) (FIGS. 14A-14B). Statistics were carried out using Prism (GraphPad) with the Student's t-test (unpaired, 2-tailed, parametric, assuming Gaussian distribution, with Welch's correction [no assumption of same SD], standard error of the mean, SEM). The findings from the NET-based protein ELISA assay comparing FSHD DNA associated plasma proteins to control DNA associated plasma proteins demonstrate a clear and significant difference in the means with the FSHD samples.

### Example 4

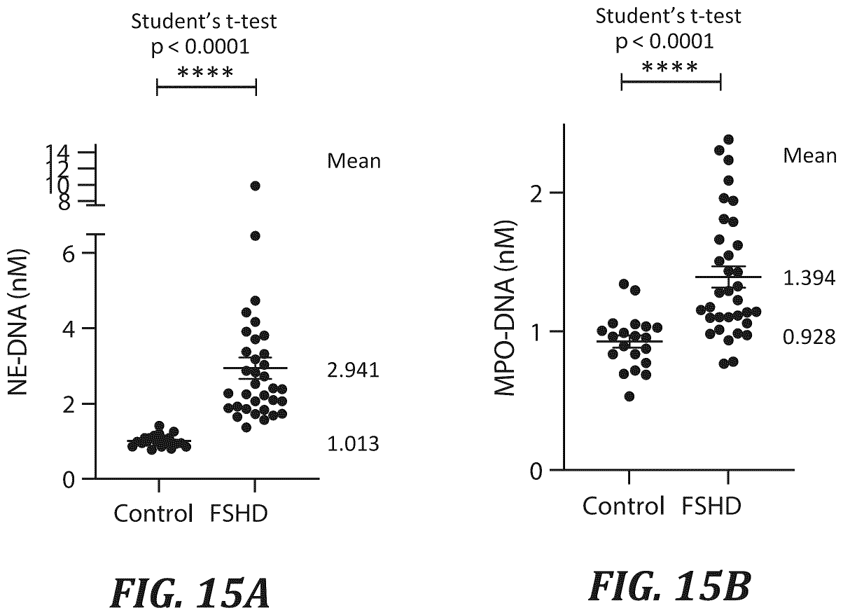

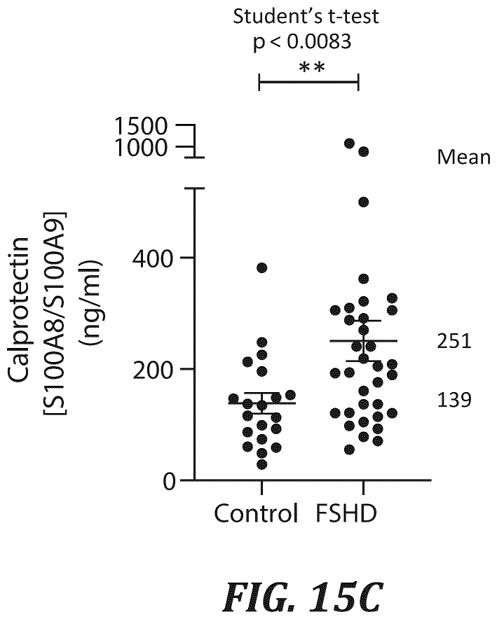

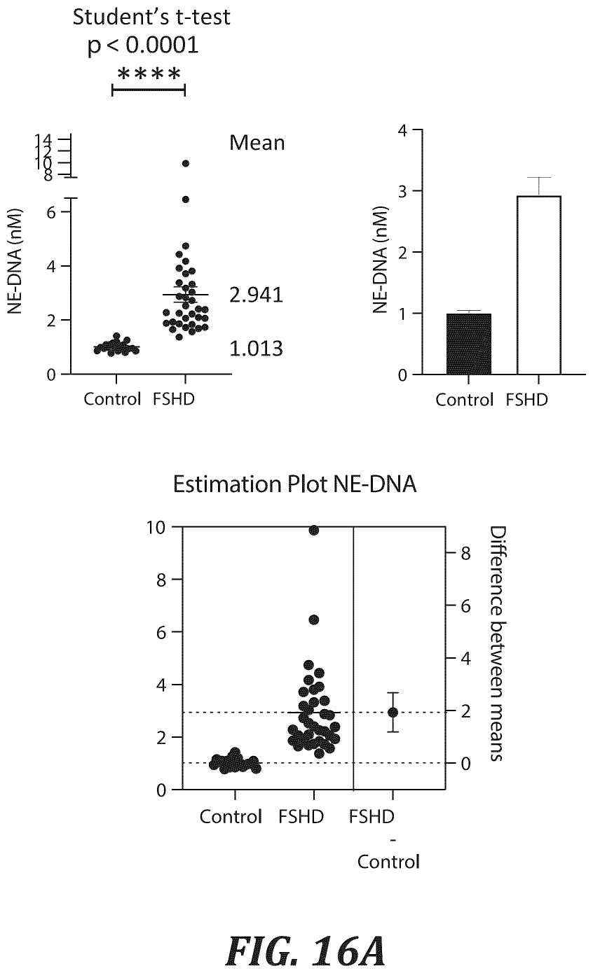

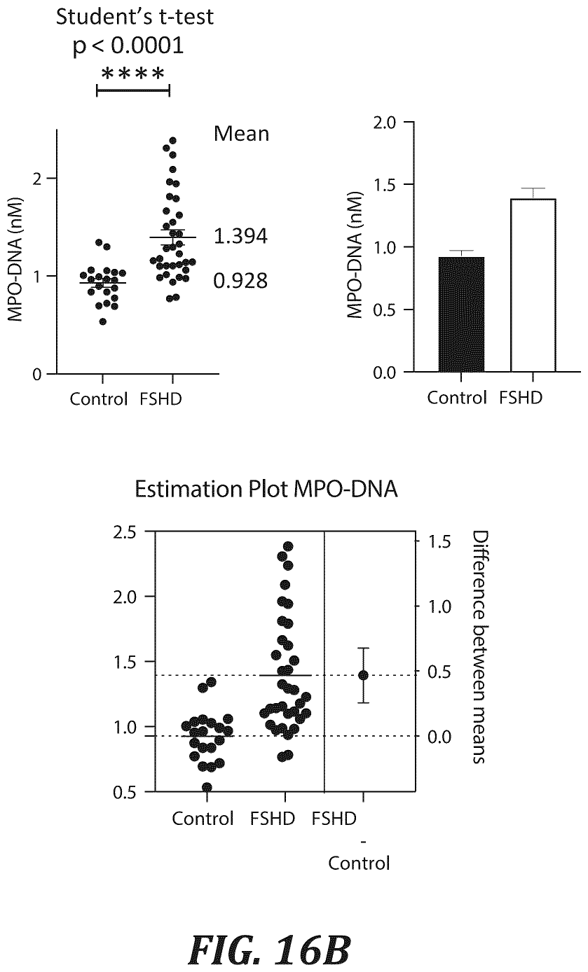

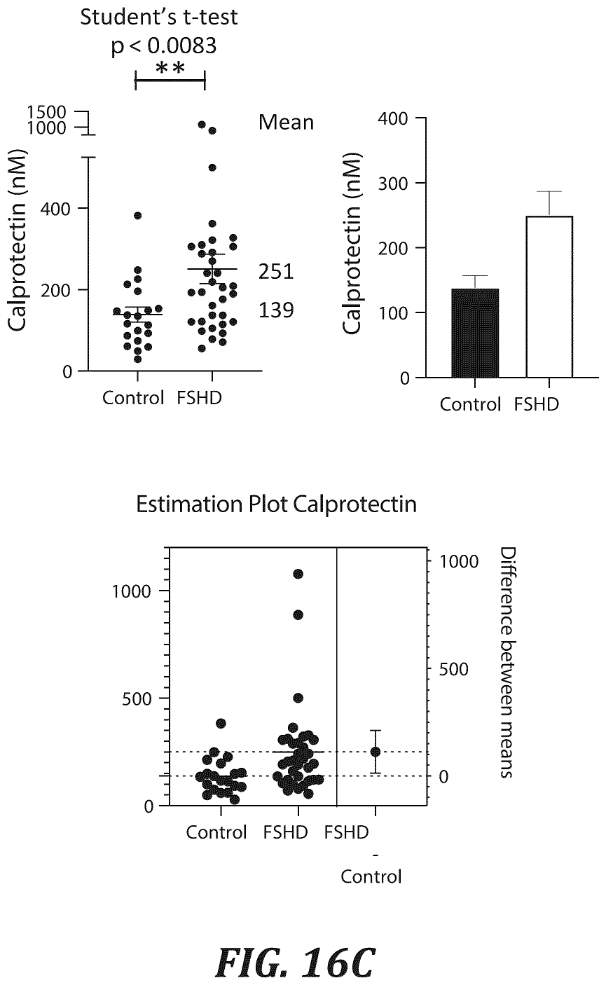

To further power studies to explore the use of MPO-DNA and NE-DNA blood levels as biomarkers for FSHD, samples were obtained from the Wellstone Study collection using patient tissue samples. These samples were collected and curated by clinicians prior to MRI-guided muscle biopsy for use in characterizing disease changes and in developing and tracking biomarkers for FSHD. A wealth of data, including FSHD DNA mutations, subject age, disease severity, and muscle changes, are connected to these samples to support analyses for candidate biomarker performance. 34 FSHD Wellstone plasma samples and 20 adult human control samples were evaluated as part of an Exploratory Study. A high significance for MPO-DNA and NE-DNA (p<0.0001) and lower significance for calprotectin (p=0.0038) (FIGS. 15A-15C) was observed. The greatest difference in the mean in the Student's t test was again for NE-DNA with double the concentration difference (p=0.0001; see plot in FIG. 16A in the group in the Estimation Plot with right y-axis titled ‘Difference between Means’). The controls did not show much variation compared to the FSHD samples which are expected to vary widely due to the wide-ranging extent of involvement of different muscles in the disease. Note that overall concentration values are different from the earlier studies and reflect the concentration relative to a different standard curve values used in this study. The differences in the means for MPO-DNA and calprotectin were significant, p=0.0001 and p=0.0083, respectively, although samples from both groups had overlapping values (FIGS. 16B-16C). The lower values in the FSHD samples may indicate less immune stimulation in these individuals (NETosis level overall that is separate from muscle damage) or reflect a difference in the cells and proteins involved in the immune response not detected with these assays. Overall, the results from this study indicate a strong relationship of NETs as candidate blood biomarkers for FSHD and particularly highlight the potential for NE-DNA with the greatest separation in the means from control samples.

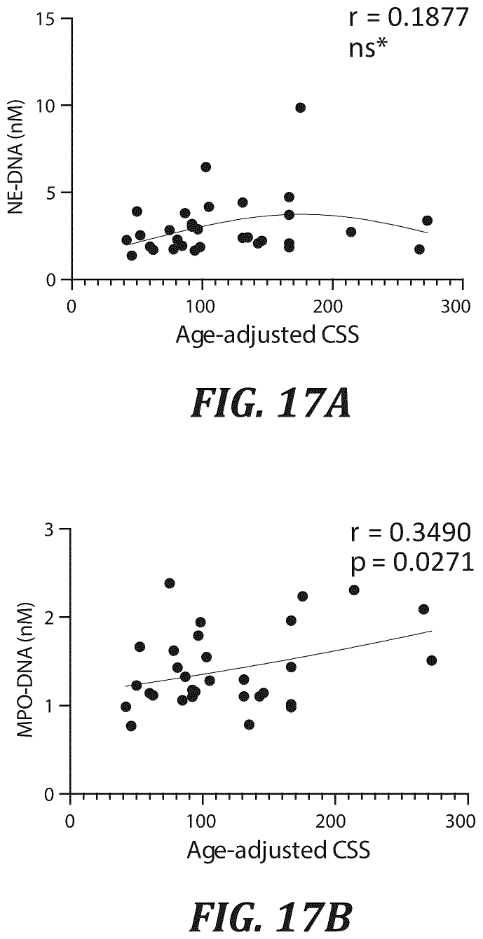

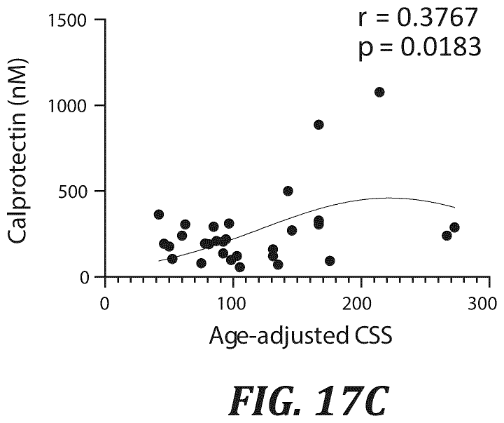

Additional data at a second 12-month time point were collected for individuals in the Wellstone cohort included in the Exploratory Study, which allowed for the evaluation of the predictive value of these assays for disease monitoring with testing of those samples in the future. Specifically, the relationship of age-adjusted clinical severity score (ACSS) for these patients was evaluated compared to the level of each biomarker (FIGS. 17A-17C). Using calculations of Pearson's correlation coefficient, a correlation between ACSS for both MPO-DNA (r=0.3490; p=0.0271) and calprotectin (r=0.3767; p=0.0183) was observed (FIGS. 17B-17C).

### Example 5

### Mitochondrial Proteins as Biomarkers of FSHD

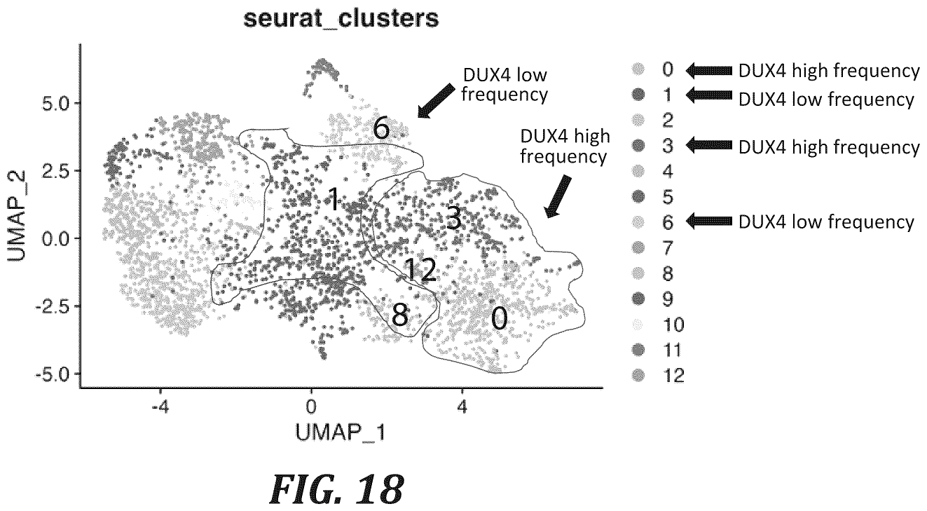

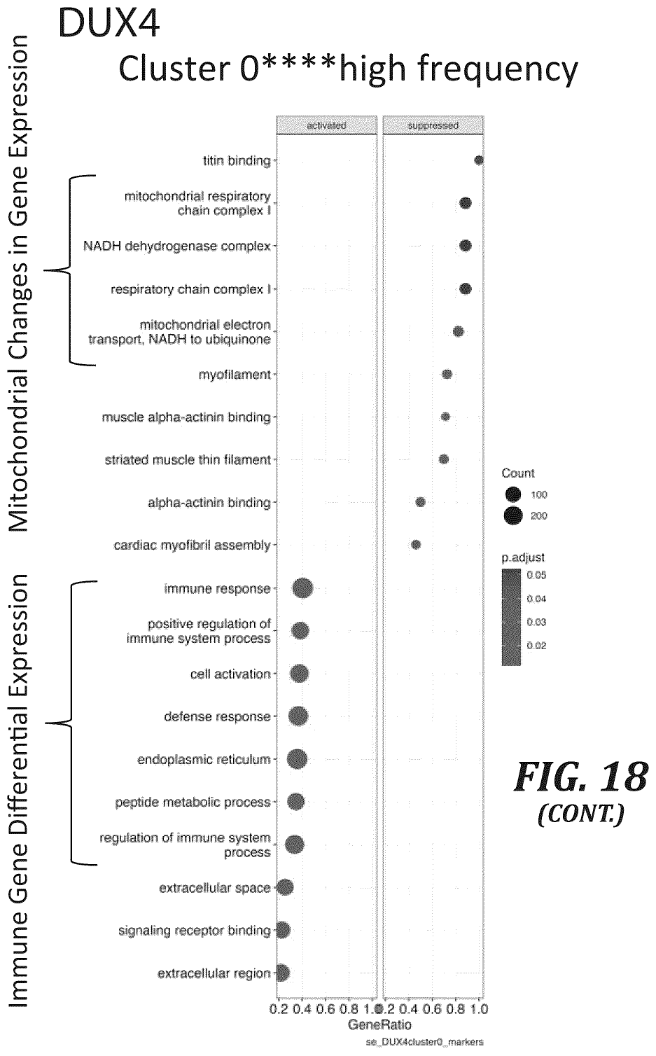

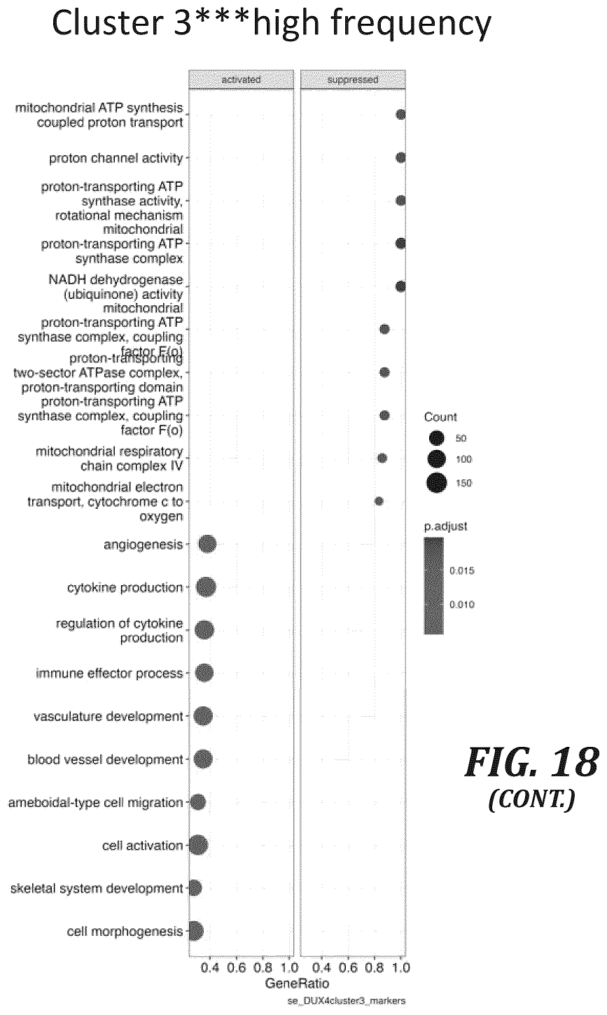

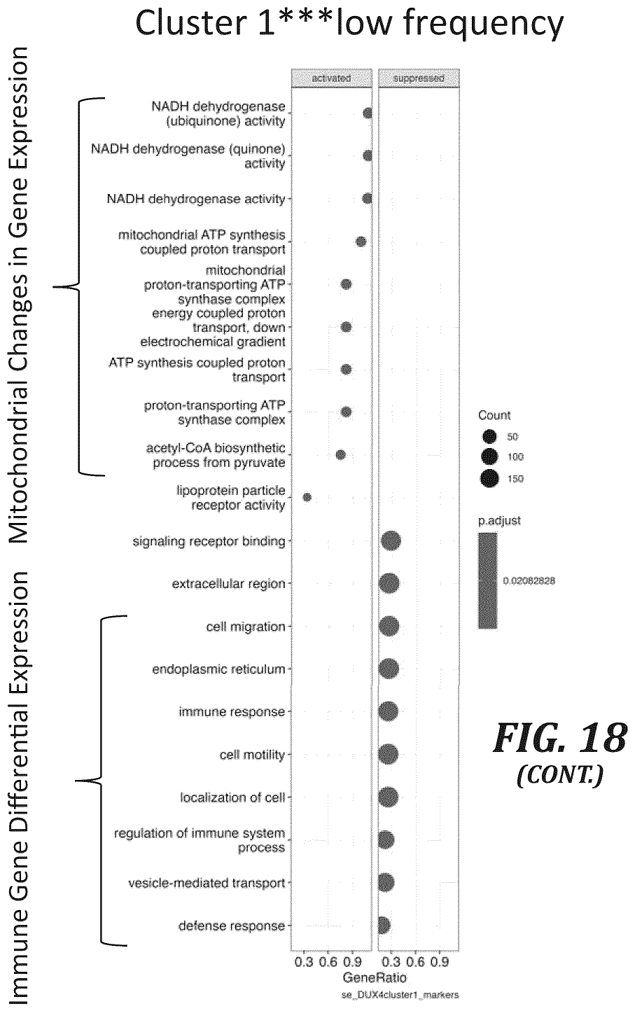

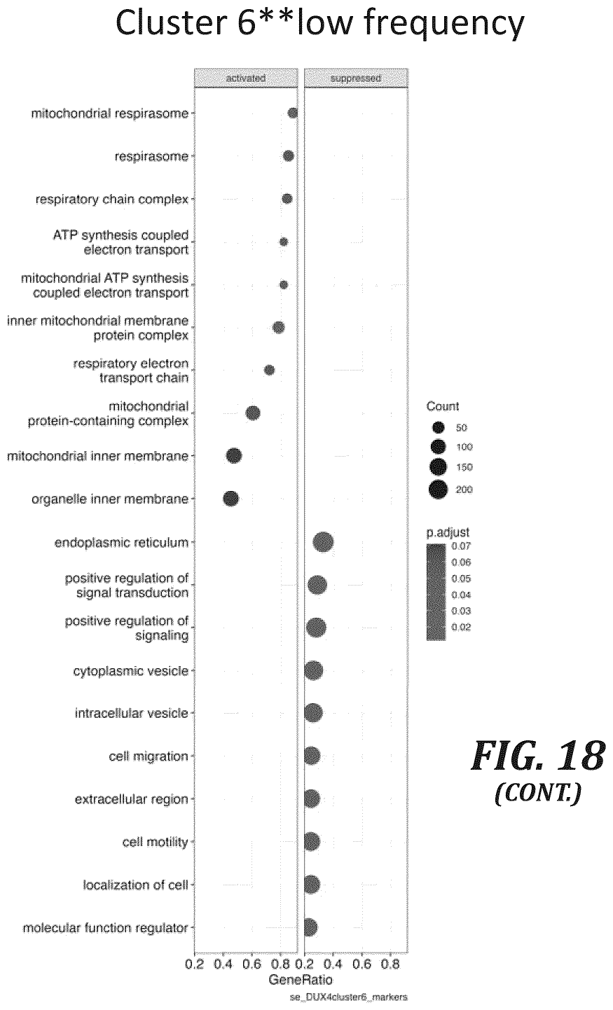

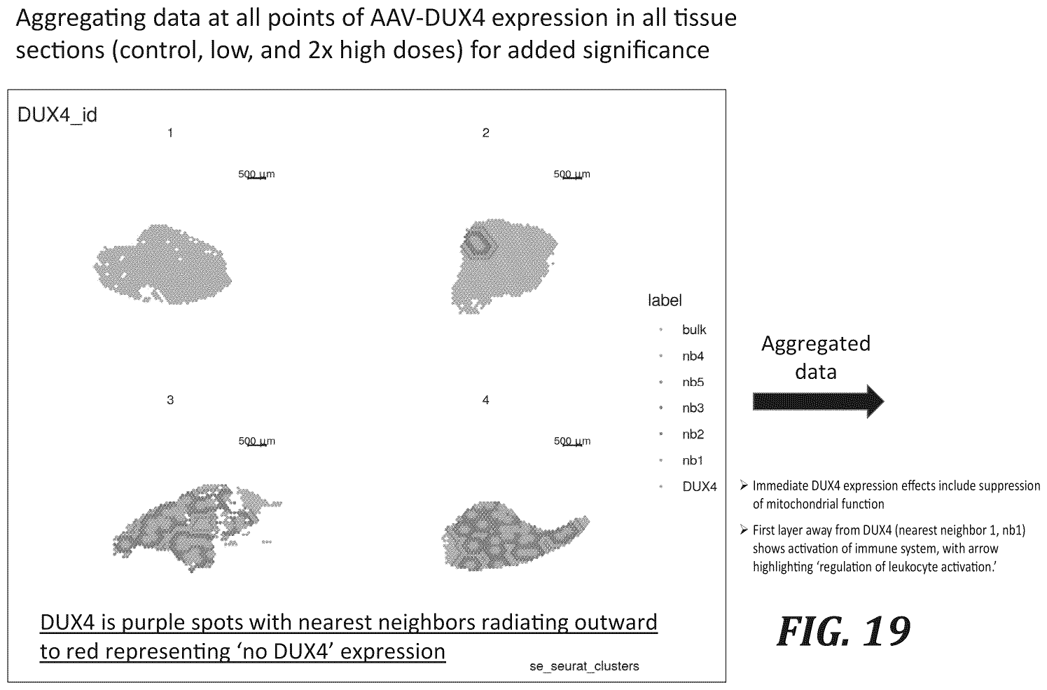

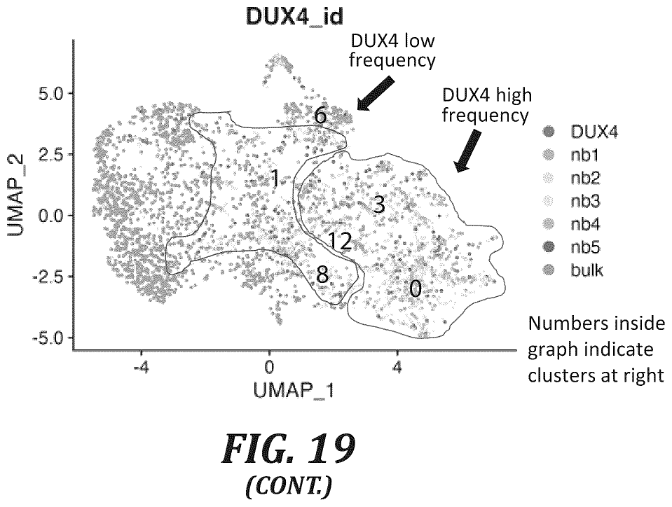

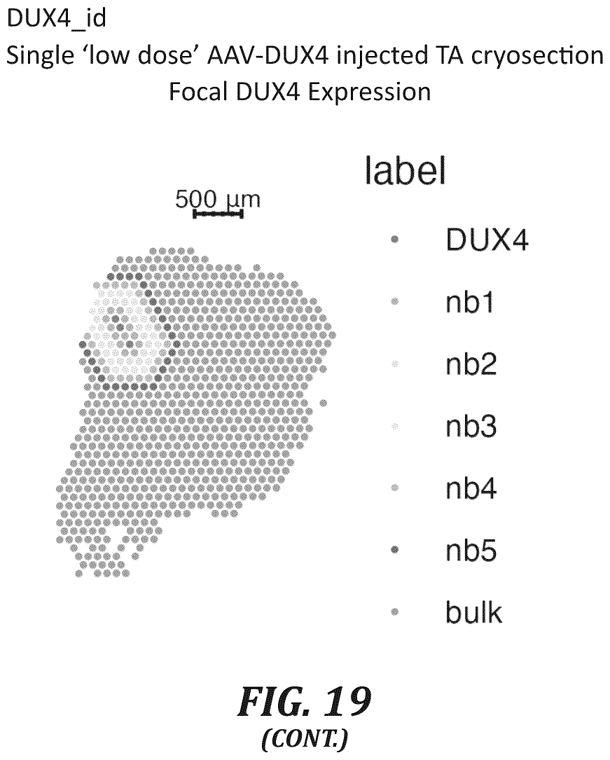

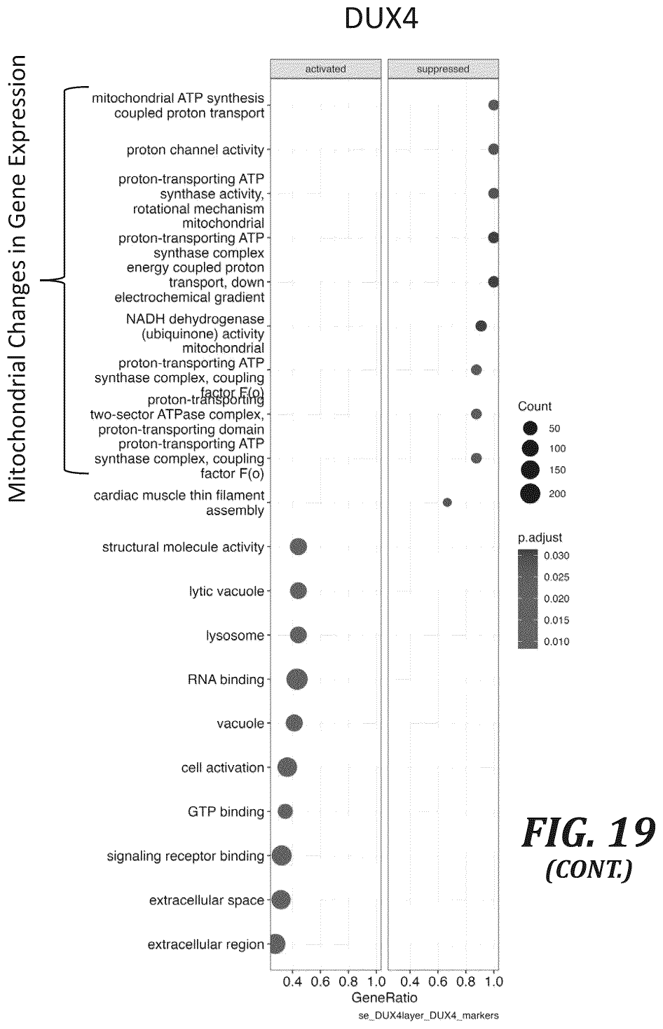

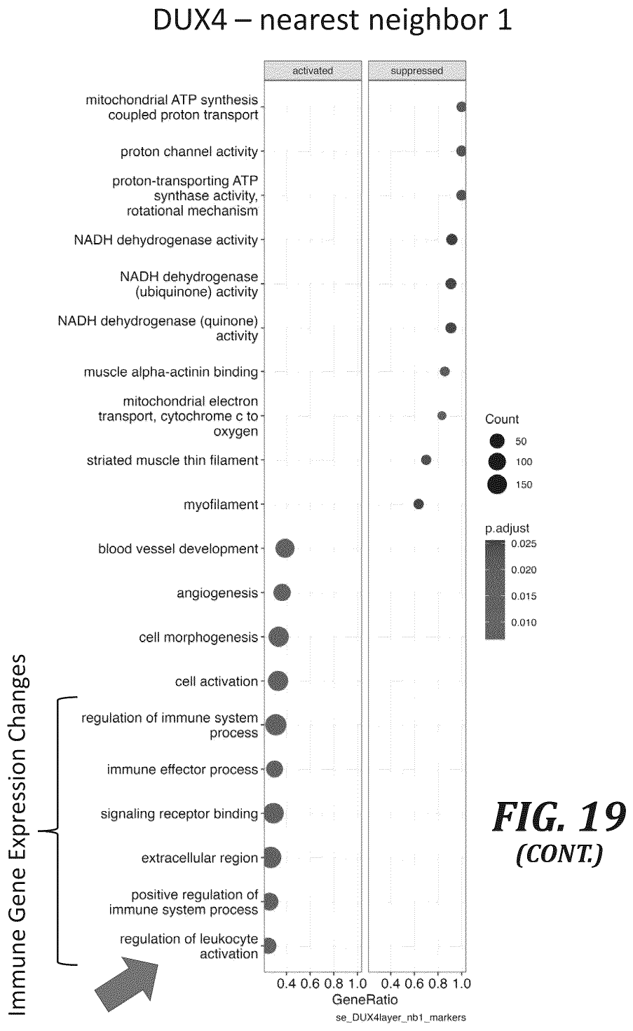

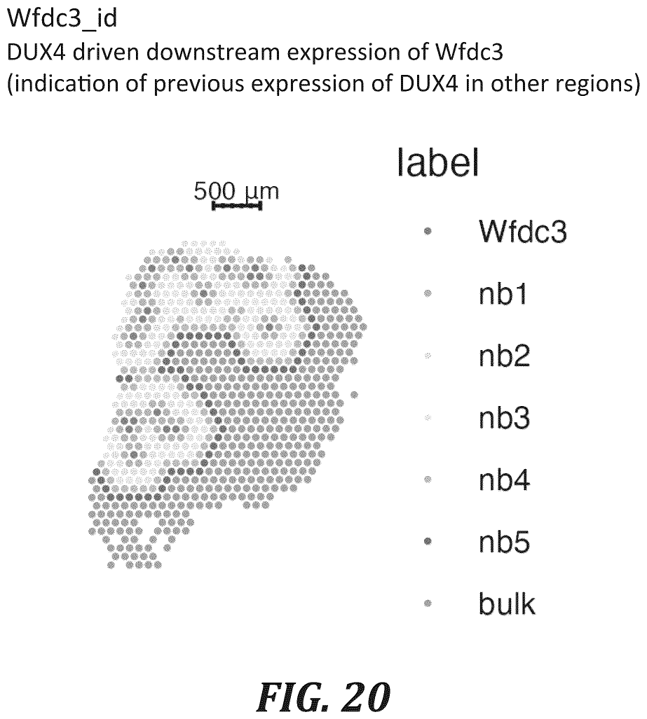

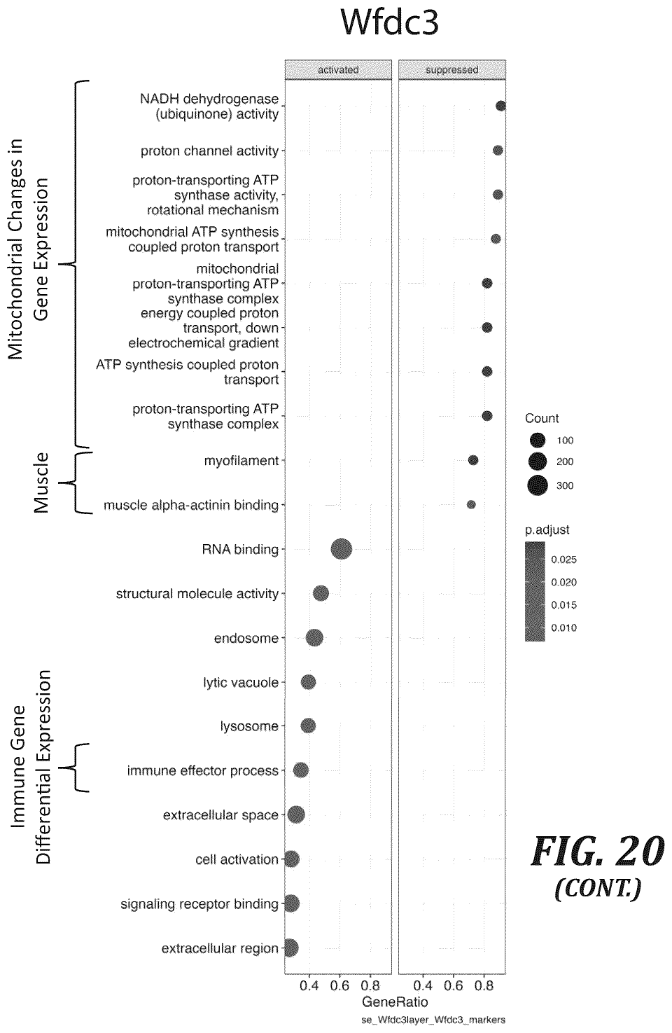

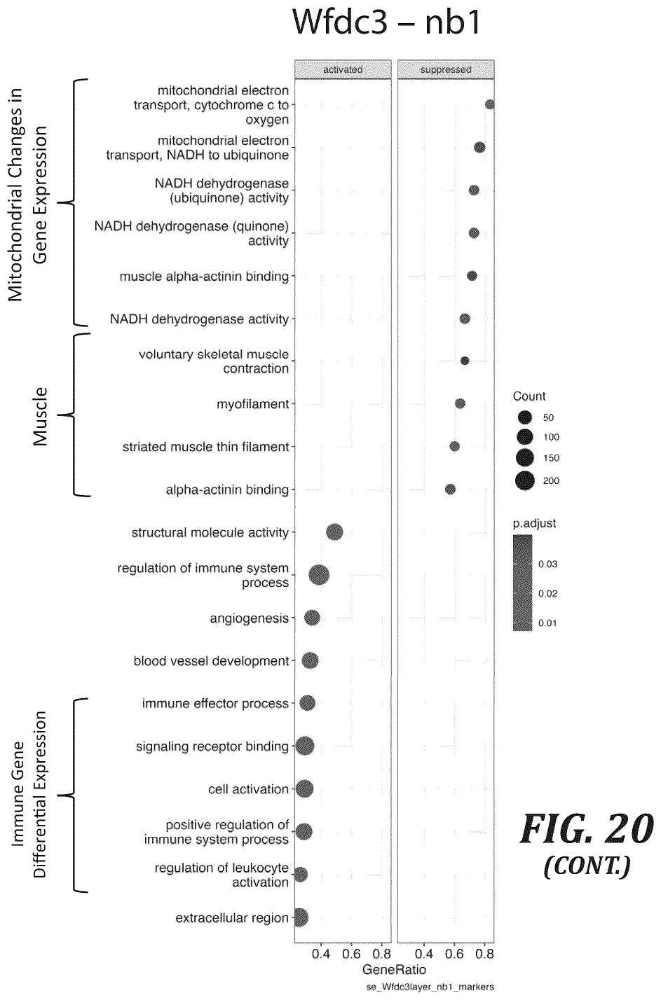

The in situ expression effects of DUX4 expression were also assessed along with the expression changes in proximity to DUX4 in muscle tissue. Using the 10× Genomics Spatial Genomics platform cryosectioned and H&E stained AAV-DUX4 injected muscles were examined for local effects of DUX4 expression on the expression of other cellular genes. RNAseq libraries were made from 50 μm spots across 10 mm muscle transverse cryosections from 1) a non-expressing AAV6 vector (negative control); 2) a low AAV-DUX4 (5×10⁹ vgs); and 3 and 4) 2 high AAV-DUX4 (2×10¹⁰ vgs) injected wild-type TA muscles. Aggregation of the expression profile data from spots containing DUX4 human transcripts showed that transcripts associated with mitochondrial respiration were suppressed, as well as muscle thin filament expression, compared to an increase in immune transcripts categorized in biological pathways (FIG. 18). Upregulation of the immune response in a non-cell autonomous response to very limited DUX4 expression supports early immune cell involvement in FSHD. Moving away from the 50 μm spots of DUX4 expression, referred to as ‘nearest neighbors (nb)’ layers indicates that at DUX4 there is differential expression of in pathways related to mitochondrial respiration and muscle function were suppressed at DUX4 in this analysis (FIG. 19). The lack of immune changes at the site of DUX4 expression is consistent with the finding that DUX4 suppresses the immune response. Although it appears that the immune response is not significantly upregulated in the immediate vicinity, it is triggered at a minimal distance from DUX4 expression in adult muscle in pathways alter in DUX4 nb1. In addition to the ‘bulk’ RNAseq (not spatial) data described previously, where GO biological process genes involved in neutrophil chemotaxis were upregulated, there is ‘regulation of leukocyte activation’ identified in the DUX4 nb1 region analysis (arrow in FIG. 19). Leukocytes comprise the spectrum of white blood cells, including neutrophils. Examination of a mouse biomarker of DUX4 expression, Wfdc3, using the same nearest neighbor approach there are similar pathways suppressed, mitochondrial respiration and muscle actinin binding, while at the site of Wfdc3 expression there is evidence of immune response (FIG. 20). The Wfdc3 protein is implicated in the immune response and is expressed at higher levels than DUX4 in all mouse models of FSHD (although not found in humans), making the Wfdc3 mRNA a well-established downstream mouse marker of DUX4 expression. The larger number of spots positive for the Wfdc3 mRNA implicate previous instances of DUX4 expression driving Wfdc3 expression beyond the detected spots positive for DUX4 mRNA, as DUX4 is known to be expressed in a pulsatile manner.

### Example 6

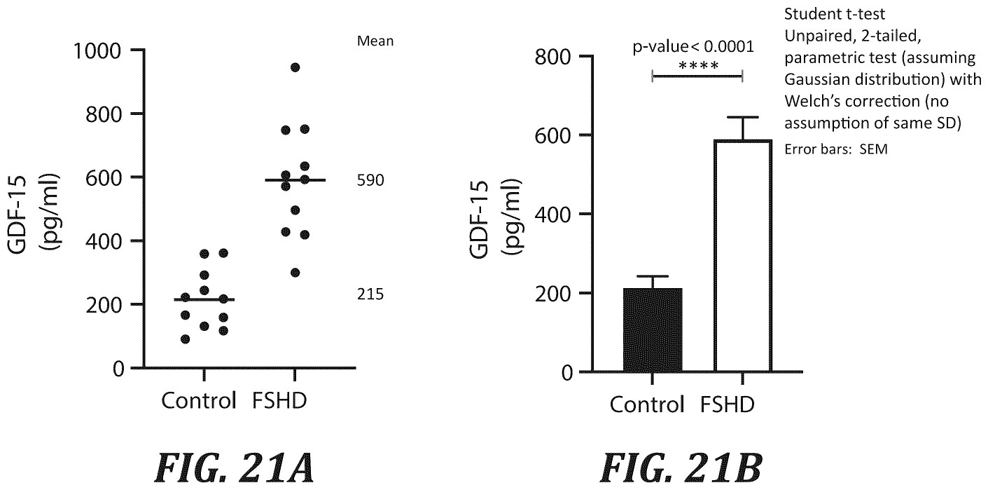

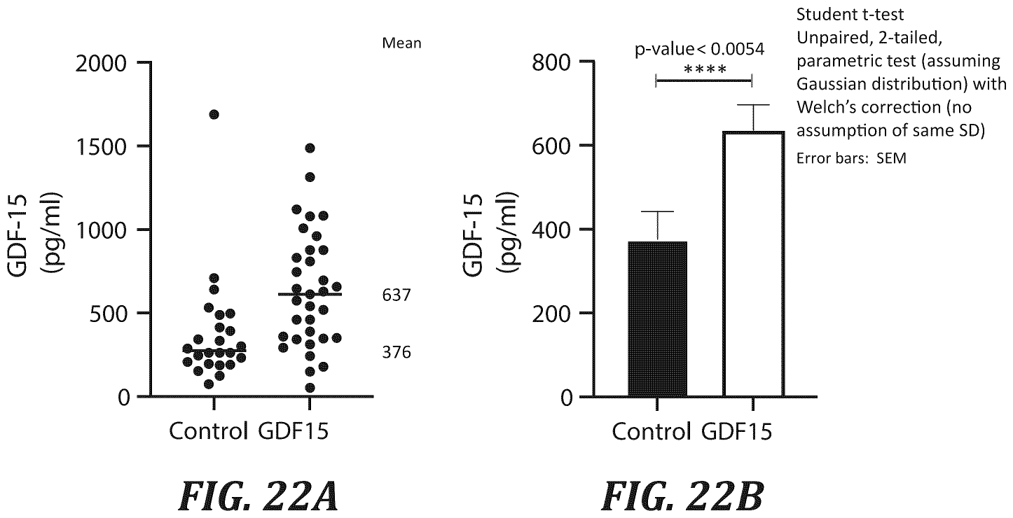

Initial testing with a sample size of 11 FSHD samples and 10 controls did not show a significant separation of the means for fMET, 8-OHdG, or MT-ND6. While they could be elevated in FSHD muscle or other tissues these were not detected in FSHD plasma by the ELISA assay. However, GDF-15 convincingly showed increased concentration in the FSHD plasma (215 pg/ml control vs. 530 pg/ml FSHD; p<0.0001 Student's t test) (FIGS. 21A-21B). The same samples used for the ‘Exploratory Studies’ with the neutrophil candidate biomarkers were then tested for GDF-15 (FIGS. 22A-22B). Data shows that there is still a significant separation in the means, although some FSHD plasma GDF-15 concentrations overlap values for control subjects.

### Example 7

### FSHD Biomarker Testing in Large Patient Sample Cohorts

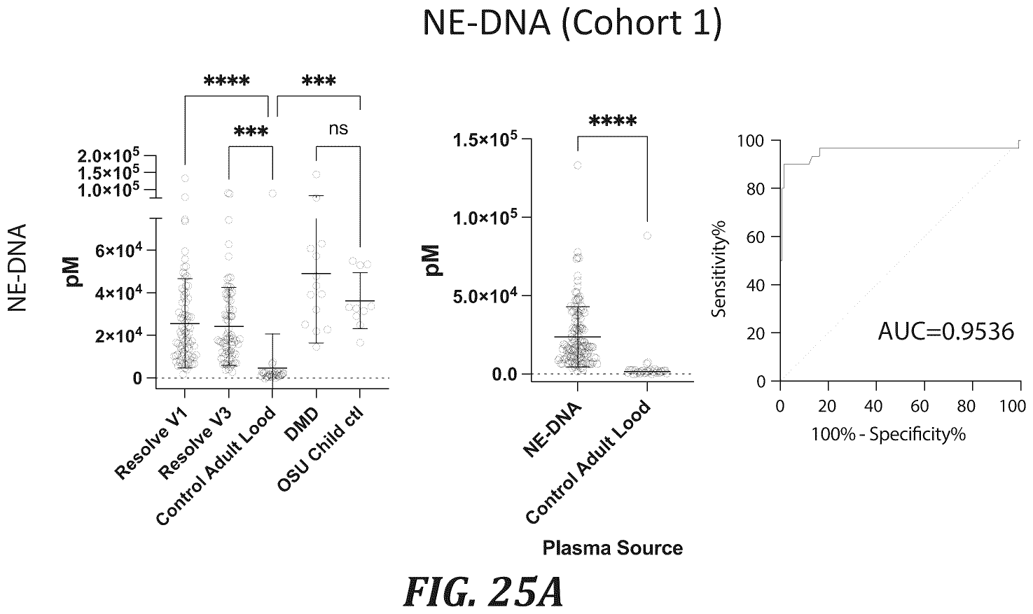

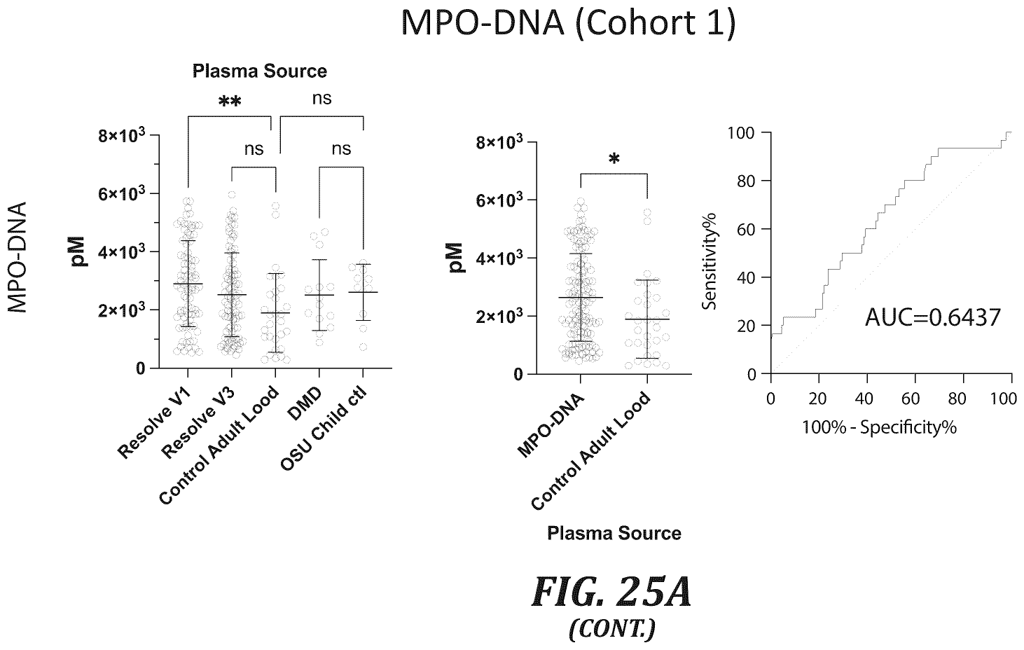

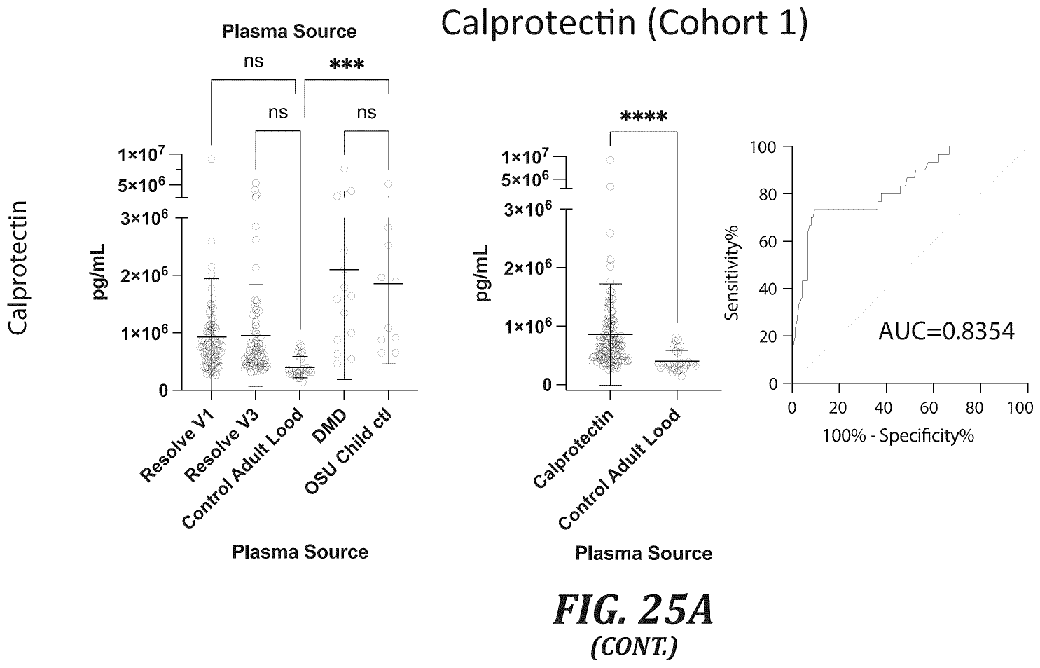

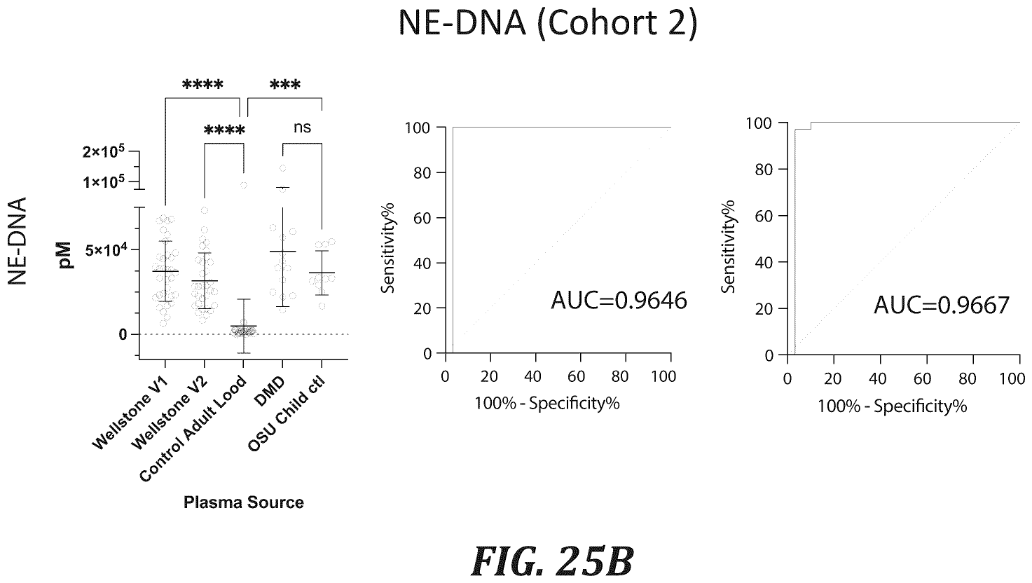

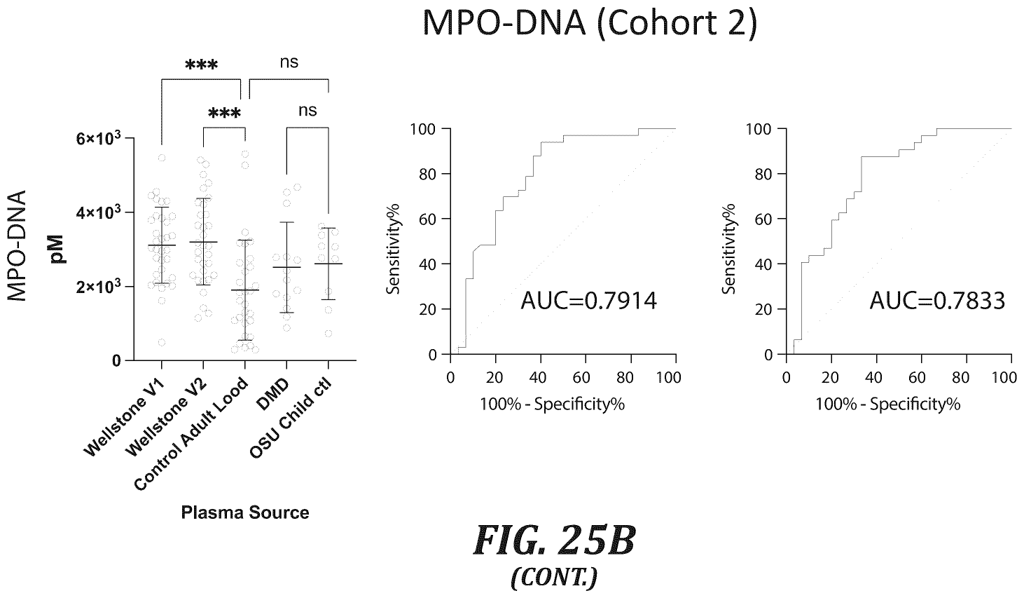

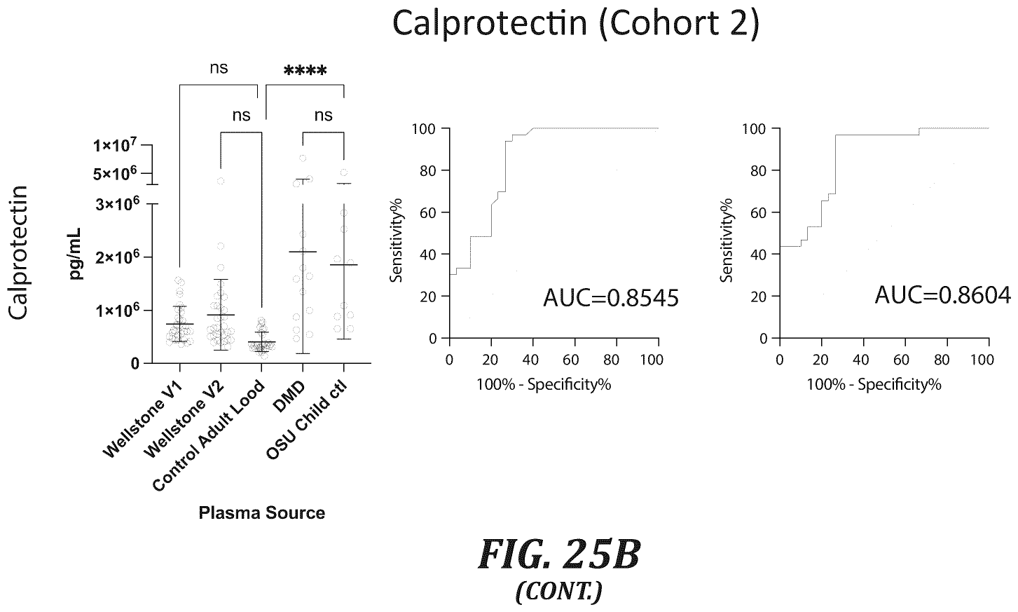

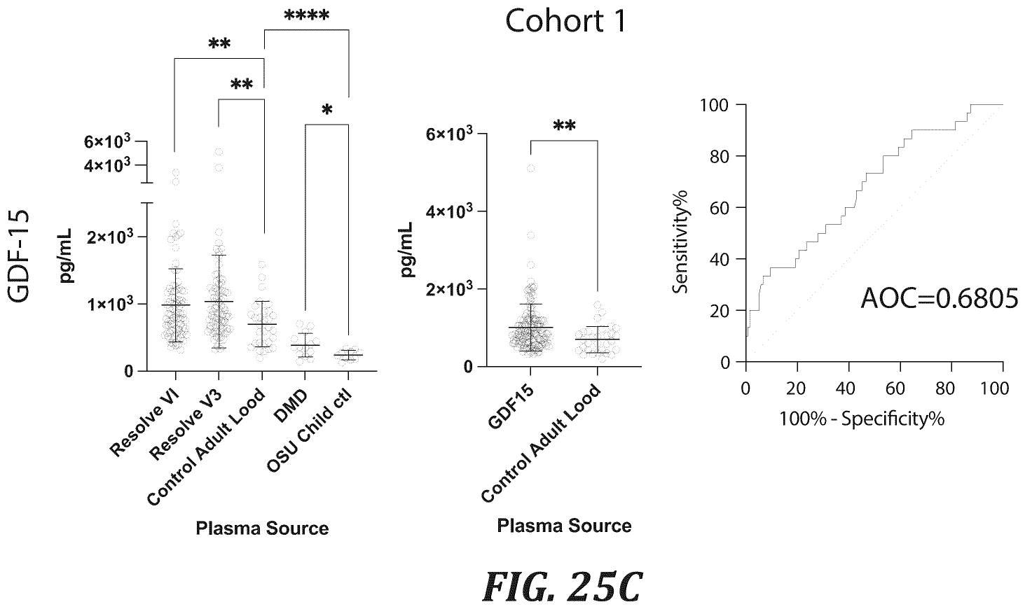

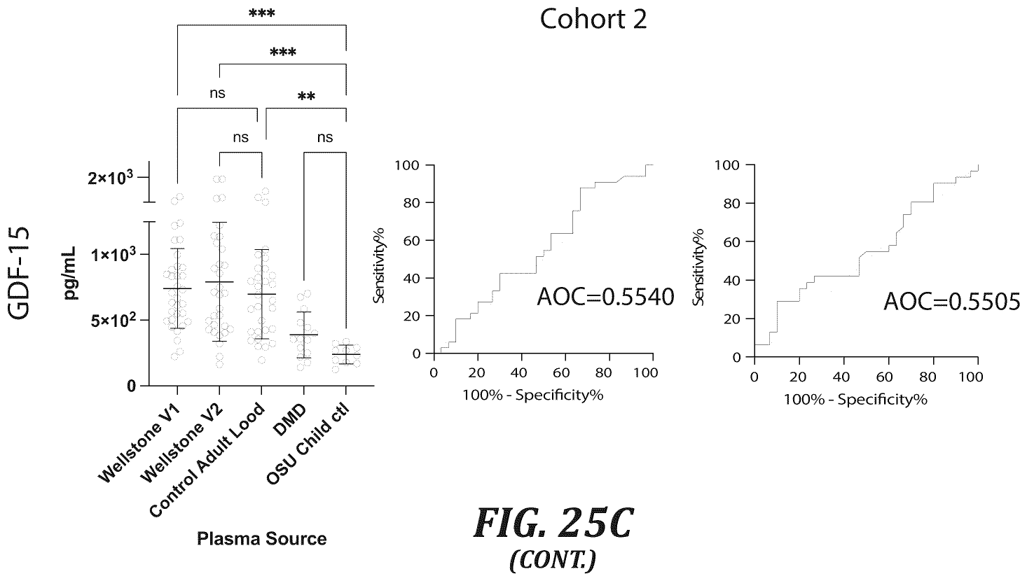

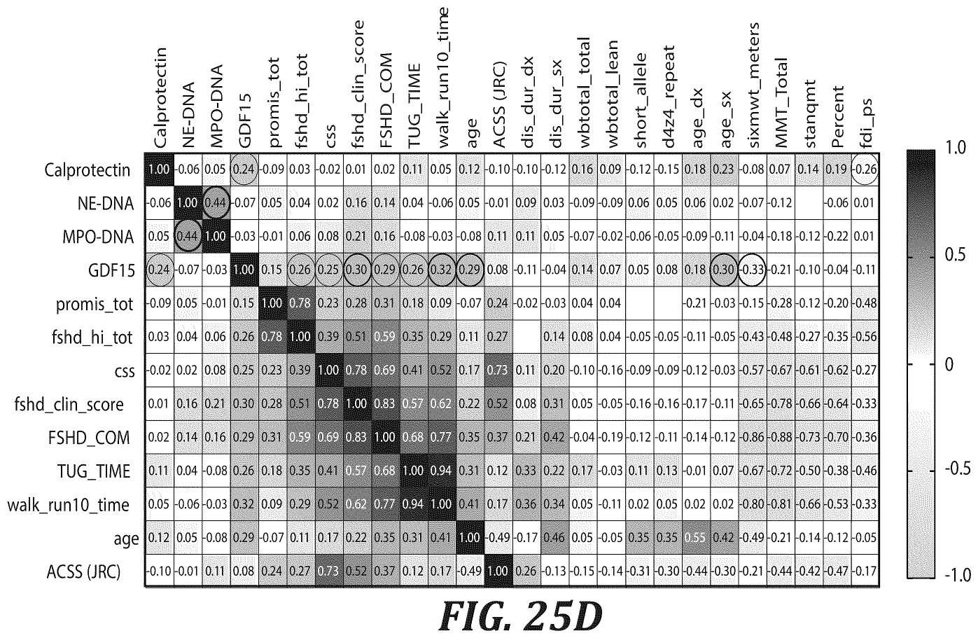

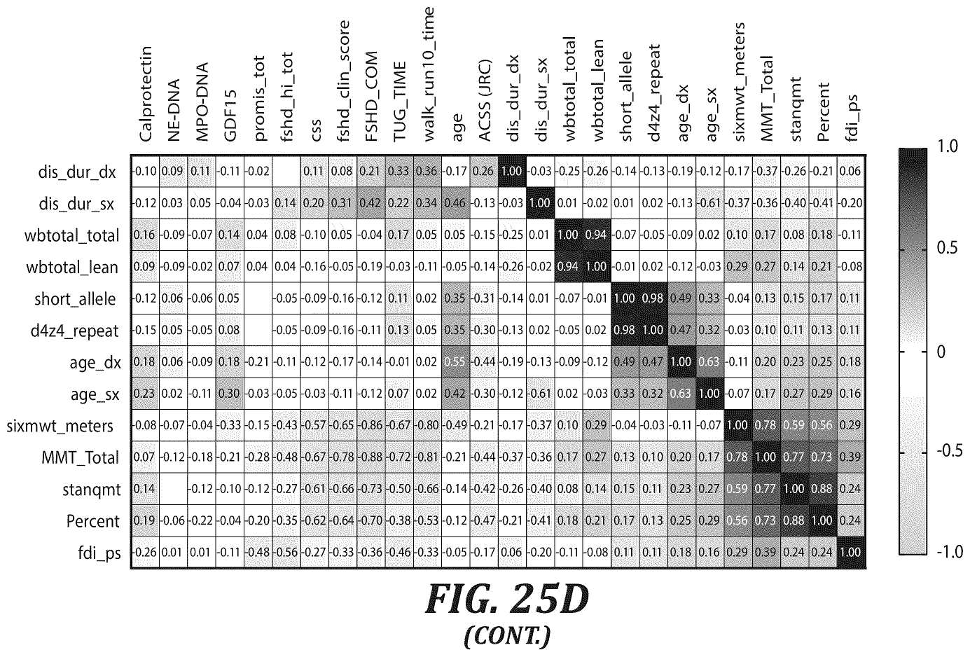

Following pilot studies using small sample sets (11 and 32), a large cohort of samples collected from 2 different clinical studies were obtained. Blood plasma samples from the first large cohort (Cohort 1) of FSHD patients were taken at baseline (time 0; n=84), referred to as visit 1 (V1), or at 12 months (V3; n=78). The second smaller cohort (Cohort 2) consisted of samples from baseline V1 (time 0; n=33) and V2 (time 12 months n=32). Results from the ELISA assays for levels of NE-DNA, MPO-DNA, and calprotectin (calprotectin=direct, single Ab detection by ELISA) for Cohort 1 are shown as open circles (FIG. 25A). The first panel for each part is displayed as concentrations from separate cohorts V1 and V3; the second panel shows the samples pooled (all unique samples) and the final panel depicts the receiver operator characteristic (ROC) curve as an indicator of the estimated accuracy of a diagnostic test using these biomarkers graphed as sensitivity versus specificity. Evaluation of the Area Under the Curve (AUC) (AUC; an AUC between 0.8 and 1.0 is high probability the outcome is true; here meaning that the individual has FSHD). All patient cohorts were compared to results from a commercially available adult control cohort (n=29).

NE-DNA appeared to show the greatest and most significant difference in the means and a very high AUC indicating its strength as a biomarker for FSHD.

FIG. 25 B shows Cohort 2 results from V1 and V2 ELISA assays and ROC curves with AUC noted for each population in comparison to controls depicted by open circles for NE-DNA, MPO-DNA, and Calcprotectin (FIG. 25B). Samples could not be pooled because most were repeat blood draws from the same patient. Plasma from cohort 1 (FIG. 25C) and cohort 2 (FIG. 25D) was used for ELISA quantitation of the myomitokine growth and differentiation factor 15 (GDF-15) which is associated with mitochondrial induced inflammatory responses in muscle (FIG. 25C-25D). Multivariate correlation analysis of demographic, clinical, and molecular disease outcome measures for correlations with levels of the candidate biomarkers is displayed as a heat map. Pearson r coefficients (numerical value displayed in each square) for the GDF-15 protein indicated significant positive correlation with clinical measurements of function (fshd_hi_tot, css, fshd_clin_score, FSHD_COM, TUG-TIME, walk_run1-_time, sixmwt_meters), age, and duration of disease following initiation of symptoms (age_sex) (FIG. 25D). Therefore, GDF-15 is a candidate biomarker for tracking functional decline in the natural history of FSHD. Calprotectin correlates with a measure of facial changes in disease (fdi_ps). Correlations between biomarkers indicate functional relatedness in disease (ie. NE-DNA and MPO-DNA; GDF-15 and Calprotectin). Thickness of circle line defines significance according to p value, ½ point line p<0.05, 1-point line p<0.001, 2-point line p<0.005, and 3-point line p<0.001. The statistical analyses were performed using either a Mann-Whitney U test, a ROC curve analysis, or correlation analysis using Prism software, with the mean with standard deviation indicated as applicable.

GDF-15 concentration levels correlated with FSHD clinical functional outcomes and age indicating its significance as a disease monitoring biomarker.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for diagnosing or detecting Facioscapulohumeral Dystrophy (FSHD) in a subject, the method comprising:
(i) obtaining one or more biological samples from the subject;
(ii) measuring/detecting an overall expression pattern or expression level of at least one neutrophil extracellular traps (NETs)-associated protein in the one or more biological samples obtained from the subject; and
(iii) comparing the overall expression pattern or expression level of the at least one NETs-associated protein from the one or more biological samples of the subject with the overall expression pattern or level of the at least one NETs-associated protein in a reference sample,
wherein detecting an expression or overexpression of neutrophil extracellular traps (NETs), relative to the reference sample is indicative of FSHD.

2. The method of claim 1, wherein the reference sample is a biological sample obtained from a healthy subject, wherein the healthy subject is a subject not suffering from or at risk for FSHD, and wherein the reference sample is a biological sample corresponding to the biological sample obtained from the subject.

3. The method of claim 1, wherein the one or biological sample and/or the reference sample is selected from blood, serum, plasma, synovial fluid, bronchoalveolar lavage, spinal fluid, and saliva.

4. The method of claim 1, wherein detecting an overall expression pattern or expression level of the at least one NETs-associated protein comprises detecting the overall expression pattern or expression level of at least one of Neutrophil Elastase-DNA (NE-DNA), Myeloperoxidase-DNA (MPO-DNA), and calprotectin.

5. The method of claim 1, wherein the method further comprises detecting the expression level of at least one mitochondrial protein and comparing it to an expression level of the at least one mitochondrial protein in the reference sample.

6. The method of claim 5, wherein the at least one mitochondrial protein is selected from at least one of N-formyl methionyl peptides (fMET), growth and differentiation factor 15 (GDF-15), 8-hydroxy-2′-deoxyguanosine (8-OHdG), and MT-ND6 protein.

7. The method of claim 5, wherein detecting an overexpression of the at least one mitochondrial protein relative to the expression of the at least one mitochondrial protein in the reference sample is indicative of FSHD.

8. The method of claim 5, wherein the at least one mitochondrial protein is growth and differentiation factor 15 (GDF-15).

9. The method of claim 5, wherein the method comprises detecting an overall expression pattern or expression level of NE-DNA and GDF-15.

10. The method of claim 5, wherein the method comprises detecting an overall expression pattern or expression level of MPO-DNA and GDF-15.

11. The method of claim 5, wherein the method comprises detecting an overall expression pattern or expression level of NE-DNA, MPO-DNA, and GDF-15.

12. The method of claim 1, wherein the one or more biological samples comprises blood or plasma.

13. A method of treating a subject identified as suffering from, or at risk for developing FSHD, the method comprising administering to the subject an effective amount of at least one therapeutic agent, wherein the subject is determined to be suffering from, or at risk for developing FSHD by a method comprising:
(i) obtaining one or more biological samples from the subject suffering from, or at risk for developing FSHD;
(ii) measuring/detecting an overall expression pattern or expression level of the at least one NETs-associated protein in the one or more biological samples obtained from the subject suffering from, or at risk for developing FSHD; and
(iii) comparing the overall expression pattern or expression level of the at least one NETs-associated protein from the one or more biological samples of the subject suffering from, or at risk for developing FSHD, with the overall expression pattern or level of the at least one NETs-associated protein from a reference sample,
wherein detecting an expression or overexpression of the at least one neutrophil extracellular traps (NETs)-associated protein, relative to the reference sample identifies the subject as suffering from, or at risk for developing FSHD, wherein the reference sample is a biological sample obtained from a healthy subject, wherein the healthy subject is a subject not suffering from or at risk for FSHD, and wherein the reference sample is a biological sample corresponding to the biological sample obtained from the subject.

14. The method of claim 13, wherein the one or biological sample and/or the reference sample is selected from blood, serum, plasma, synovial fluid, bronchoalveolar lavage, spinal fluid, and saliva.

15. The method of claim 13, wherein the at least one NETs-associated protein comprises at least one of Neutrophil Elastase-DNA (NE-DNA), Myeloperoxidase-DNA (MPO-DNA), and calprotectin.

16. The method of claim 13, wherein the method further comprises detecting an expression level of at least one mitochondrial protein and comparing it to an expression level of the at least one mitochondrial protein in the biological sample obtained from a normal subject.

17. The method of claim 13, wherein detecting an overexpression of the at least one mitochondrial protein relative to the expression of the at least one mitochondrial protein in the reference sample is indicative of FSHD.

18. The method of claim 17, wherein the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

19. The method of claim 16, wherein the mitochondrial protein comprises growth and differentiation factor 15 (GDF-15).

20. The method of claim 13, wherein the method comprises detecting an overall expression pattern or expression level of NE-DNA and GDF-15.

21. The method of claim 13, wherein the method comprises detecting an overall expression pattern or expression level of MPO-DNA and GDF-15.

22. The method of claim 13, wherein the method comprises detecting an overall expression pattern or expression level of NE-DNA, MPO-DNA, and GDF-15.

23. The method of claim 13, wherein the one or more biological sample and/or the reference sample comprises blood or plasma.

24. The method of claim 13, wherein the step of administering an effective amount of at least one therapeutic agent comprises administering an effective amount of at least one of DUX4 inhibitor, an anti-inflammatory agent, or a GDF-15 inhibitor, or a combination thereof.

25. The method of claim 24, wherein the DUX4 inhibitor comprises a DUX4 antisense nucleic acid molecule, RNAi targeted to the aberrant DUX4 transcript, or an agent that inhibits the transcription of DUX4.

26. A kit, comprising:
A) a first group of capture affinity reagents that bind to at least one NETs-associated protein at a first epitope, and a first group of detection affinity reagents that bind to the at least one NETs-associated protein at a second epitope; and/or
B) a second group of capture affinity reagents that bind to a first epitope of at least one mitochondrial protein; and a second group of detection affinity reagents that bind to an epitope of the second group of capture affinity reagents.

27. The kit of claim 26, wherein the first group of capture affinity reagents are specific for and bind to the at least one NETs-associated protein at the first epitope, and wherein the at least one NETs-associated protein is selected from NE-DNA, MPO-DNA, and calprotectin.

28. The kit of claim 26, wherein the second group of capture affinity reagents are specific for and bind to the at least one mitochondrial protein at the first epitope, and wherein the at least one mitochondrial protein is selected from N-formyl methionyl peptides (fMET), growth and differentiation factor 15 (GDF-15), 8-hydroxy-2′-deoxyguanosine (8-OHdG), and MT-ND6 protein.

29. The kit of claim 26, wherein the first group of capture affinity reagents are immobilized on a solid substrate.

30. The kit of claim 26, wherein the second group of capture affinity reagents are immobilized on a solid substrate.


SEQUENCE LISTING (11 sequences)

SEQ ID NO: 1 - DNA, 6919 bp, synthetic construct
  source 1..6919: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcgcccaggtgccagcacggagcgcctggcggcggaacgcagaccccaggcccggcgcacaccggggacgttgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcgcggagctcctggcggtcaaaagcatacctctgtctgtctttgcccgcttcctgactagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattggtttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 5941 bp, synthetic construct
  source 1..5941: mol_type = other DNA
ccctgggtaaactgaggcaggcgacacagctgcatgtggccggtatcacagggccctgggtaaactgaggcaggcgacacagctgcatgtggccggtatcacagggccctgggtaaactgaggcaggcgacacagctgcatgtggccgtatcacagggccctgggtaaactgaggcaggtgacacagctgcatgtggccggtatcacggggccctggataaacagaggcaggcgacacagctgcatgtggccggtatcacggggccctgggtaaactgaggcaggcgaggccacccccatcaagtccctcaggtctaggtttggcaggtttggcaaaaacacagcaacgctcggttaaatctgaatttcgggtaagtatatcctgggcctcatttggaagagacttagattaaaaaaaaaacgtcgagaccagcccggccaacacggtgaaaccccgtctctactaaaaatacaaaaaattagccaggcgcagtggctcacgcctgtgatcccagcactctgggaggctgaggcaggcggatcacccgaggtcagatgttcaagaccagcctggccgacagggcgaaacactgtctctactacaaatacaaaaattagccgggagtggtggcaggtgcctgtaatctcagctattcaggaggctgaggcaggagaatcacttgaacctgggaggcggaggttgccgtgagccgggatcacgccaccgcactccagcctgggcgatagagcaagactctgtctccaaaaaaataaattaaaaaacccacattgattatctgacatttgaatgcgattgtgcatcctgaattttgtctggaggccccacccgagccaatccagcgtcttgtcccccttctcccccttttcatcaacgccctgtgccaggggagaggaagtggagggcgctggccggccgtggggcaatgcaacggcctcccagcacagggctataagaggagccgggcgggcacggaggggcagagaccccggagccccagccccaccatgaccctcggccgccgactcgcgtgtcttttcctcgcctgtgtcctgccggccttgctgctggggggtgagtttttgagtccaacctcccgctgctccctctgtcccgggttctgttcccacctctccatagagggccccaccagtgtgggtccctcatcctcacaggggaggtgccagctgggacaaggagaccagaagagactgaggttctgagcggtgaagccaccaccaggagcccagagttggggtttgaaaaccggggagggggggggggtcgcaggtcgccctctgggttcaagtccaggtctgtctgtgccttggaggggcaccgtggggaggtccctttgcctctccgtgcctcagtttcctcatctgaacaacaggggtgcgaacggccccgatcccgtgggttcccggtgggggatcccgtgggttcccggtgggggatcccgtgggttcctggtgggggatccagaggccccgtggccgggaggggacaggctccttggcaggcactcagcacccgcacccggtgtgtccccaggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtgtgagtagccgggagtgtgcgcgcccggctcggaccccgcgtcccggtctgtgaggtgggtggggggaggccggggccggggctgctggcgggggggggtccgtccagggcccgcggggcccctcgagcaccttcgccctcaggcccgtcgccggatggggacgacaaggcgcggctgagccccgacccccggggccgcccctgagccccgcctctccctccccggcagaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccaggtgccgccgggcggggcggggggcgcaggggcggaggccagaggcctggggagggtggaggctgcgacggaggggcgcgtcggggccgctcgtggggacctggggtggcatcgtgggctgggtggtcccctctccgcgcctcggtctgcacctctgtgaaacgggaaaatacccgccatgggccgttgaggggttaaatgagatcctgcagggaggccccgatctgctgtcaatcaacaaacttactgagaagggaggccccgatctgttgtcaatcaacaaacttactgagaagggaggccccgatctgttgtcaatcaacaaacttactgagaagggaggccccgatctgctgtcaatcaacaaacttactgagaagggaggccccgatctgttgtcaatcaacaaacttactgagaagggaggccccgatctgctgtcaatcaacaaacttactgagaagggaggccccgatctgttgtcaatcaacaaacttactgagaagggaggccccgatctgctgtcaatcaacaaacttactgagaagggaggccccgatctgttgtcaatcaacaaacttactgagaagggaggccccgatctgctgtcaatcaacaaacttactgagattctttgtgtctctccattcaccagtcctgtggcccagggcaggggccgcctctgtctttgggaaaaggggcaaaagtccccacctttccacccctgtccgcggcttgcagttctggttatttcctgggcgccgggccccgtggctcaggcctgtcatcccagcactttgggaggctgaggcgggtggatcacgaggtcaggtgttcgagaccagcctgagcaacatagtgaaaccccgtctctactaaaatacaaaaaaaaaaaattagccgagtgtggttgtgggtgcctgtaatgccaactactcaggaggctgaggaaggagaatcgcttgaaccccggaggcggagattgcagtgagctgagatcacaccactgcactccagcctgggtctcaaaaaaaaaaaaaaagattcctccctgggaagggttagagggagagtttccttgtcactaagttttctcatagctctcacccagtgcagtggcgcgatcgcagctcactacagcctccatctcctgggctcaagccaccctctcagcttggaatggggggtagctggaaccacaggtgccaccacgtgggtccaccacgtctggctaatatatatatatacacacacacatacatatattataaataataaatatatattttatttaaataaaatatataatatttataattattttataattataataatatttatataattataaatatcatttataattataatatttattattttataaaataataaatataaaatatataaaaatatttttataaaataataaaaatatatatatacacacatatatatatattttttgagacaagtctcgctctgtcgcccaggctggagcgcagtggcacaatctcagctcactgcaacctccgcctcccaggttcaagcgattctcctgcctcagcctcccaggtagctgggactacaggcgcccgccaccacgcctggctaatttttggtattgttagtagagacggggtttaaccatgttagccaggatggtcttgatctcctgaccttttgattggcccacctcagcctcccaaaatgctgggattataggcgtgagccaccgcacctggcaatttttttttattatttttgtagacatggggctttgccacattgcccaggctggtcttgaatgcctggcctcaagtgatcctcctgcctcgccctcccaaagtgctgggcttacaagcatgagccaccgcgcccggctgtagtttttttgttaactgagcacctactgcttcctgcactcaagccacatccagggacaacctccaacgccctgagccttggtgacggctcccactctacagatggggaaaccgaggcttgccttggggagcagagtgtggggtgggtatcctgccctgcaggatcccagaaccacagtggaacctgagatggggaaactgaggcccggagaggggagggtcatcatcactgccccgtgtgacgcgctgacgatctgtccccaccgccacagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcgtacgtgccctgggtgtccctctgctccccacccgctcccagcccggactgcagcaacaggcaccgtggctagaccctaggagggacttcccaaccctgacaggcggcgggcaggtgggcagggcctcgcagtccagcttccccaccttgtctgcctccacagggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaatgtgtttgatgctccttggctgtgtgattgggtgttgaaaatggtcagtaggtcgggcgtggtggctcacacctgtaatcccagcactttgggaggttgaggcaggcggatcacttgagctcaggagttcaagaccagcctgggcaacatggcaaaacccccgctccattaaaaaaaaataaataaataaaataaaataaaataaaatggtccaagcctggcccagtggcttacgcctgcaatcccagcactttgggaggccgaggctggcagatcacctgaggtcgggagttcaagactagtctgccaacgtggtgaaaccccatctctactataaatacaaaaaaaattagcttggcgtcgtggcgcgtgggaggctgaggcaggagaatcgcttgaacctgcgaggcgaaggctgtagtgagccgagattacgccactgcactccagcctgggcaacacagagagactccgtctcaaaaaaaaaaaaaaaaaaaaaaaataccggtgcggtggctcgtgcctgtaatcccagctctttgggaggcccaggcaggcggatcatctgaggtctaaagttcgagaccagcctgaccaacatggagaaaccccatatctactaaaaatacaaaattagccgggcgtggtggtgcatgcctgtaatcccagctactcgggaagcctgaggcaggagaatcacctgaattctggaaggtggaggttgcagtgagccaagatcgctccattgcactccagcctgggcaacaagagcgaaactccatctcaaaaataaataaataaataaataaataaataaataaataaataaataaatataaataataatgataataataataataaacaacaaactagaatccagagtctcttgggcatctctggagtaagccaccaccagggggcagcacagccagggcaatggagcagaagtgacttccccacattgtgtttttttcttttttgtttttagatggag

SEQ ID NO: 3 - DNA, 13081 bp, synthetic construct
  source 1..13081: mol_type = other DNA
gtttggagacagccctctaccagaagccaagaagaaaggggagtgaggagggatagaggaagttatcttggtcctgcgcccacagtccccagggtcctccttcctgtgaagcccaacggtctccagccagatttcctgtccccttagccccaccaagaaccagaggctgcccattgggtggctgttggatgcaggttactgttggagtgggggatggccacctgaggccaattgggtcatctttactccaggtctccctttcatcctcctgtcttccctgggggcactctattccctgcaattccttgggctaccagttcctgacttttgttcctttcaaaggaaccctggataaccagtgtaaccagaatttcagaggggttagttgtgtgtatcccctggggacaagcactggtataggcacacaatggtgagctgagaaatcttgggctggtagtgctaaattcaaaggctggggacaggctggggccagtggctcatgcctgtaatcccagcactttgggaggctgaggcgggtggatcacttgaggtcaggagttcaagaccagcctggccaacatggtgaaaccctgtatctactaaaaatacaaaaattagcttcttgcctaaggaaaaatacaaaaattacttcttgcctaaggaaataattgatgatgtggctagaggctagggcgtggtggcgggcacctgtaatcccagctactcgggaggctgaggcagaagaatcgcttgaaccattgcacatcagcctgggcaacagagcaagataacgtctcaaaaaaaaaaaaaaaaaaggctggggacaatgctggcccctcctttcctgccccttccccccatttcaggggcccctctgtgtgtaccttccaaccccagtgggggaggagaaagagttcagtctccaggatcagaccttcctctacctcaccccacccccagcttagaggacataaaagcgcagattgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccaggtaacagttcccaaggtgggagaagatggtgtgttggggtggttgtgttccagagaccccttttctcagagcaggccttcctagctctggggcctgatagggggttggggcccattcctgactttgtgatccctgctctgggcagctgtcctgggggaggtggacacctcgttggtgctgagctccatggaggaggccaagcagctggtggacaaggcctacaaggagcggcgggaaaggtggggcacgaggcactgcccagctctgggcaaggatgtcccaggcctttcagagaagcagcaggcagcagggagcttgaaggtgggagagggggactatggggacccatgtggactccctttctgtctgtctgtccctgtgtttctggatgggagaacagcttaaaagccatccttcccaaagccttgcctctgtctggacacccaggtccatatcctttttataaaaggaagccttcctcccacccgtgggcctgcctgcatggtcctgcatgtctgcccctgggctggggctctgctgggtgggtctgtatcccttctctggccctcactcctcctttctccaagcagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactggtactgttgcccatcacaccccaggtccctgctccactagtgactcctcctagggaccccagccgtctctcagtgatcccctcaacttcttcctccagggagtctcaggagtctccagggctcctcagcctcaggttgcctgggataggaagtgaggcggctcagctcccccattgttctttcccccggcagatgtgctgacgcccgcccagctgaatgtgttgtccaagtcaagcggctgcgcctaccaggacgtgggggtgacttgcccggagcaggacaaataccgcaccatcaccgggatgtgcaacaacaggtgcggctggctgggggtggctgcaggaaccgggctcagagaggcgtcccggacgccacaagcctcccggtgtcagcgccctgtcctcccctgcagacgcagccccacgctgggggcctccaaccgtgcctttgtgcgctggctgccggcggagtatgaggacggcttctctcttccctacggctggacgcccggggtcaagcgcaacggcttcccggtggctctggtgagcgccggcgggcagagggggcgaggcccggccacgcggtgcgcggacccaggcgccagctgatctccgtgtcccgcaggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaaggtggccctgcttcccgcccactgcctgggttgggagaggggagattgtttctggaaggggccatctcccttctgtgcccaggtttcctttcccagacgctgaggaaggctggccctgcctcccttgtgtccacagcactggctgctcagctatgacctgtctccttcctgcgcctgggctcagccagctggcagccagggccgtccatttgctcactgcctctggcaggccaagggagagagggttgttcctccttggctgacaggagtcctgttggtggaatcactggctctttgttaggttgggagagggttctgggaggagaccttctcagtgccccccaagcctccatgctcagtcctgtttccgccccactgagagggcagccccaacccccatcacacagaaagagagagcctgggggtctggtgggtgccgtgggacagggctgggagtgggcagggtggtcgaggcactgctgagtagggaaggaggaggagagaaaggagagagcgcctgagagagggagagacagacaggagaatgtcagggccagagggagagcaggcacagcagagagaaggggagagagacgggcgacgctttagtgaggagggatccgaggcctggcggtgcccagacccaggggttgcccaggttcccagttcagtgttctgctcattaaccctgcacctcagaggctgttgctgatggcgccctaggcagcgatccctttcgggcctccggaggcctctctgcaggttgagggtaccagaggtcctgagggcagggggagatccagttctgcctgggcaccttccttgccctcggtgagccagtctagcctctctctgtgcctcagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggtcagctttggggtggggaccagaggtggcataggagatgttccctgttggagccacagtgagtctgtttgtgagcagcttgtgggtttgtacttagagagactgtcctcaccagccattactatcaacttcatgatattaatccagttgccttggtaacaagtcagatggagccagaaaagcagaaaagcaaacaacctctccaaccctaagatggagacccaggcatggctgaggagcctctgtccatcagtccattgctttctctgtcccttttttcccttctggactctggaagacaagagagtcaaatccctcaggggcagggaactaaaggacagagaacgctgtggtgccctcaccctctagagcgacactatccaatgtggtagccactagctgcatgtggctattttaattttagttattttaaattaaataaacccagtttctcagcggtgctaaccacatttcaaatgctcaattgccttacagtctagtggctccccctattgggtggcacagatgtaaaatattttcatcatctcggaaggttgagttggaccacactgctccaaaatatcccctcctgcgcatcttaattacccataataagagatgatgggattttaaatgtaaacaattggcagttttggagacacaattggggcagtgatggcagccatgaccagacattttccagtgttttttttttttctaagacggaatctcactctgtcacccaggcttgagtacagtggcacagtctcggctcactgcaacctctgccaactgggtttaagtgattcttctgcctcagcctcctgagtagctgggactacaggcgcctgccaccacacttggctaattttttgtatttttagtagagatggtgttttaccatgttggccaggctggtctcaaactcctgacctcatgatctgcccaccttaacctcccaaagtgctgggattacaggtgtgagccaccgcacgcagccattttccagtgtttttaaatgtaaacccaatagtggacctcaggccctttcctctggcctccctctgcctcctcctgatggttcctgctggtctctccctggctgggaccctgggaggaggatccaggtcggactttctgcaagagatgcccgacctcttctgggctagttaggcaagggcagccctccttagccagactcagctgcccacagcctggctttgatgagccccatgtcccaagcctgggtcattatgacctctagctgttcatggatggagatgctgctctcagagaatttcctttccaccagcttctagagctggcaaggactgaccctagtgtcctgaccctagccctctggcccctgcagagaagggttctgggaggactaggggaaccaggggggatctgcttagttttcttgaacagtctcaggtgtggactccacatatctcatgtcctagccctctcccctggggcaggctgcaccattggcatgtagtaaagggcagggctgacaactctgttcaaatggtagtggctttctggagtcctgtgttgagaaggattctgaggttgcatccaattttagcaggaaagagtgctgtgatcgttagctgcatcagacatgggcatggatgtaggctgtgccaacacgcatgcttcttcccagacactccttggaatcaagagaatcaatgaaccaagcagtgttcataagtctcatactttgtaccaggcactctgttaagccccatgtatccatatgtgtgctaggagggagggtaaaatataaaaaggcacaagccttggtcctgtgctcaaggagctcccagtttcaggtacaggaaagtggcatgtcatgtgttggaagtgatacctattgaatgagcatccccaaccagcagcggccctgagctgaggcagaagctatcattgtggactggggcatcagggaaggcctcctagaggagtgtaaattggggatagtttgaaaagatggatggggaggaggggtgagtaggaggcatttcaggccttggctggggagggggtttcagtggagcaaatcttttctgggatggaggccttaagaatgacagtggcttttgcctccccaaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccaggtaggccgtcttgaacaccgggcacacgggatccagatgtgtccctgcaacatcctagctgtgtgaccttgggcaagttcctaacgctcctgagccttgccttcttcatccatgaggctgtaaggattataatacatatatgaaaacacctttcagatagtaagtgcttgacaaagcctccttcccttccctctccttcttcgcaggtttgctccctgcagtcttctttcctgtgggtctgttattggtgttgggagggttgaatgtttggtttagcgcccaagtagctgttcagtaaattcttcttccctccatctcactgtcactcttagctcctttacccactcacctcttctttttaaaattaaaaaaattatagggctgggcgcggtggctcatgcctgtaatcccagcactttgggaggcggaggcgggtggatcacgaggtcaggagatcaagaccatgctggctaacacggtgaaaccccatctctactaaaaatacaaaaattagccgggcgtggtggcgggcacctgtagtcccagctacttgggaggctgaggcaggagaatggcgtgaacctgggaggcagagcttgcagtgagctgagatggcgctgctgcactccagcctgggtgacagagcgagactccatctcaaaataaataaataaataaaataaaatttaaaaattattattattattttatttttattttttgatacagggtcttaccctgttgcccaggctggagtgcagtggtgcgatcttggctcactgcaacctccacctttcaggctcaagccattctcccatctcagccttctgagtagctgggatttcaagcacgggccaccacgcccggctaatttttgtattttttgtagagacggggtttcgccacgttacccagtgtggtctcaaactcctgggctcaagtaatccgccctcctcgacctcccaaatccctgggattacaggcacgagtcactgtgttcagcccactcacctcttctttttggctctgtcataatccacccaatgtccttagcctcatttacccctggggcaatgttcctggttctgtctctcaacatctggtgcttcattgcccccaccctctccctcagaggcagaggcagacatacacacactcacacacacacacacacacacacacacacacacacactcctagattctggaggagagagttttcggtgctaagggtgagtttttgatttgcactaagaactcaagtgaaattcatttacactccaaaataacttctagactctagaatgtagagtgagactattccatgtcttcacttttcacagggaaaatcatgccagaatggcccttcgttccttcctgcctccctgctttccacaaacctaaactgagccttactgtgggcaggcacttggtgggcactgtgaagaatttcaacaacttcaatttcccttgctctcaaggactttataagatagttgcagaaatgagacattaataacttgaggacaaggaagttatcacaatgactaaaaaagtgatttgctatttagtcctctaaggcagagggcaatataattatccttaatgtacatttgaggaactcaggctcagagaggttaataattggtccaaggtcacccagccagcaagcagtagaactctatttatgcctctcagtgccactctgttaggcccaggtccggtattgtgggaggctattccctgaccatagagggatggaaggggtccccgaagccaagagcaggcagagactctggccctcctgtgctgccaggcggcatttgctgtggaggagtgggtggagaggccattccaaatgacttgtctttagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtaagcaggacctaggccaggagcgagtgggctgacagctaaaggtggggtagggatcaccttggctctagggagctagggtggggagcagtctgctttcctcccatcttggagatctggtctgactctctagcctcagttcctctgactctgcttccatatctgataataaacagtggggtggggatggccctcagagggaactggctacttcctatgtgttgtggctcgctgtgacccaggaagccagggcctgggagcatcccaatcttgcaagtctgaatggcataatctcctgagacctatatgtccctggggtcatgtagtgaccggtgaggacctgctcactggggccaccaccttggcaccaggcactgcttgtgcccagccctcttctcgaatcctcctgaccctacctggacttgtccctgactccaatctgagctctgatactgagccagatacttcccctgacctggctcctctggtccccaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggtgaggggctgcaggagtctcccctgatgctcacctcccctgagctgcctcctaggtagcccatcaccccccttcccactctagggtccctgccctctccaaagcatattagaggcactttgctctcaggaagtagcctgaggccaggcccctgccttttcccggacccacctgataaatttccagctcctctcaacctcctttgcatgctgtaattctcatattaggggcatcgtcatcataatgataacccacagtctgaaaatctgtctctgtcatcattccattttgcttcattgtacgttaactctgggggaagagattattattgccttcattttactgatgaggaaactgaggcttgatgagattcagaacctggccaggttctctagccaagtcagtggcaaagctgaaatttgaagccagattagcctaacctcaaagcctgtgcctatgttatggggtagcccatgtggctgaggcttctagacacatttcacctctcatgggtcagagacagacacatcgggctgctctggagggggacaaggagatgtccctattcttggacccagcaggaactgtggcagtttagggtttgccttatggtaggagggcaagagaaagacagagagggtcccagagaggcccagttgtgccccttggtctaagccaaaggtacagggttccttgaggtgaggtccttccctccctctatgaccttggttgtgttgggaaggtgggttttctgggctgctggcgatatgtgaatcttccctgctgtctccagtgacctccccaccttgaagcagagagacctgcccatagccacctgtcccctcccccatgcaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggtgaggaggggcaggcgtcgtgggccgctgggtggctgtgggcccatccttgactctcttggagcccaaatttcctcctgtcagttgaaggactggagggagtcagtaattttccagtgtgttccaggaatcctccagatgccctggagtgcccctaatgtgccctgaacctgttgggggtgcagcaaggaaagacctggacccctggttgtaaagggtggaagatagaaaagtgccacccttagctacttcttcccatgggtttcactgtgccaccatgtgggtgtcagtcaccagtgggcagtcccctcccagctttcccacagctccccttagtgtcatttcctcgtggtcctgatgtcttcatgtctagttcttgcttagcctgtactgcacctttgggaaaattagaaaaaggtgcccctctctgggtgggcgaattataaaccacccccaagattctcttccactcctgtggatgcagctcctggccagggcacagggcttcaggagtgtgtgtgggcccgggtccccctccagcatggtgccctggaggccgccgggctaggggtaagggaaaggccactgggcagctgtgctttactctgcacgatgagtggagcatcacttgtgtgaaagcccctgggctgcccaagggcctggggccctcctgtgcttccaggtggcatttgttgtggctttgttatatcctgggagcagcacaagcccatcgatgccctgccagcccagaatatccttgggcacagtgtccatgggtgttccccatgcaggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctccactctgtctgtgtctgaggaggaactggcgcttttgcagaggcttactctgggctctgagcttgaaccagtgtggtctgtgctcagggccctgtgtttctgaaccgagggtgaggatgttggggagttttcctttgagatccgtacgtggagggcagccatctcttggaagcctgggggccggggcatgcggtgcagaccctcctccctcaccttccacccctgccaccctgcagggaactgtggtcaccaagccctcatgtttgttcattgctctagcaaacgtttgctatgtgttcactctgtgtcaggcctgtgccaggggctaggaagtggaggggacaaaatgctgtgtggtaggagagaaagtacagagacctggtgaggaggcaggcccttccccaacaagaggggcctctgcatcctgcatcccagcagcctggcacaggggttcagaggccctcaagacagtggactccttctcattcatgttgcacatggctcctggagaagggagagcagatggctgggcccagaggacagggccagcactgagggaggggaaggagtgtatttccctagtagtcttgcctggagcttgaaccctgcgtctgactggtggactccctccctctcctcttccttctcaaatatggggagggggctggaagtggtgtaggacggtggggtctaatgtactcatggggcccctggtaacgttaggacctcaccatggtaagaagcagcaaaaatactgcttgtcctcatttctgtgatgaggagagggctgctcagaggggtgaagcgactcgtctgaggccttaaagaagtgggtggcagtgccaggctgcagccgaggtcatctggctcctgggctcttcccacaggatgactgggtgtctcccactcccaccccaccagttgggggatctggaggccctggttgggctaatctttcccagggggtcattttaaaagaagaacatatgaatatgtattggg

SEQ ID NO: 4 - DNA, 5019 bp, synthetic construct
  source 1..5019: mol_type = other DNA
tagagacagggtttctccatgttggtcaggctagtctagaactcttgacgtcagatgatccacgtgcctcggcctcccaaagtgctgggattacaggcgagagccaccgtgcccggcgcagaattcttttttttagagatgaggtattgccatcttgcccagacttgtctcgaactcctgggctcaaacaatccacccacctcggcctcccaaagtgctgagattactgacataagccaccatgcctggcccccagaattatgaatcctgtgaggatggcttcaaggtgagcgctgagccagacaaaaggatggggtttgggagcaccctgcttagactggaaagataatgttggagaagacttcctggaagaggggctttttgcgtagagttttgaagaatgagtaggagttctccagaggaggatgagtaactgcaataacacccagtttatcaagtgcctcctatgtgtctggccctgtgctttacccctcatttgaccacctctccagtgagagtctcagtcctttttttcctggtgaggaaacaggcatggcagagaggcatgacacatcaaggttgcccttcctggctccatctagcccgttctcctctgcttcctttgtttttcaccatctttagcctttgaccccaaccaaaaagagaagagaggaaatcccatgggcatagacagccacctcttaaactcttgtctggaatttttcacatagtaacaatgtctttttttcctccaaaaagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaaggtaagtgaaatcttagagatcccctcccaccccccaagcagcccccatatctaatcagggattcctcatcttgaaaagcccagacctacctttgagcctcagttgccccatctgtgccctgggtaggaatatcctggatccccttgggtctgatggggtagccgatgcctgatttgcacccacaacgtgggaggttataacctgtccccaagttgcaaatggggaaactgaggtacagggatgccagggaccctcctgaatgtgcacagcacctgggaaacccggggacggggtcgggcgtgcggtgtgggcatggtccctgacctgggctggggccaggatccaagagaaacaatgggggtgtggtgcatggatagaagtttgtgatgggcagagccagctggagctggttctgaatctgatgaaatcgcttgtcaaggcactgctaaagtcacatgcaacgagcgacttgttaggggaggaggagagtattttccccatctctgtgccctccatggaggctccaggggtgcagggcactggatgaggtctgggctgtggtgggacccaggagggggcaggcagaggggcacactcttagcccgccctaaacccatctgtgccgtgtgactccggcaaacctctcacccttgctaagcctagcaactaggttaaggcaccctcaaaccccagtctccccagtctccctaacacttgtaccagcctgaggggctgtgattcctagaggctcagtagtctggaatgtgatgggtttggcactcttcatttctcgggattacacctgtaatcccagcactttaggaggccaaggtgggagcatcgctttaggccagaacattgcagcctgggcagtatagcgagaatccatctctaccaaaaatgtttaaaaaaaattagccagacatgatggtgtgtgcctgtagtctcagttattcaggaggctgaggcgggaggagcacttgaatccaggagattgaggctgcactgagccatgagtgccccactgcactccagcctgggtaacagcaagaccataataaataaataaataagtgaagcaagtaagtttttgactgccagaagaaaagcagagccatggttggaatctcaggctgcctgaatatcagatatacattaattccttcaagtatttactaagcacctattgtgtgccaggcacagtgtcaaccaagctaagattctaagtagggaatatgaatgatacacaaggaaataaaatagggtgcttcctattttaggcaggctggaaggaagaggaggagccagccatactgagattcaggagattcaaggggagaaatgcccttttttttttttttttttttttttttgagagggagtcttcctctgttgcccaggctggagtgcagtggcacgatcttggctcactgcaacctccaccgccccgattcaagccattcttctgcctcagcctcccgagtagctgggatcacaggcgccagccaccatgcccagctaatttttggtatttttagtagaagcgggcgtttcaccatattggccacatatcggccaggctggtcttgaactcctgacctcaagtgatccacctgcatcggcctcccaaagtgctgggattacaggcgtgagccaccgcgcccgcacggagaaatgctcttggtaggggaaataacttgtgcaaaggcgccgccgccgtggtgagatccagcttggcgtgttaggggaaatgggcaccctcgttcctggaaaacggtaggcctgtgggtgaccagcttccctatctgtcttcccacagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttccctgtggtgtccaggataagggaaggtgaccttgagaagatcctcctctccctgacctcagtttctctaggagacctgggagggggtaagtggaaaggctcaaggaatgagtggcaaggtgttctcggagatgccaagccattggaaggtgcttacgacacctgtgggctctaagggcagccttggccattcctgactccctgactctctgtctctatttctctatctccttattgctgtttcttttttctttttattttttttagacggggtctcactctgttgcccaggccgaagtgcagtggcacaatcacagctcactgcagcctcaaccacccgggctcaatcgatcctcccacctcagccacctgagtagctgggactacaagtgcatgccatcatgcccaactaacttttgtattttttgtagaggctgagtttcgccatgttgcccaggctggtgtttctgttttggaaacagctgagaccccatacctttttaagtctcagcttccctccatctttcctcattcctcaaaacttctctctttggggtctgaggcagaggatctgtctctgtctttgtctctatcaccatcctgttgctctccccacttgggagtcacctttccaaacacaggcagctcagagccaagtctatctgcaaccaagtgccttgttttgtggtactgtccccaacctgaggggttagtcccatcacccctcctgtttgtggcacttccatgttagtgacacctcattctttcacatataaagagtagggagaaacaaataagttgtgaagaatattttcatgcacaccacatctcccaggttatatatacttcctgtttaggatgttggaattttgaggtgtgctgagtccagccccctctccacaccaaactgcctgcccatcctgaatctctgagaggaatggcgatgggtgtcttgagggacagagtacccacttccagcagtgaggtgtatctgctggga

SEQ ID NO: 5 - DNA, 2945 bp, synthetic construct
  source 1..2945: mol_type = other DNA
aaatcctggggaattggccacctcctcttctcctcttaggcatgaagcgcgtctggcttctccaaagaactcttcccctccactacctcagagttagcttcctctcttcagccagtgatcctggggtcccagacacaataattaaccaagagagggtgaaaggctccctgctgtgtttatgcaatggctcaggcccttgtgaagtgccgagggaccccaagcagcctccatctcccagggcatggtccatccccagcttcacagaacaggaaagctgtggaggagtgtgggcagcagggtaggaatggatatagcccttggcaacaacacatttccccacaaagcacccacccaaaagaacaacaacgatagttttagtttttagtaatgagaacaatagttctcatgactaaaagccatcagccaggacacgtgttctcaacccttttgcggtctttggaccctttgaaactctgacagaagccatggaggaatgttctcactgagtgcatgcactcaaaatgatgcattcaacttcaattcagtttcagggatgtatggcctgaccaccaatgcaggggattagcaatcgcaatagtggagagggcatgggagtgggaatctggctggatcaagcaagtggatgccagcagcccagaaaaagagcccccctacctgctttttccttctgggcactattgcccagcaaatgccttcctctttccgcttctcctacctccccacccaaaattttcattctgcacagtgattgccacattcacctggttgagaaaccagagactgtagcaactctggcagggagaagctgtctctgatggcctgaagctgtgggcagctggccaagcctaaccgctataaaaaggagctgcctctcagccctgcatgtctcttgtcagctgtctttcagaagacctggtaagtgggactgtctgggttggccccgcactttgggcttctcttggggagggtcagggaagtggagcagccttcctgagagaggagagagaaagctcagggaggtctggagcaaagatactcctggaggtggggagtgaggcagggataaggaaggagagtatcctccagcaccttccagtgggtaagggcacattgtctcctaggctggacttttcttgagcagagggtggggtggtaaggaaagtctacgggccccgtgtgtgtgcacatgtctctgtgtgaatggacccttccccttcccacacgtgtatccctatcatcccacccttcccaccagagccatagccatctgctggtttggttatttggagagtgcaggccaggacaaggccatcgcttggggcatgaatcctctgcgtactgccctggccagatgcaaattccctgccatgggattccccagaaggttctgtttttcaggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcagggtgaggaggggctgggtgtggcgggggctctctgcctggtcctggggctgccctgggccagcggtcctccctgccacccttcatagatggctatgcctcggctctctctgagatctttaaactctggcttcttcctcctcaatcttgacagaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcatgcctctctcttatgcttttgtggaatgaggttcctcggtgtggagggagggttggaaaacccaaaggaagaaaaagaaatctatgttatcccaccctacctctcacaagcctttcctgctttacccctcacctggcctctgccccacattcccttccagcccctccatcttgagccattggatttgaggcttaaggattccaaaaagtccatgacactatagctgatgattttactagtggttctgaaatgggtcggggatttgggaacagggtggtagtataagaacaactgatactgttctctaagctaaattttagcttccagctaaatgtcttagatgtggctcttgggaaattagagtgatagctacatagaagtgtgtgggtgtgtgtgtgtgtgtctgtgtgtgtgtgtgtgagagagagacagacagaaagagagcaagagagggaaggggggagaggctgattgtgtgtgtggtgtgatgtaggtggacaatgttcagagtcctccattaacaggataatcctcacacctgtccacatacctgtagtttgtccttggggattttgaaaatttttcctccctctccactcccaaactcccaactcaattaaatgataaaggaataggcaaataggaaaataaattagtaaaacttaagtcaaagaataggttattcatacgctgccctatgggattctatgctttggtgatcagaaaattatctaaaaaatacttcccaagggctggtacaaggggaaggccagaagacgagtggttcttctctgaggtggacattaaaaaaagaagaaaatgaaggggaaccttttgacaagaatgtcaccccaaactggattttcatgctgtggtgtggggaattttctgttgtcctcacttaggtgctggggcagtggtgttagtgatgggtaaaaaggtaggaagctgtcaccagaatcactaaaccagggttcttaacttgtctgtctatacatct

SEQ ID NO: 6 - DNA, 5170 bp, synthetic construct
  source 1..5170: mol_type = other DNA
ctgtggagtaggggaagggcactcctggggtggcaaggtgggaggtgggccctgtgttcccacagtgggcagggaggtagtgaaagggaagctggccggacaggcagggccattccaagagggctttgtgcgcagggctaagccaagctttctccataggcaatggggagcaactggaggttcgtagcaggagaaggacacatcttgcccaccaggaggctaagtaaaaacagttgtctcccaagttataagttcctggaacccttgctgggagcaggatttagaaaaatgatgctgagagatgctagaaacatattcgccctgaggctctctcactcagactgcaagaggaaggtatcatcagaattgcccttaaccaggaaccagaatagctgggtccccttcctgccaagtcagcaaccagctatgtgaccttgctcaggtccatctccgggtgtcagtttcttcatctacaatgcaagagggttgcccacctctgagaacccttctaaccccaaatctcaccctatgaatctaagaacaccaacccctcgccatcctaagtatcacagagccaggcaagcatgggtgagagctcagaccatccttgttggactaaaaggaaggggcagactgcccatggggggcagccgagagggtcaggcccccataggtcctcagcctgcttcaacctcaaaggggatggggggctgagtggtgccagaggagcagcaggctcgctcggggagagtagggccttaggatagaagggaaatgaactaaacaaccagcttcctcccaaaccagtttcaggccagggctgggaatttcacaaaaaagcagaaggcgctctgtgaacatttcctgccccgccccagcccccttcctggcagcattaccacactgctcacctgtgaagcaatcttccggagacagggccaaagggccaagtgccccagtcaggagctgcctataaatgccgagcctgcacagctctggcaaacactctgtgtggctcctcggctttggtaagtgagctgccagcttccccaggcagaagcctgcctgccgattccttctttccttccctgacccaacttccttccaaatcctcctcctagaagccctccttggttggccctgcctactttaaagcttctttcacattttcttaggtcatgttcccctggggcctcctgccctcaaatgctttgctttttggcactctgtagatattctaaaaaatcattttgtacatgtgtgtgacaggccatctcccagttaagttgcagcctgtgctttctttttattttgcacttcccccactatttctgtgagtgcttagtaggaagtgtcaaagaagcttgacagcattttcttctaagtgtcccaactcttggttttccattacacagacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaaggtagggctggactctggcaggtctgacccagcctcaccgcagtttgggttgacaagggaggatgggagtatgggctacagcaatcaaggggaagatttgagctcctggagcccagccccaagacgcagcgagtgtcctgttatacagggcaggtgctcacagttacacaggacgacagggtcaagaaattgctcaattgaacacctgctatttgtcgggccctgttctgggcagagggatgtagtggtaaatggggagcccactattccagtggagggagacacacagtaaagttgttggccaataaagagcacagataaagccaaatgccaataagtgcctggaagaaaatgagatagagtgcgctgtgggcaatggggctgggtggggtggaggtgaccagttagggtacatgagaagggcctctttgaggaggtaacatttgagctgagccccgaatgttggggagggaagcccctgaggatgacacttggcacaaagctgaggagaccctaagcctcaggcgggaacttggggtggaagacttgggggcttttctaatcctaagggtctgcggtggaaaatgaatgcataaagagcacatggagagcacctgcacagcactcagggaactgggaggtttttcccccgctccaaaaatgattaggcagttctaagaaaaaggctgagcacttccaacagcctttttgttttcttttcaaatttggggaaagtcgggaaacagaggcctgcattaagaagggtggaacacatgggtctcagtctcagttccagtcccggagccagacatcctggggtaggtccccagccctcccagtgcccctccctccgccttggtaaggtggagaattgcagccttcagagttaggggccctgacagctctccataggtggaggcctcaggcaggcaggatgctgggtggggtaggcaagaaagggcccagcagagaggccgcatggcaaaactatcctccatgtgaccccctatgcccgcttcaccccccacctgacatcccccaccagaagcaaagcgatgctgtgggaaaggaagcagagcctcatggatgggctgcacaggagagtgctcgcattggctgggtaccccacaggttctgggaggggacttagcgaggtgactcagccagagggtggcagagctggaaccaaccagtgctctcttggaccccgcctgggtctgggtcttccacctcccctgatgtctctcctttttttttttttttttttttttttgagacagagtttcgctcttattgcccaggctggagtgcaacggcacaatctcggctcaccacaacctccacctcctgggttcaagcgattctcctgcttcagcctccagaatagctgggattacaggcatgcaccaccatgcctggctaattttgttattttttgtagagacaaggtttctccatgttggtcaggctggtctcgaactcttgaccttaggtgatctgcctgcctcggcctcccaaagtgctgggattacaagcatgagccaccctgtccgaccatctccccttttatactttatcacacccttgaggtcagcggagcacatactctgctctctgaccctccatctcccctgcccacacctagtttttctaggtgtttccccgttgtattggttgaaataagtttcactaattggtaacctccagagggaagggaagggagggcaggggaaggagtgaagtgcagaggggtagcagagtggaactggcctctaagtcagatctgaatttgcatgccctcaatagtcaagctgtgaaaactaatgaccctctctaggactggtttcaagtcttcctccaggaagataccattcctagctgttaaagttgttataaggaccaaatgaggtgacatttccaggcttactcatgccatgaccagggcaagaccctggaactcagcttcctcttctataaatagagaatcagcacccaagtcacagggtcatggagggaataaactggagagcgtttggtatgtgctcagtgtctgctccattgtgcgcactcagcctatggtcatttttaatttttaaatccagccccagggtgaggcttcccttgtacatttgccagctggtcatttactgtgctcccagtccccacctctggccacacccagctctcacagccttctctccccacccgcagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccactaatcaggaggccaggccaccctgcctctacccaaccagggccccggggcctgttatgtcaaactgtcttggctgtggggctaggggctggggccaaataaagtctcttcctccaagtcagtgctctgtgtgcttcttccacctcttctccaaccctgccttcccagggctctgggcattagacagccctgtccttatctgtgactcagccccctcattcagtattaacaaaatgagaagcagcaaaacatgggtctgtgctgggccccttgggctcacctccctgaccatgtcctcacctctgacttcaggccccactgttcagatcccaggctccctgccccatctcagacaccctgtccagcctgtccagcctgacaaatggcccttgtcactgtacactgtagaaagcaaaaaggcatatctctaccccttgatatgcctgctacctcaccaaccagccccaagcctgtcttcacccatcactgtctacacagccctctctctctcctaacagaattctattcctctgaaagtcttccagaaatggacctagatagtgccatgtctggggaggaatactggcacccaggcagtggaaacaaggacagatccggtgtgtttactctcacactttttctggatccaagagagcaattggatctctcttaacagacctgccaccctaatcaacgggagtgctcacacaagtgggagtctgagagcttagccctatgcccaccctggtctcaacagaatggcagagtgaagaaatgagctccagtccgggctctgcagctaaaagactccgtgactaccaggaggagtctgtgctctgggtccagagagcctgggggtctgaagattctgattttaagctttgagggccagctgtcctttctggtgggtagctgagagctgggcccagcacaagtatctgcttctctctgcaaagcctactaccaggcctggccctagctgcctcagggctactgctgcagctcccccttcaagctgccccagatccaagtgctcagttcagggactcctctgccatcttcctgccctttggctataaagtccctttcctaggttataaccagtggggtttggcct

SEQ ID NO: 7 - protein, 267 aa, synthetic construct
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 8 - protein, 745 aa, synthetic construct
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 9 - protein, 308 aa, synthetic construct
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 10 - protein, 93 aa, synthetic construct
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 11 - protein, 114 aa, synthetic construct
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP